(19) United States
(12) Patent Application Publication
(10) Pub. No.: US 2023/0141913 A2
(43) Pub. Date: May 11, 2023

(54) PHENOXY(HETERO)ARYL ETHERS OF ANTIPROLIFERACTIVE ACTIVITY

(71) Applicant: XENIOPRO GmbH, Kelkheim (Taunus) (DE)

(72) Inventors: Viktoria REINMÜLLER, Allschwil (CH); Roman MARTY, Allschwil (CH); Olivier WAGNIÉRES, Lausanne (CH); Jean-Baptiste GUALTIEROTTI, Allschwil (CH); Verena KÜPPERS, Binningen (CH)

(73) Assignee: XENIOPRO GmbH, Kelkheim (Taunus) (DE)

(21) Appl. No.: 17/270,652

(22) PCT Filed: Aug. 23, 2019
(86) PCT No.: PCT/EP2019/072642

§ 371 Date: Feb. 23, 2021

(30) Foreign Application Priority Data
Aug. 24, 2018 (EP) 18190774.2

(51) Int. Cl. A61K 31/5375; C07C 43/275; C07D 213/647; A61K 31/09; A61K 31/44; A61P 35/02; A61P 35/00; C07D 295/096; C07D 453/02; A61K 31/351; A61K 31/451; A61K 31/495; A61K 31/439; C07D 305/06; C07D 331/04; C07D 205/04; A61K 31/337; A61K 31/38; A61K 31/397; C07D 407/12; C07D 411/12; C07D 405/12; C07D 409/12; C07D 401/12; A61K 31/5377; A61K 31/496; C07D 309/04; A61K 31/12; A61K 31/085; C07C 49/255; C07C 43/295; C07C 205/04; C07C 251/20; C07C 251/48; A61K 31/04; A61K 31/15; A61K 39/39

(52) U.S. Cl. CPC A61K 31/5375; C07C 43/275; C07D 213/647; A61K 31/09; A61K 31/44; A61P 35/02; A61P 35/00; C07D 295/096; C07D 453/02; A61K 31/351; A61K 31/451; A61K 31/495; A61K 31/439; C07D 305/06; C07D 331/04; C07D 205/04; A61K 31/337; A61K 31/38; A61K 31/397; C07D 407/12; C07D 411/12; C07D 405/12; C07D 409/12; C07D 401/12; A61K 31/5377; A61K 31/496; C07D 309/04; A61K 31/12; A61K 31/085; C07C 49/255; C07C 43/295; C07C 205/04; C07C 251/20; C07C 251/48; A61K 31/04; A61K 31/15; A61K 39/39

(57) ABSTRACT

The present invention comprises novel aromatic molecules, which can be used in the treatment of pathological conditions, such as cancer, skin diseases, muscle disorders, and immune system-related disorders such as disorders of the haematopoietic system including the haematologic system in human and veterinary medicine.

### CROSS REFERENCE TO RELATED APPLICATION

This application is a 35 U.S.C. 371 National Phase Entry Application from PCT/EP2019/072642, filed Aug. 23, 2019, which claims the benefit of European Patent Application No. 18190774.2 filed on Aug. 24, 2018, the disclosure of which is incorporated herein in its entirety by reference.

The present invention relates to novel compounds and their use as therapeutic agents in human and veterinary medicine. The compounds of the present invention can be used in the treatment of pathological conditions including cancer, skin disorders, muscle disorders, disorders of the lung, disorders of the haematopoietic system including the haematologic system and immune system-related disorders.

### DESCRIPTION OF THE INVENTION

The present invention covers novel molecules that show remarkable biological activity on human and animal derived cells. According compounds were found to influence the growth and survival of cancer cells and primary non-cancer cells. In particular, molecules were identified that are able to completely or partially inhibit cell growth or result in cell death.

Thus, the present invention relates to compounds as defined herein that feature antiproliferative activity, which can be used in the treatment of benign and malignant hyperproliferative disorders in human and veterinary medicine. In particular, the present invention relates to compounds as defined herein for the treatment of disorders of the haematopoietic system including the haematologic system and immune system-related disorders, concerning malignancies of both the myeloid lineage and the lymphoid lineage, malignant and non-malignant disorders of the skin and mucosa, e.g. cornification disorders, malignant and non-malignant disorders of the muscle, including hyperproliferative disorders of the muscle, such as muscle hyperplasia and muscle hypertrophy, disorders of the neuroendocrine system, hyperproliferative disorders, cancer and pre-cancerous lesions of the skin and mucosa, such as non-melanoma skin cancer including squamous and basal cell carcinoma, actinic keratosis, hyperproliferative disorders and cancer of the oral cavity and tongue, hyperproliferative disorders and cancer of the neuroendocrine system such as medullary thyroid cancer, hyperproliferative disorders and cancer of the haematopoietic system including the haematologic system such as leukemia and lymphoma, hyperproliferative disorders and cancer of the lung, breast, stomach, genitourinary tract, e.g. cervical cancer and including cancer of the ovaries, in human and veterinary medicine.

The compounds of the present invention relate to bisarylether structures composed of two six-membered aromatic cycles, wherein one of the aromatic cycles is an unsubstituted or substituted benzyl ring and the other aromatic cycle is an unsubstituted or substituted aryl ring, which optionally contains N-atoms, thus optionally being a six-membered heteroaromatic cycle. All such bisarylether structures share the common feature of containing a substituent in both para-positions relative to the ether bond, wherein such substituent on the benzyl ring which cannot be a heteroaromatic cycle, is preferably selected from apolar residues and/or from sterically demanding residues; and wherein such substituent on the aryl ring which can optionally be a heteroaromatic cycle, is selected from structural units preferably containing a high amount of heteroatoms.

A first aspect of the present invention relates to compounds of general formula (I) and salts and solvates thereof:

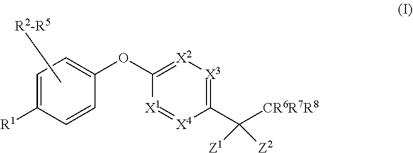

R¹=C₁-C₁₂ preferably C₄-C₁₂ alkyl, C₂-C₁₂ preferably C₄-C₁₂ alkenyl, C₂-C₁₂ preferably C₄-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, —OC₁-C₁₂ preferably —OC₃-C₁₂ alkyl, —OC₂-C₁₂ preferably —OC₃-C₁₂ alkenyl, —OC₂-C₁₂ preferably —OC₃-C₁₂ alkynyl, —OC₃-C₈ cycloalkyl, —OC₅-C₈ cycloalkenyl, —OC₅-C₁₂ bicycloalkyl, —OC₇-C₁₂ bicycloalkenyl, —OC₈-C₁₄ tricycloalkyl, —SC₁-C₁₂ preferably —SC₃-C₁₂ alkyl, —SC₂-C₁₂ preferably —SC₃-C₁₂ alkenyl, —SC₂-C₁₂ preferably —SC₃-C₁₂ alkynyl, —SC₃-C₈ cycloalkyl, —SC₅-C₈ cycloalkenyl, —SC₅-C₁₂ bicycloalkyl, —SC₇-C₁₂ bicycloalkenyl, —SC₈-C₁₄ tricycloalkyl, —NHR⁹ or —NR⁹R¹⁰ wherein R⁹ and R¹⁰ are independently from each other selected from: C₁-C₁₂ preferably C₃-C₁₂ alkyl, C₂-C₁₂ preferably C₃-C₁₂ alkenyl, C₂-C₁₂ preferably C₃-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, or wherein R⁹ can form a ring structure together with R¹⁰ wherein the said ring structure including the N-atom is selected from three to eight membered cyclic structures or five to twelve membered bicyclic structures and wherein all said ring structures can additionally contain one or more heteroatoms independently selected from O, S and N in replacement of a carbon atom contained in the ring structure, and particularly wherein such a replacement results in residues that contain at least twice the number of C atoms than heteroatoms independently selected from O, S and N;

wherein all alkyl, alkenyl and alkynyl residues contained in the definitions of R₁, R⁹ and R¹⁰ are linear or branched, and are unsubstituted or substituted with one or more substituents independently selected from: —F, —Cl, —Br, —I, —CN, —NCO, —NCS, —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₁-C₅ alkyl such as —OCH₃, —OC₃-C₅ cycloalkyl such as —O(cyclopropyl), linear or branched —NH(C₁-C₅ alkyl), linear or branched —N(C₁-C₅ alkyl)(C₁-C₅ alkyl), —NH(C₃-C₈ cycloalkyl) such as —NH(cyclopropyl), —N(C₃-C₈ cycloalkyl)(C₃-C₈ cycloalkyl), linear or branched —N(C₁-C₅ alkyl)(C₃-C₈ cycloalkyl);

wherein when an alkyl, alkenyl and alkynyl residue contained in the definitions of R¹, R⁹ and R¹⁰ is substituted with one or more substituents being ═O, such substitution with ═O cannot result in one of the groups selected from C═O, S═O and N═O directly bound to an aromatic ring;

wherein all cyclic structures, bicyclic structures and tricyclic structures including cycloalkyl, cycloalkenyl, bicycloalkyl, bicycloalkenyl and tricycloalkyl residues contained in the definitions of R¹, R⁹ and R¹⁰ are unsubstituted or substituted with one or more substituents independently selected from: —F, —Cl, —Br, —I, —CN, —NCO, —NCS, —OH, —NH₂, —NO₂, ═O, linear or branched C₁-C₅ alkyl such as —CH₃, linear or branched —OC₁-C₅ alkyl such as —OCH₃, linear or branched —NH(C₁-C₅ alkyl), linear or branched —N(C₁-C₅ alkyl)(C₁-C₅ alkyl), —NH(C₃-C₈ cycloalkyl) such as —NH(cyclopropyl), —N(C₃-C₈ cycloalkyl)(C₃-C₈ cycloalkyl), linear or branched —N(C₁-C₅ alkyl)(C₃-C₅ cycloalkyl);

wherein all alkyl, alkenyl and alkynyl residues contained in the definitions of R¹, R⁹ and R¹⁰ can contain one or more heteroatoms independently selected from O, S and N in replacement of a carbon atom, and wherein such a replacement results in residues that contain at least twice the number of C atoms than heteroatoms independently selected from O, S and N, and wherein such replacement additionally cannot result in one of the groups selected from C═O, S═O and N═O directly bound to an aromatic ring;

wherein all cycloalkyl, cycloalkenyl, bicycloalkyl, bicycloalkenyl and tricycloalkyl residues contained in the definitions of R¹, R⁹ and R¹⁰ can contain one or more heteroatoms independently selected from O, S and N in replacement of a carbon atom, and wherein such a replacement results in residues that contain at least the same number of C atoms than heteroatoms independently selected from O, S and N;

wherein all alkyl, alkenyl, alkynyl, cycloalkyl, cycloalkenyl, bicycloalkyl, bicycloalkenyl and tricycloalkyl residues contained in the definitions of R¹, R⁹ and R¹⁰ can be partially or fully halogenated, particularly fluorinated, more particularly perfluorinated;

wherein bicyclic and tricyclic residues include fused, bridged and spiro systems; and wherein R¹ is preferably selected from methyl, ethyl, n-propyl, n-butyl, n-pentyl, n-hexyl, iso-propyl, sec-butyl, tert-butyl, tert-pentyl, tert-octyl, 3-pentyl, —CF₃, —CF₂CF₃, —(CF₂)₂CF₃, —CH(CF₃)₂, —CH₂SCH₃, —CH₂CH₂SCH₃, —CH₂SCH₂CH₃, —CH₂CH₂SCH₂CH₃, methoxymethyl, methoxyethyl, methoxypropyl, ethoxymethyl, ethoxyethyl, propoxymethyl, dimethyl-aminomethyl, dimethyl-aminoethyl, diethyl-aminomethyl, ethyl-methyl-aminomethyl, cyclopropyl, methyl-cyclopropyl, ethyl-cyclopropyl, trifluoromethyl-cyclopropyl, perfluoroethyl-cyclopropyl, cyclobutyl, cyclopentyl, cyclohexyl, cycloheptyl, bicyclopentyl, bicyclohexyl, bicycloheptyl preferably norbornyl, bicyclooctyl, bicyclooctenyl, bicyclononyl, methylbicyclononyl, adamantyl, tricyclodecyl, oxiranyl, oxetanyl, tetrahydrofuranyl, methyltetrahydrofuranyl, trimethyltetrahydrofuranyl, tetrahydropyranyl, aziridinyl, N-methylaziridinyl, azetidinyl, N-methylazetidinyl, difluoroazetidinyl, pyrrolidinyl, N-methylpyrrolidinyl, piperidinyl, N-methylpiperidinyl, difluoropiperidinyl, thiiranyl, thietanyl, tetrahydrothiophenyl, tetrahydrothiopyranyl, dioxanyl, piperazinyl, dimethylpiperazinyl, dithianly, morpholinyl, N-methylmorpholinyl, thiomorpholinyl, N-methylthiomorpholinyl, oxa-azaspiroheptyl, N-methyloxa-azaspiroheptyl, azaspiroheptyl, N-methylazaspiroheptyl, thia-azaspiroheptyl, N-methylthia-azaspiroheptyl, difluorothia-azaspiroheptyl, azaspirooctyl, N-methylazaspirooctyl, oxa-azaspirooctyl, N-methyloxa-azaspirooctyl, oxa-azaspirononyl, N-methyloxa-azaspirononyl, azaspirononyl, N-methylazaspirononyl, oxa-azaspirodecyl, N-methyloxa-azaspirodecyl, azaspirodecyl, N-methylazaspirodecyl, dihydro-oxazinyl, N-methyldihydro-oxazinyl, oxazolidinyl, N-methyloxazolidinyl, dioxolanyl, imidazolidinyl, N-methylimidazolidinyl, N,N-dimethylimidazolidinyl, azepanyl, N-methylazepanyl, azaspirohexyl, N-methylazaspirohexyl, oxa-azadispirodecyl, N-methyloxa-azadispirodecyl, azadispirodecyl, N-methylazadispirodecyl, oxa-azabicyclooctyl, N-methyloxa-azabicyclooctyl, azabicyclooctyl, N-methylazabicyclooctyl, azabicycloheptyl, N-methylazabicycloheptyl, azabicyclononyl, N-methylazabicyclononyl, azaadamantyl, —O(adamantyl), oxa-azabicyclononyl, N-methyloxa-azabicyclononyl, oxa-azabicycloheptyl, N-methyloxa-azabicycloheptyl, diazabicyclooctyl, N-methyldiazabicyclooctyl, N,N-dimethyldiazabicyclooctyl, diazabicycloheptyl, N-methyldiazabicycloheptyl, N,N-dimethyldiazabicycloheptyl; 4-oxocyclohexyl; 3-oxocyclopentyl; 2-oxocyclobutyl, 4-oxobicyclo[4.1.0]heptan-1-yl;

and wherein R¹ is even more preferably selected from C₄-C₁₂ alkyl, C₄-C₁₂ alkenyl, C₄-C₁₂ alkynyl, cyclic, bicyclic and tricyclic residues, wherein the alkyl, alkenyl and alkynyl residues are preferably branched, including:

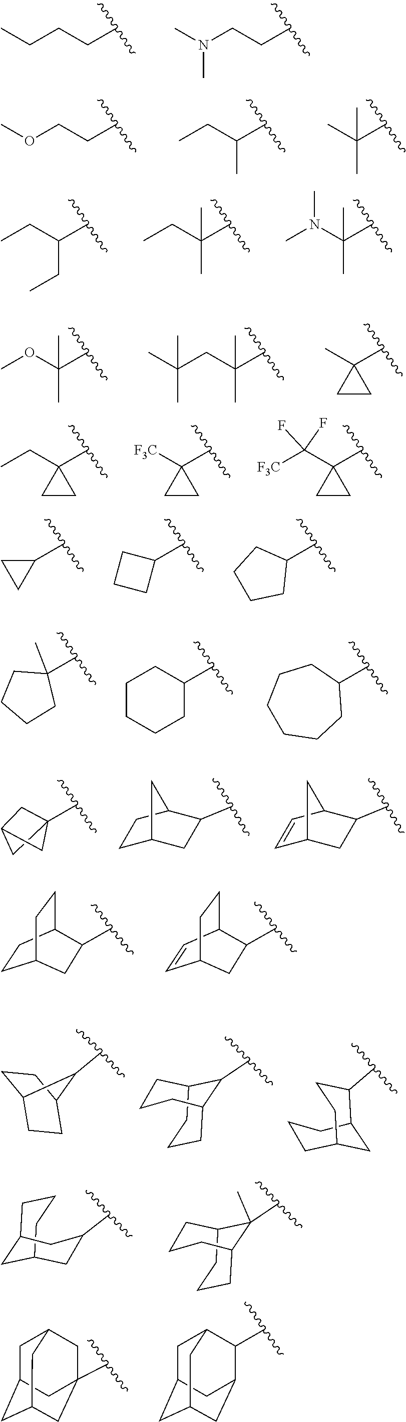

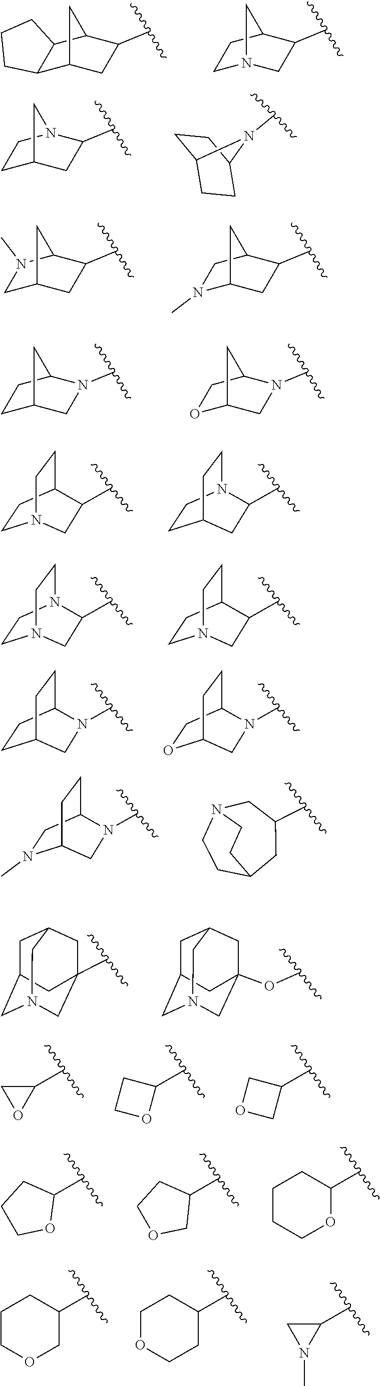

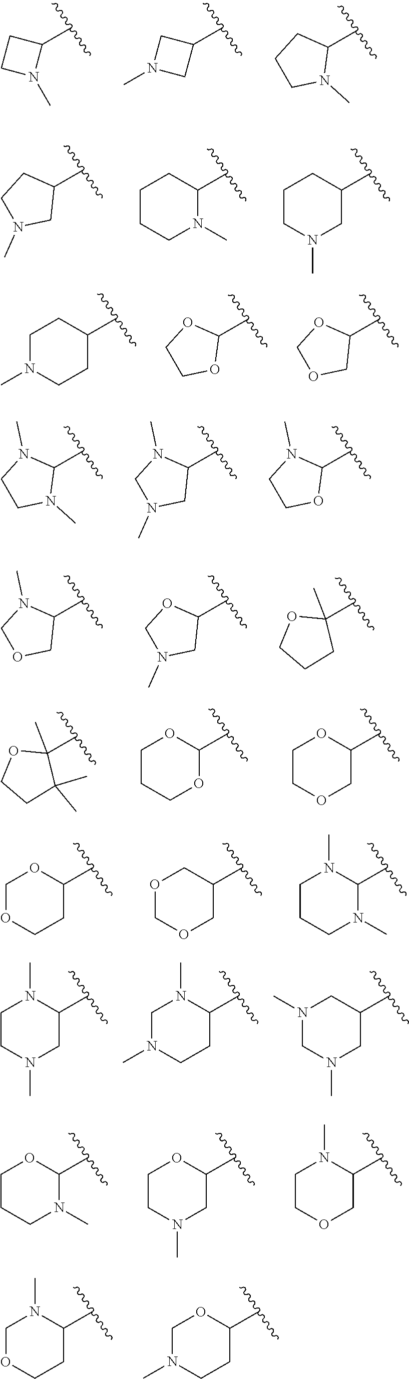

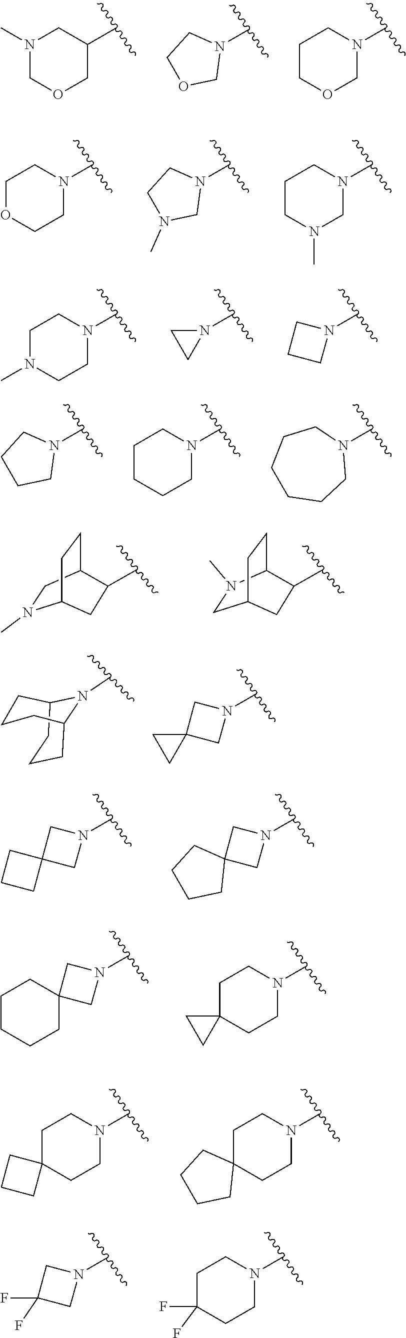

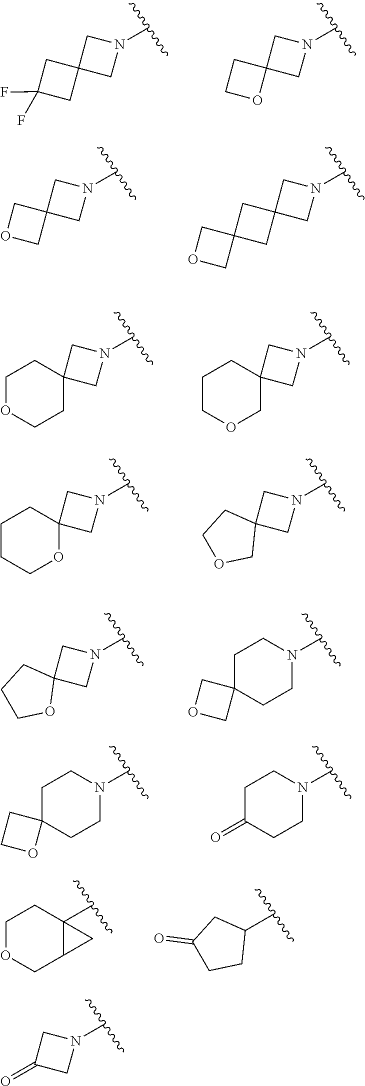

R²-R⁵ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl and cycloalkyl residues contained in the definitions of R²-R⁵ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂; wherein all alkyl, alkenyl, alkynyl and cycloalkyl residues contained in the definitions of R²-R⁵ can contain one or more heteroatoms independently selected from O, S and N in replacement of a carbon atom, and wherein such a replacement cannot result in one of the groups selected from C═O and S═O directly bound to an aromatic ring;

wherein R²-R³ each are preferably —H, R⁴ is preferably —H or —F, and R⁵ is preferably —H, —F, —Cl, —Br, —CH₃, —CF₃, —CH═CH₂, —C—CH, —CH₂OH, —CH₂NHCH₃, —OH, —OCH₃, —OCF₃, cyclopropyl, oxiranyl, —CH₂—N-morpholinyl, —C(CH₃)₃, —CH₂OCH₃, —NO₂, —CN, —NH₂, —N(CH₃)₂, —OCH(CH₃)₂, —CH₂NH₂, —CH₂N(CH₃)₂;

wherein the six-membered aromatic ring, to which substituents R¹ to R⁵ are bound as defined in general formula (I), is preferably selected from:

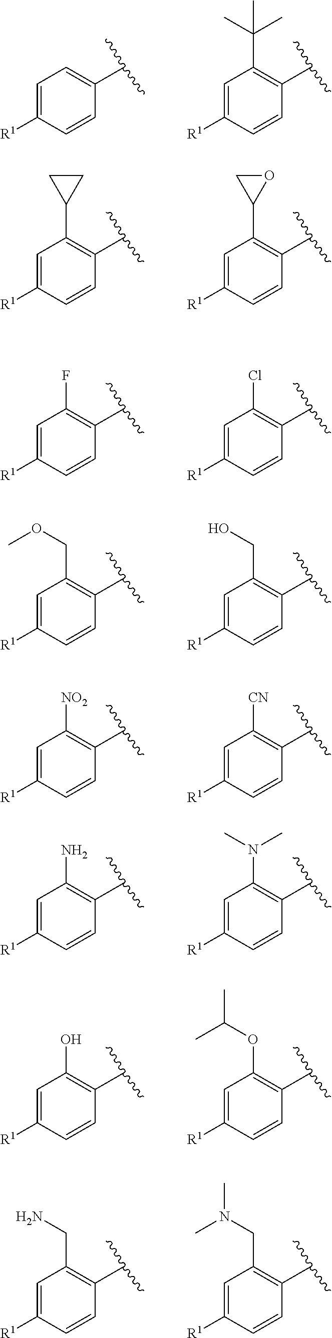

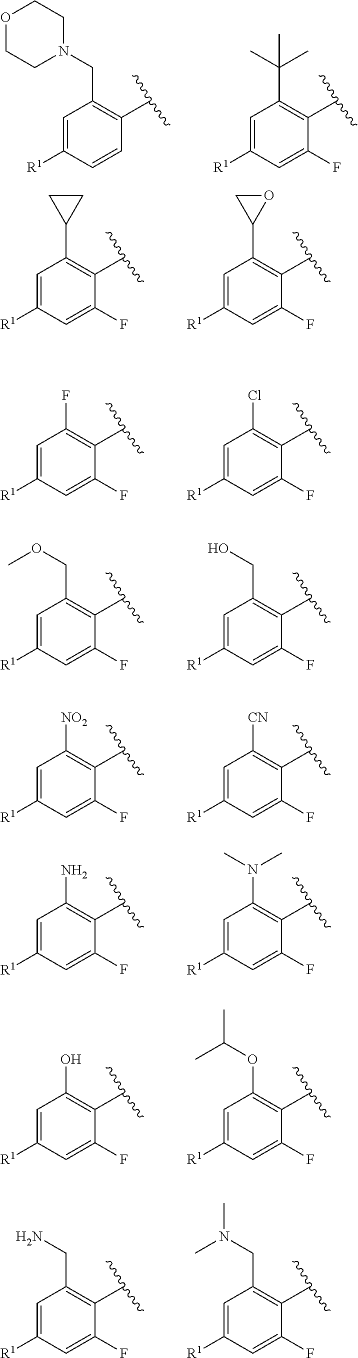

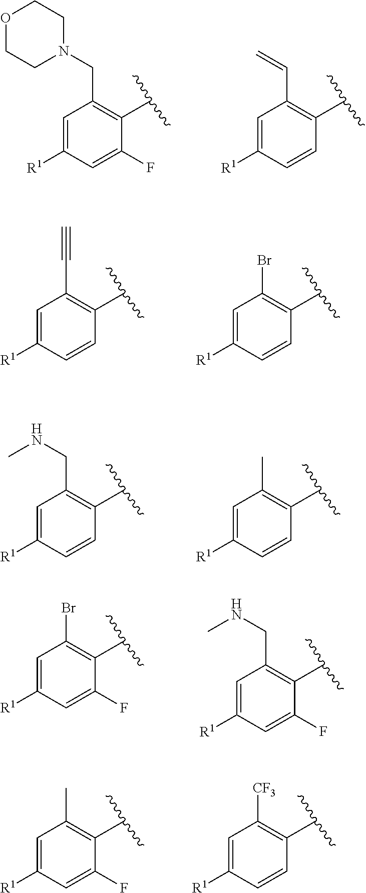

X¹-X⁴ are independently from each other selected from N, CR¹¹, CR¹², CR¹³, CR¹⁴;

R₁₁-R¹⁴ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl and cycloalkyl residues contained in the definitions of R¹¹-R¹⁴ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂; wherein all alkyl, alkenyl, alkynyl and cycloalkyl residues contained in the definitions of R¹¹-R¹⁴ can contain one or more heteroatoms independently selected from O, S and N in replacement of a carbon atom, and wherein such a replacement cannot result in one of the groups selected from C═O and S═O directly bound to an aromatic ring;

wherein R¹¹-R¹⁴ are preferably selected from —H, —F, —Cl, —Br, —CH₃, —CF₃, —OH, —OCH₃, —OCF₃, cyclopropyl, oxiranyl, —C(CH₃)₃, —N(CH₃)₂, —NH₂, —CN, —CH₂OCH₃, —OCH(CH₃)₂, —CH₂NH₂, —CH₂N(CH₃)₂, —CH₂OH, —NO₂, —CH₂—N-morpholinyl;

and wherein the six-membered aromatic ring containing X¹-X⁴ as defined in general formula (I) is preferably selected from:

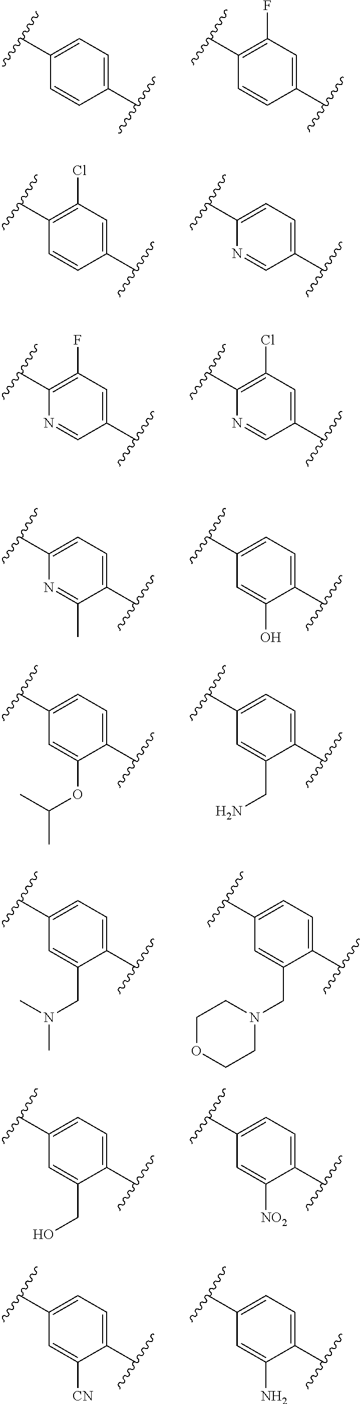

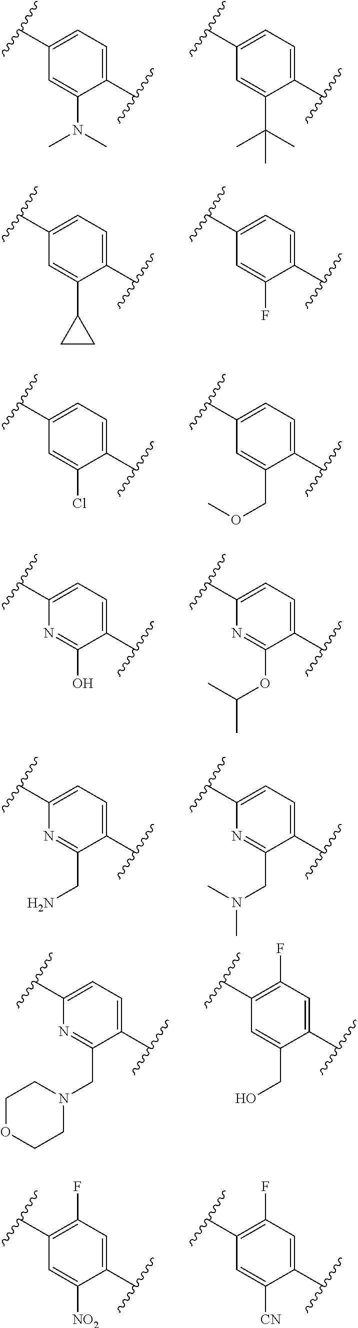

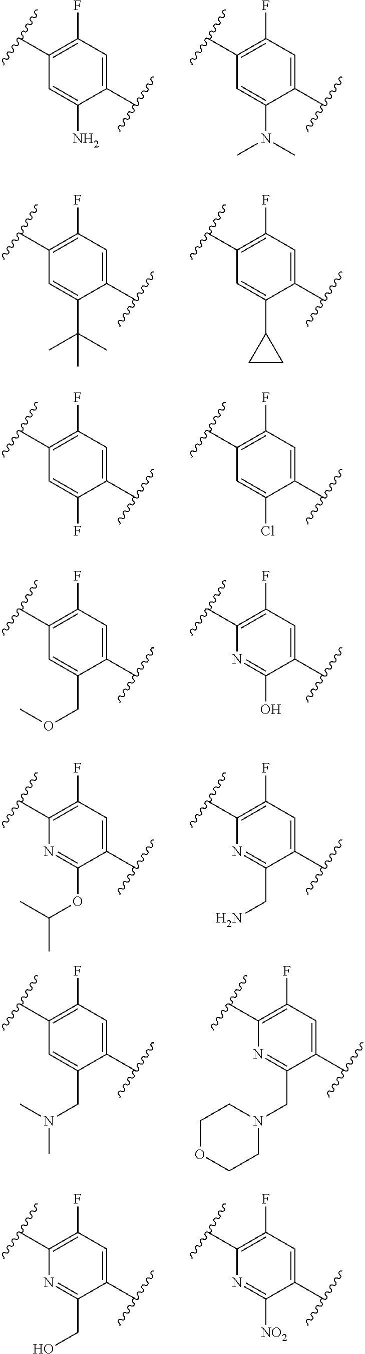

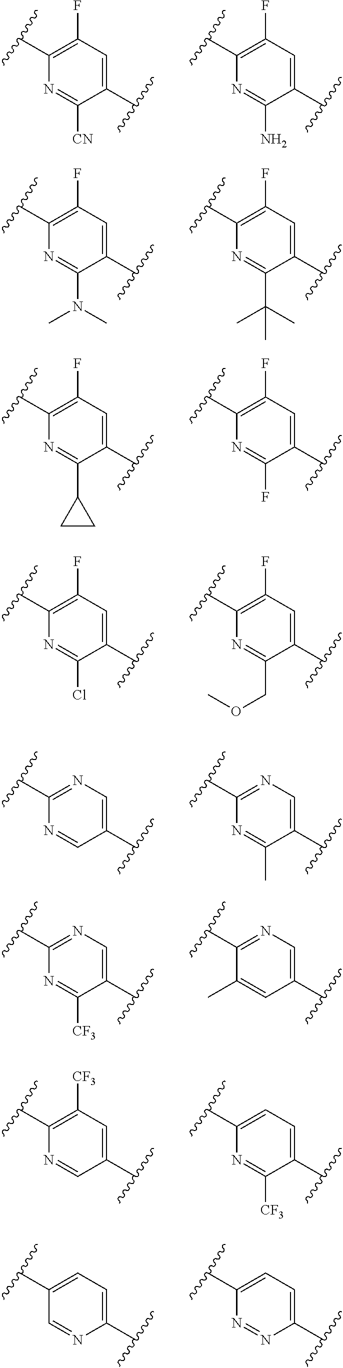

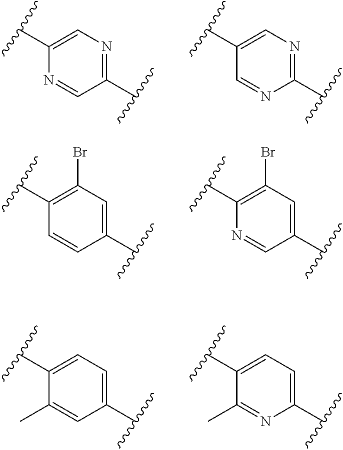

R⁶ and R⁷ are independently selected from —H, —F, —CH₃; or R⁶ and R⁷ form together a cyclic residue including the carbon atom to which they are bound and wherein the cyclic residue is C₃ cycloalkyl;

R⁸ is selected from —H, C₁-C₃ alkyl preferably —CH₃, C₂-C₃ alkenyl, C₂-C₃ alkynyl, —F, —CF₃ and aromatic and heteroaromatic residues preferably six-membered aromatic cycles and five to six membered heteroaromatic cycles;

wherein said aromatic and heteroaromatic residues contained in the definition of R⁸ can optionally be linked through a C₁ alkylene or a C₂ alkylene linker to the carbon atom to which R⁸ is bound;

wherein all aromatic and heteroaromatic residues contained in the definition of R⁸ are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃alkyl), —NH(cyclopropyl), —N(cyclopropyl)₂, linear or branched —N(C₁-C₃ alkyl)(cyclopropyl);

wherein all heteroaromatic residues contained in the definition of R⁸ can contain one or more heteroatoms independently selected from O, S and N in replacement of a carbon atom; wherein all alkyl, alkenyl, alkynyl residues contained in the definition of R⁸ are linear or branched, and are unsubstituted or substituted with one or more substituents independently selected from: —F, —Cl, —Br, —I, —CN, —NCO, —NCS, —OH and —NH₂; wherein R⁸ is preferably —H, —F, —CH₃, —CH₂CH₃—CF₃, —C₆H₅;

wherein all alkyl, alkenyl, alkynyl, cycloalkyl, cycloalkenyl, bicycloalkyl, bicycloalkenyl, tricycloalkyl, aromatic and heteroaromatic residues contained in the definitions of R²-R⁸ and R¹¹-R¹⁴ can be partially or fully halogenated, particularly fluorinated, more particularly perfluorinated;

Z¹ and Z² are selected from the following groups:

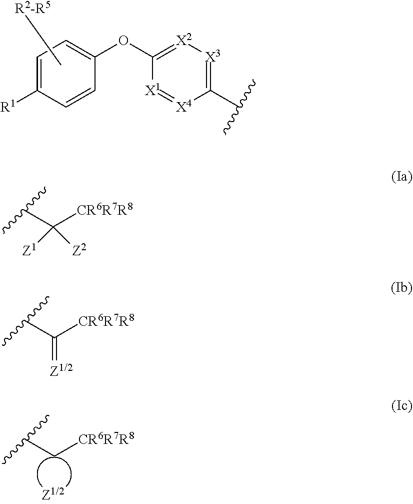

wherein Z¹ is selected from —H, linear or branched C₁-C₃ alkyl preferably —CH₃, cyclopropyl, oxiranyl, N-methyl-aziridinyl, thiiranyl, —N₃, —CF₃, —CF₂CF₃, and wherein Z² is independently selected from linear or branched C₁-C₃ alkyl preferably —CH₃, —CF₃, —CF₂CF₃, —OS(O)₂CH₃, —OS(O)₂CF₃, —OS(O)₂C₆H₄CH₃, —CN and —OR¹⁵ (general formula Ia), wherein R¹⁵ is selected from —H, C₁-C₈ preferably C₁-C₄ alkyl, C₂-C₈ preferably C₂-C₄ alkenyl, C₂-C₈ preferably C₂-C₄ alkynyl, C₃-C₆ cycloalkyl, C₅-C₆ cycloalkenyl, C₅-C₁₂ bicycloalkyl, C₇-C₁₂ bicycloalkenyl, C₈-C₁₄ tricycloalkyl, and aromatic and heteroaromatic residues preferably five- to six-membered aromatic cycles and five to six membered heteroaromatic cycles;

and wherein bicyclic and tricyclic residues include fused, bridged and spiro systems;

wherein said cycloalkyl, cycloalkenyl bicycloalkyl, bicycloalkenyl, tricycloalkyl, aromatic and heteroaromatic residues contained in the definition of R¹⁵ can optionally be linked through a C₁ alkylene or a C₂ alkylene or a C₃ alkylene linker to the O to which R¹⁵ is bound;

wherein all aromatic and heteroaromatic residues contained in the definition of R¹⁵ are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl, bicycloalkyl, bicycloalkenyl and tricycloalkyl residues, and alkylene linkers contained in the definition of R¹⁵ are linear or branched, and are unsubstituted or substituted with one or more substituents independently selected from: —F, —Cl, —Br, —I, —CN, —NCO, —NCS, —OH, —NH₂, ═O, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl, bicycloalkyl, bicycloalkenyl, tricycloalkyl and heteroaromatic residues, and alkylene linkers contained in the definition of R¹⁵ can contain one or more heteroatoms independently selected from O, S and N in replacement of a carbon atom;

wherein all alkyl, alkenyl, alkynyl, cycloalkyl, cycloalkenyl, bicycloalkyl, bicycloalkenyl, tricycloalkyl, and heteroaromatic residues, and alkylene linkers contained in the definition of R¹⁵ can be partially or fully halogenated, particularly fluorinated, more particularly perfluorinated

wherein R¹⁵ is preferably —H, —CH₃, —CH₂CH₃, n-propyl, isopropyl, cyclopropyl, benzyl;

wherein Z¹ is preferably —H, —CH₃, —CF₃ and cyclopropyl; and/or wherein Z² is preferably —OH, —OS(O)₂CH₃, —OS(O)₂CF₃, —OS(O)₂—C₆H₄-Me and —CN; e.g.:

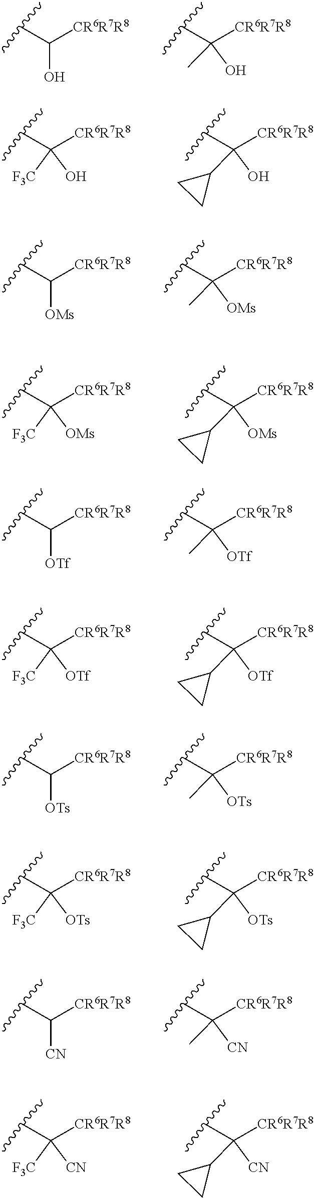

or wherein Z¹ and Z² are together ═O, ═S, ═NR¹⁶, or zwitterionic ═N^{[+]}R¹⁷O^{[-]} (general formula Ib); wherein R¹⁶ is selected from —H, —OH, —OCH₃, —CN, —S(O)CH₃, —S(O)CF₃, —S(O)C(CH₃)₃, —S(O)₂CH₃, —S(O)₂CF₃, linear or branched C₁-C₃ alkyl preferably —CH₃, cyclopropyl, —CF₃, —CF₂CF₃, —CH₂CF₃, —C₆H₅ and —CH₂C₆H₅; wherein R¹⁷ is selected from linear or branched C₁-C₃ alkyl, preferably —CH₃, cyclopropyl, —C₆H₅ and —CH₂C₆H₅;

wherein Z¹ and Z² are together preferably ═O, ═NR¹⁶ or zwitterionic ═N^{[+]}R¹⁷O^{[-]}; wherein R¹⁶ is preferably selected from —H, —OH, —OCH₃, —CH₃, cyclopropyl, and —CH₂C₆H₅; wherein R¹⁷ is preferably —CH₃, —C(CH₃)₃ and —CH₂C₆H₅:

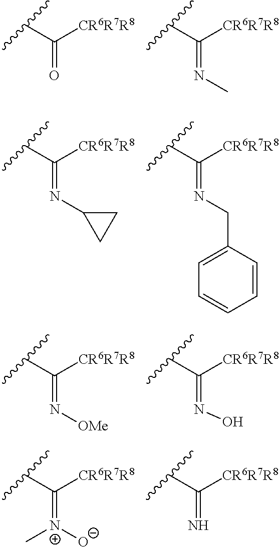

or wherein Z¹ and Z² form together a cyclic residue including the carbon atom to which they are bound (general formula Ic); wherein the cyclic residue is selected from three-membered rings, four-membered rings, five-membered rings and six-membered rings, wherein all rings optionally can contain one or more heteroatoms independently selected from O, S and N in replacement of a carbon atom; wherein all rings are unsubstituted or substituted with one or more substituents independently selected from: —F, —Cl, —Br, —I, —CN, —NCO, —NCS, —OH, —OCH₃, —NH₂, —NHCH₃, —N(CH₃)₂, ═O, —CH₃ and —CF₃;

wherein Z¹ and Z² form together preferably a three membered or four membered or five membered cyclic residue including the carbon atom to which they are bound; wherein this cyclic residue is preferably selected from cyclopropyl, cyclobutyl, oxiranyl, oxetanyl, aziridinyl, azetidinyl, thietanyl, thiazolidinyl, methylthiazolidinyl, thiazolidine-dionyl, methylthiazolidine-dionyl and oxazolidinyl, methyloxazolidinyl, oxazolidine-dionyl and methyloxazolidine-dionyl; and wherein this cyclic residue is optionally substituted preferably with —F, —OH, —OCH₃, —NH₂, —NHCH₃, —N(CH₃)₂, ═O, —CH₃ and —CF₃;

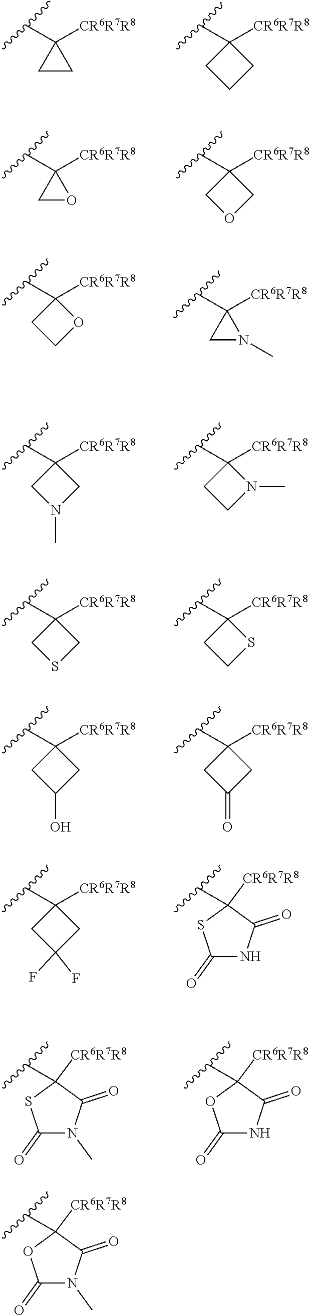

wherein all alkyl and cyclic residues contained in the definitions of Z¹ and Z² can be partially or fully halogenated, particularly fluorinated, more particularly perfluorinated.

Following preferred definitions of R¹-R¹⁷, X¹-X⁴, Z¹ and Z² may be optionally independently and/or in combination applied on all aspects including preferred and certain aspects, on all embodiments including preferred and certain embodiments, and on all subgenera as defined in the present invention:
1) R¹ preferably contains four or more preferably six or more and even more preferably seven or more carbon atoms;
2) R¹ is preferably selected from branched alkyl, alkenyl and alkynyl residues; 3) R¹ is preferably selected from cyclic, bicyclic and tricyclic structures, wherein bicyclic and tricyclic residues include fused, bridged and spiro systems;
4) R¹ preferably contains no heteroatom;
5) R¹ is preferably selected from cyclohexyl, norbornyl, bicyclooctyl, bicyclononyl, methylbicyclononyl, tricyclodecyl and most preferably adamantyl, e.g. 1-adamantyl and 2-adamantyl;
6) R¹ preferably contains one or more heteroatoms, preferably one, two or three heteroatoms independently selected from O, S and N in replacement of a carbon atom contained in R¹;
7) R¹ is preferably selected from tetrahydropyranyl, N-methylpiperidinyl, morpholinyl, 4-oxocyclohexyl, azabicycloheptyl, N-methylazabicycloheptyl, oxa-azabicycloheptyl, N-methyldiazabicycloheptyl, azabicyclooctyl, diazabicyclooctyl, N-methyldiazabicyclooctyl, oxa-azabicyclooctyl, azabicyclononyl, azaadamantyl and —O(adamantyl);
8) preferably two, or more preferably three of the substituents independently selected from R²—R⁵ are —H, i.e. preferably two and more preferably one of the substituents independently selected from R²-R⁵ are different from —H;
9) in the case that two of the substituents independently selected from R²-R⁵ are different from —H and are in ortho position relative to the ether bond, these two substituents are preferably different from —F, —Cl, —Br, —I and —NO₂ and more preferably different from each other;
10) the composition of ring atoms as defined by X¹-X⁴ is preferably selected from the cases that all of X¹-X⁴ are independently selected from CR¹¹, CR¹², CR¹³, CR¹⁴, or that one of X¹-X⁴ is N and the other three are independently selected from CR¹¹, CR¹², CR¹³, CR¹⁴, or that two of X¹-X⁴ are N and the other two are independently selected from CR¹¹, CR¹², CR¹³, CR¹⁴; i.e. the aromatic or hetoromatic ring is selected from benzene, pyridine, pyrimidine, pyridazine and pyrazine;
11) preferably two, or more preferably three of the substituents independently selected from R¹¹-R¹⁴ are —H, i.e. preferably two and more preferably one of the substituents independently selected from R¹¹-R¹⁴ are different from —H;
12) in the case that two of the substituents independently selected from R¹¹-R¹⁴ are different from —H and are in ortho position relative to the ether bond, these two substituents are preferably different from —F, —Cl, —Br, —I and —NO₂ and more preferably different from each other;
13) R⁶, R⁷ and R⁸ are preferably each —F;
14) R⁶ and R⁷ preferably form together a cyclic residue including the carbon atom to which they are bound and wherein the cyclic residue is cyclopropyl.

A preferred aspect of the present invention relates to compounds of general formula (I) and salts and solvates thereof, wherein R⁶, R⁷ and R⁸ are each —F,

and R¹-R⁵, R⁹-R¹⁷, X¹-X⁴, Z¹ and Z² are defined as in general formula (I) including the substitutions and preferred definitions.

A further preferred aspect of the present invention relates to compounds of general formula (Ia) and salts and solvates thereof, wherein R⁶, R⁷ and R⁸ are each —F or each are —H, and wherein Z² is —OH or —OS(O)₂CH₃,

and R¹-R⁵, R⁹-R¹⁴, X¹-X⁴ and Z¹ are defined as in general formula (I) including the substitutions and preferred definitions.

A further preferred aspect of the present invention relates to compounds of general formula (Ia) and salts and solvates thereof, wherein R⁶ and R⁷ form together a cyclic residue including the carbon atom to which they are bound and wherein the cyclic residue is cyclopropyl, and wherein R⁸ is —H,

and wherein Z¹ is selected from —H, —CH₃ and —CF₃, and wherein Z² is —OH or —OS(O)₂CH₃, and R¹-R⁵, R⁹-R¹⁴ and X¹-X⁴ are defined as in general formula (I) including the substitutions and preferred definitions.

A further preferred aspect of the present invention relates to compounds of general formula (I) and salts and solvates thereof, wherein R¹ is selected from residues as contained in the general definition of R¹, which contain four or more, preferably six or more and even more preferably seven or more carbon atoms,

and wherein R¹ contains no heteroatom,

and wherein R¹ is even more preferably selected from cyclic, bicyclic and tricyclic structures,

and wherein R¹ is even more preferably selected from cyclohexyl, norbornyl, bicyclooctyl, bicyclononyl, methylbicyclononyl, tricyclodecyl and adamantyl,

and wherein R¹ is most preferably adamantyl,

and R²-R⁸, R¹¹-R¹⁷, X¹-X⁴, Z¹ and Z² are defined as in general formula (I) including the substitutions and preferred definitions.

A further preferred aspect of the present invention relates to compounds of general formula (I) and salts and solvates thereof, wherein R¹ is selected from residues as contained in the general definition of R¹, which contain four or more, preferably six or more and even more preferably seven or more carbon atoms,

and wherein R¹ contains one or more preferably one to two heteroatoms independently selected from O, S and N in replacement of a carbon atom contained in R¹,

and wherein R¹ is even more preferably selected from cyclic, bicyclic and tricyclic structures, or wherein R¹ is selected from residues containing cyclic, bicyclic and tricyclic structures, and wherein R¹ is even more preferably selected from tetrahydropyranyl, N-methylpiperidinyl, morpholinyl, 4-oxocyclohexyl, azabicycloheptyl, N-methylazabicycloheptyl, oxa-azabicycloheptyl, N-methyldiazabicycloheptyl, azabicyclooctyl, diazabicyclooctyl, N-methyldiazabicyclooctyl, oxa-azabicyclooctyl, azabicyclononyl, aza-adamantyl and —O(adamantyl),

and wherein R¹ is most preferably tetrahydropyranyl, N-methylpiperidinyl, morpholinyl, 4-oxocyclohexyl, azabicyclooctyl, aza-adamantyl and —O(adamantyl),

and R²-R¹⁷, X¹-X⁴, Z¹ and Z² are defined as in general formula (I) including the substitutions and preferred definitions.

In a certain embodiment, the present invention relates to compounds of general formula (I) and salts and solvates thereof, wherein R¹ is adamantyl,

and wherein Z¹ and Z² are defined as in general formula (I), including general formula (Ia), general formula (Ib) and general formula (Ic), including the substitutions and preferred definitions,

and wherein R¹⁵ is defined as in general formula (Ia) including the substitutions and preferred definitions, and wherein R¹⁶ and R¹⁷ are defined as in general formula (Ib) including the substitutions and preferred definitions,

and wherein R²-R⁸, R¹¹-R¹⁴ and X¹-X⁴ are defined as in general formula (I) including the substitutions and preferred definitions, and wherein the compounds share the following structure (I-1):

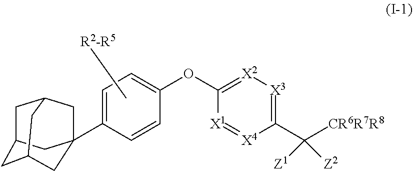

and wherein the compounds of structure (I-1) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPF-0014, XPF-0042, XPF-0070, XPF-0182, XPF-0210, XPF-0266, XPF-0434, XPF-0476, XPF-0504, XPF-0518, XPF-0630, XPF-1162, XPF-1190, XPF-1330, XPF-1554, XPF-1596, XPF-1624, XPF-2242, XPF-2244, XPF-2245, XPF-2247, XPF-2251, XPF-2252, XPF-2253 and XPF-2254.

In a further certain embodiment, the present invention relates to compounds of general formula (I) and salts and solvates thereof, and wherein R¹ is defined as in general formula (I) including the substitutions and preferred definitions, wherein R¹ is selected from cyclic, bicyclic and tricyclic structures, and wherein R¹ contains six or more carbon atoms, which are optionally independently replaced by a heteroatom selected from O, S and N as defined in general formula (I),

wherein R⁶ is defined as in general formula (I) including the substitutions and preferred definitions, wherein R⁶ is different from —H, optionally with the additional proviso that R⁶ is different from —CH₃,

and wherein Z¹ and Z² are defined as in general formula (I), including general formula (Ia), general formula (Ib) and general formula (Ic), including the substitutions and preferred definitions,

and wherein R¹⁵ is defined as in general formula (Ia) including the substitutions and preferred definitions, and wherein R¹⁶ and R¹⁷ are defined as in general formula (Ib) including the substitutions and preferred definitions,

and wherein R²-R⁵, R⁷-R¹⁴ and X¹-X⁴ are defined as in general formula (I) including the substitutions and preferred definitions,

and wherein the compounds share the following structure (I-2):

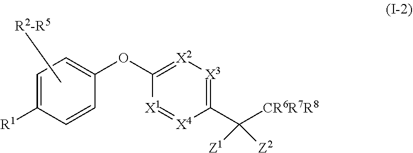

and wherein the compounds of structure (I-2) are—particularly without the additional proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPF-0042, XPF-0062, XPF-0063, XPF-0064, XPF-0065, XPF-0070, XPF-0202, XPF-0205, XPF-0210, XPF-0230, XPF-0426, XPF-0429, XPF-0434, XPF-0454, XPF-0469, XPF-0476, XPF-0496, XPF-0504, XPF-0518, XPF-0630, XPF-1162, XPF-1182, XPF-1185, XPF-1190, XPF-1196, XPF-1322, XPF-1325, XPF-1330, XPF-1546, XPF-1549, XPF-1554, XPF-1588, XPF-1596, XPF-1602, XPF-1616, XPF-1624, XPF-2241, XPF-2242, XPF-2243, XPF-2244, XPF-2245, XPF-2246, XPF-2247, XPF-2248, XPF-2249, XPF-2250, XPF-2251, XPF-2252, XPF-2253 and XPF-2254.

In a further certain embodiment, the present invention relates to compounds of general formula (I) and salts and solvates thereof, and wherein R¹ is defined as in general formula (I) including the substitutions and preferred definitions, wherein R¹ is selected from cyclic, bicyclic and tricyclic structures, and wherein R¹ contains six or more carbon atoms, which are optionally independently replaced by a heteroatom selected from O, S and N as defined in general formula (I),

wherein R⁸ is defined as in general formula (I) including the substitutions and preferred definitions, wherein R⁸ is different from —H, optionally with the additional proviso that R⁸ is different from —CH₃,

and wherein Z¹ and Z² are defined as in general formula (I), including general formula (Ia), general formula (Ib) and general formula (Ic), including the substitutions and preferred definitions,

and wherein R¹⁵ is defined as in general formula (Ia) including the substitutions and preferred definitions, and wherein R¹⁶ and R¹⁷ are defined as in general formula (Ib) including the substitutions and preferred definitions,

and wherein R²-R⁷, R⁹-R¹⁴ and X¹-X⁴ are defined as in general formula (I) including the substitutions and preferred definitions,

and wherein the compounds share the following structure (I-3):

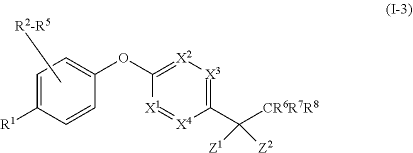

and wherein the compounds of structure (I-3) are—particularly without the additional proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPF-0062, XPF-0063, XPF-0064, XPF-0065, XPF-0070, XPF-0202, XPF-0205, XPF-0210, XPF-0230, XPF-0426, XPF-0429, XPF-0434, XPF-0454, XPF-0469, XPF-0476, XPF-0496, XPF-0504, XPF-0518, XPF-0630, XPF-1182, XPF-1185, XPF-1190, XPF-1196, XPF-1322, XPF-1325, XPF-1330, XPF-1546, XPF-1549, XPF-1554, XPF-1588, XPF-1596, XPF-1602, XPF-1616, XPF-1624, XPF-2241, XPF-2242, XPF-2243, XPF-2244, XPF-2245, XPF-2246, XPF-2247, XPF-2248, XPF-2249, XPF-2250, XPF-2251, XPF-2252, XPF-2253 and XPF-2254.

In a further certain embodiment, the present invention relates to compounds of general formula (I) and salts and solvates thereof, wherein R⁶, R⁷ and R⁸ are each —H, and wherein X¹ is CR¹¹, X² is CR¹², X³ is CR¹³ and X⁴ is CR¹⁴,

and wherein R¹ is defined as in general formula (I) including the substitutions and preferred definitions, wherein R¹ is selected from cyclic, bicyclic and tricyclic structures, and wherein R¹ contains six or more carbon atoms, which are optionally independently replaced by a heteroatom selected from O, S and N as defined in general formula (I), with the proviso that R¹ including any substituent contains no or one heteroatom selected from O, S, N,

and wherein Z¹ and Z² are defined as in general formula (I), including general formula (Ia), general formula (Ib) and general formula (Ic), including the substitutions and preferred definitions,

and wherein R¹⁵ is defined as in general formula (Ia) including the substitutions and preferred definitions, and wherein R¹⁶ and R¹⁷ are defined as in general formula (Ib) including the substitutions and preferred definitions,

and wherein R²-R⁵ and R⁹-R¹⁴ are defined as in general formula (I) including the substitutions and preferred definitions,

and wherein the compounds share the following structure (I-4):

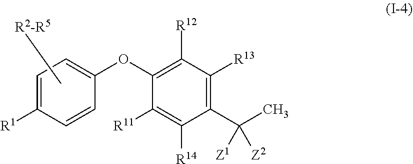

and wherein the compounds of structure (I-4) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast and cancer of the neuroendocrine system.

Examples are compounds XPF-0006, XPF-0014, XPF-0174 and XPF-0182, XPF-0258, XPF-0266.

In a further certain embodiment, the present invention relates to compounds of general formula (Ia) and salts and solvates thereof, wherein Z² is —OR¹⁵ and R¹⁵ is —H, and wherein R⁶, R⁷ and R⁸ are each —F,

and wherein Z¹ is defined as in general formula (Ia) including the substitutions and preferred definitions, optionally with the proviso that Z¹ is different from —CF₃,

and wherein R¹-R⁵, R⁹-R¹⁴ and X¹-X⁴ are defined as in general formula (I) including the substitutions and preferred definitions,

and wherein the compounds share the following structure (Ia-1):

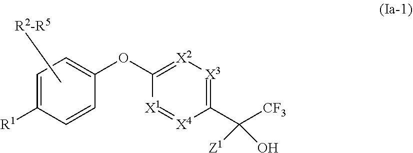

and wherein the compounds of structure (Ia-1) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPF-0057, XPF-0058, XPF-0062, XPF-0063, XPF-0064, XPF-0065, XPF-0070, XPF-0169, XPF-0170, XPF-0174, XPF-0182, XPF-0202, XPF-0205, XPF-0210, XPF-0230, XPF-0630, XPF-1178, XPF-1182, XPF-1185, XPF-1190, XPF-1322, XPF-1325, XPF-1330, XPF-2241, XPF-2242, XPF-2243, XPF-2244, XPF-2248, XPF-2251 and XPF-2252.

In a further certain embodiment, the present invention relates to compounds of general formula (Ia) and salts and solvates thereof, wherein Z¹ is cyclopropyl,

and wherein R¹ is defined as in general formula (I) including the substitutions and preferred definitions, optionally with the proviso that R¹ is different from —CF₃ and —CHF₂,

and wherein Z² and R¹⁵ are defined as in general formula (Ia) including the substitutions and preferred definitions,

and wherein R²-R¹⁴ and X¹-X⁴ are defined as in general formula (I) including the substitutions and preferred definitions,

and wherein the compounds share the following structure (Ia-2):

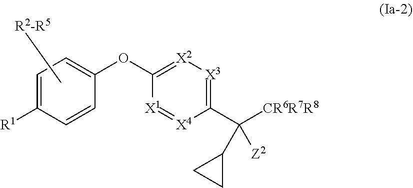

and wherein the compounds of structure (Ia-2) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, ovaries, and cancer of the neuroendocrine system.

Examples are compounds XPF-0202, XPF-0205, XPF-0210, XPF-1322, XPF-1325 and XPF-1330.

In a further certain embodiment, the present invention relates to compounds of general formula (Ia) and salts and solvates thereof, wherein R⁶ and R⁷ form together a cyclic residue including the carbon atom to which they are bound, and wherein the cyclic residue is C₃ cycloalkyl, i.e. cyclopropyl,

and wherein Z¹, Z² and R¹⁵ are defined as in general formula (Ia) including the substitutions and preferred definitions,

and wherein R¹-R⁵, R⁸-R¹⁴ and X¹-X⁴ are defined as in general formula (I) including the substitutions and preferred definitions,

and wherein the compounds share the following structure (Ia-3):

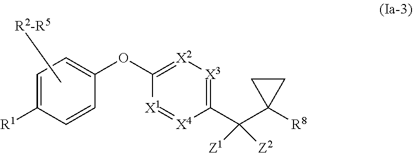

and wherein the compounds of structure (Ia-3) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, ovaries, and cancer of the neuroendocrine system.

Examples are compounds XPF-0042, XPF-0202, XPF-0205, XPF-0210, XPF-1162, XPF-1322, XPF-1325 and XPF-1330.

In a further certain embodiment, the present invention relates to compounds of general formula (Ia) and salts and solvates thereof, wherein R⁶, R⁷ and R⁸ are each —F,

and wherein Z¹ is defined as in general formula (Ia) including the substitutions and preferred definitions, optionally with the proviso that Z¹ is different from —CF₃,

and wherein Z² and R¹⁵ are defined as in general formula (Ia) including the substitutions and preferred definitions,

and wherein R¹-R⁵, R⁹-R¹⁴ and X¹-X⁴ are defined as in general formula (I) including the substitutions and preferred definitions,

and wherein the compounds share the following structure (Ia-4):

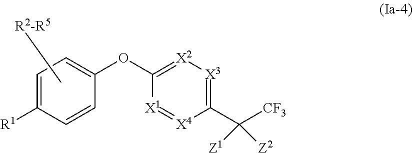

and wherein the compounds of structure (Ia-4) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPF-0057, XPF-0058, XPF-0062, XPF-0063, XPF-0064, XPF-0065, XPF-0070, XPF-0169, XPF-0170, XPF-0174, XPF-0182, XPF-0202, XPF-0205, XPF-0210, XPF-0230, XPF-0630, XPF-1178, XPF-1182, XPF-1185, XPF-1190, XPF-1196, XPF-1322, XPF-1325, XPF-1330, XPF-2241, XPF-2242, XPF-2243, XPF-2244, XPF-2248, XPF-2251 and XPF-2252.

In a further certain embodiment, the present invention relates to compounds of general formula (Ib) and salts and solvates thereof, wherein Z¹ and Z² are together ═NR¹⁶, and wherein R⁶, R⁷ and R⁸ are each —F,

and wherein R¹ is defined as in general formula (I) including the substitutions and preferred definitions, optionally with the proviso that R¹ is different from —CF₃,

and wherein R¹⁶ is defined as in general formula (Ib) including the substitutions and preferred definitions,

and wherein R²-R⁵, R⁹-R¹⁴ and X¹-X⁴ are defined as in general formula (I) including the substitutions and preferred definitions,

and wherein the compounds share the following structure (Ib-1):

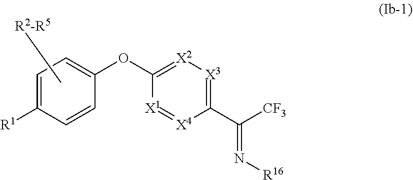

and wherein the compounds of structure (Ib-1) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

Examples are compounds XPF-0454, XPF-0469, XPF-0476, XPF-1588, XPF-1596, XPF-1602 and XPF-2249.

In a further certain embodiment, the present invention relates to compounds of general formula (Ib) and salts and solvates thereof, wherein Z¹ and Z² are together zwitterionic ═N^{[+]}R¹⁷O^{[-]},

and wherein R¹⁷ is defined as in general formula (Ib) including the substitutions and preferred definitions,

and wherein R¹-R¹⁴ and X¹-X⁴ are defined as in general formula (I) including the substitutions and preferred definitions,

and wherein the compounds share the following structure (Ib-2):

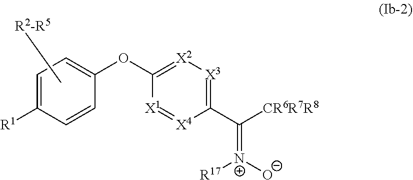

and wherein the compounds of structure (Ib-2) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, and cancer of the neuroendocrine system.

Examples are compounds XPF-0496, XPF-0504, XPF-1616 and XPF-1624.

In a further certain embodiment, the present invention relates to compounds of general formula (Ib) and salts and solvates thereof, wherein Z¹ and Z² are together zwitterionic ═N^{[+]}R¹⁷O^{[-]},

and wherein R⁶, R⁷ and R⁸ are each —F,

and wherein R¹⁷ is defined as in general formula (Ib) including the substitutions and preferred definitions,

and wherein R¹-R⁵, R⁹-R¹⁴ and X¹-X⁴ are defined as in general formula (I) including the substitutions and preferred definitions,

and wherein the compounds share the following structure (Ib-3):

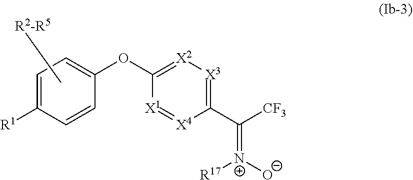

and wherein the compounds of structure (Ib-3) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, and cancer of the neuroendocrine system.

Examples are compounds XPF-0496, XPF-0504, XPF-1616 and XPF-1624.

In a further certain embodiment, the present invention relates to compounds of general formula (Ib) and salts and solvates thereof, wherein Z¹ and Z² are together ═O, and wherein R⁶, R⁷ and R⁸ are each —F,

and wherein R¹ is defined as in general formula (I) including the substitutions and preferred definitions, optionally with the proviso that R¹ is different from —CH₃ and —OCH₃,

and wherein R²-R⁵, R⁹-R¹⁴ and X¹-X⁴ are defined as in general formula (I) including the substitutions and preferred definitions,

and wherein the compounds share the following structure (Ib-4):

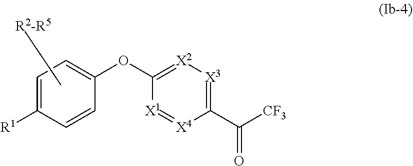

and wherein the compounds of structure (Ib-4) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, and cancer of the neuroendocrine system.

Examples are compounds XPF-0421, XPF-0422, XPF-0426, XPF-0429, XPF-0434, XPF-1541, XPF-1542, XPF-1546, XPF-1549, XPF-1554, XPF-2245, XPF-2246, XPF-2247, XPF-2250, XPF-2253 and XPF-2254.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions,

and wherein R⁶, R⁷ and R⁸ are each —F,

and wherein R¹-R⁵, R⁹-R¹⁴ and X¹-X⁴ are defined as in general formula (I) including the substitutions and preferred definitions,

and wherein the compounds share the following structure (Ic-1):

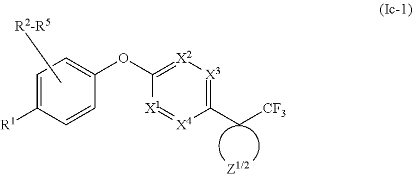

and wherein the compounds of structure (Ic-1) are preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

An example is compound XPF-0518.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions, and wherein the said cyclic residue is selected from three-membered rings and four-membered rings,

and wherein R⁸ is defined as in general formula (I) including the substitutions and preferred definitions, optionally with the proviso that R⁸ is different from —H,

and wherein R¹-R⁷, R⁹-R¹⁴ and X¹-X⁴ are defined as in general formula (I) including the substitutions and preferred definitions,

and wherein the compounds share the following structure (Ic-2):

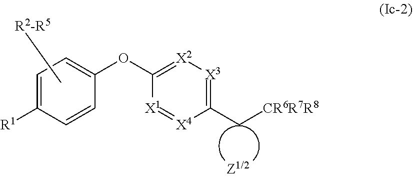

and wherein the compounds of structure (Ic-2) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

An example is compound XPF-0518.

In a further certain embodiment, the present invention relates to compounds of general formula (Ic) and salts and solvates thereof, wherein Z¹ and Z² form together a cyclic residue including the carbon atom to which they are bound, and wherein Z¹ and Z² are defined as in general formula (Ic) including the substitutions and preferred definitions, and wherein the said cyclic residue is selected from three-membered rings and four-membered rings, optionally with the proviso that the said cyclic residue is different from oxiranyl,

and wherein R¹-R¹⁴ and X¹-X⁴ are defined as in general formula (I) including the substitutions and preferred definitions,

and wherein the compounds share the following structure (Ic-3):

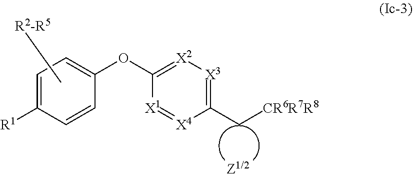

and wherein the compounds of structure (Ic-3) are—particularly without the proviso—preferred for use in human and veterinary medicine, in particular for the medical use described in the present invention, preferably for the use in immune system-related applications including immunotherapy and other immunotherapy methods as defined in the present invention, and in the treatment of immune system-related disorders, skin diseases, muscle diseases, hyperproliferative disorders and cancer including cancer of the haematopoietic and haematologic system such as leukemias and lymphomas, cancer of the skin, oral mucosa, tongue, lung, stomach, breast, cervix, ovaries, and cancer of the neuroendocrine system.

An example is compound XPF-0518.

In some embodiments, the following compounds shown in Table 1 to Table 3 are explicitly excluded from the scope of the invention: The compounds of Table 1 specifically indicated by CAS registry numbers have been identified by the inventors as state of the art. In embodiments where these compounds are encompassed by general formula (I) or any subgeneric formula as defined herein, they are explicitly excluded from the scope of the invention with regard to compound protection. To the best of the inventors' knowledge, these compounds are not known for any medical use. Thus, the invention encompasses any medical use for compounds of Table 1.

TABLE 1

| CAS | CAS | CAS | CAS | CAS |
|---|---|---|---|---|
| 1227-41-4 | 59803-52-0 | 98035-28-0 | 169245-61-8 | 866105-20-6 |
| 2093-04-1 | 61343-81-5 | 99433-47-3 | 169245-68-5 | 866114-07-0 |
| 2692-22-0 | 61658-96-6 | 100829-64-9 | 169245-69-6 | 869790-23-8 |
| 3093-75-2 | 61695-33-8 | 101585-43-7 | 169245-70-9 | 872976-92-6 |
| 5325-82-6 | 63845-18-1 | 101595-58-8 | 174669-93-3 | 887574-84-7 |
| 15484-64-7 | 64969-84-2 | 101945-81-7 | 183603-40-9 | 887574-95-0 |
| 15484-70-5 | 66473-68-5 | 105244-83-5 | 185697-26-1 | 887575-08-8 |
| 19366-31-5 | 68486-19-1 | 109502-81-0 | 189181-63-3 | 887575-25-9 |
| 19545-59-6 | 68548-65-2 | 110998-63-5 | 189181-92-8 | 895853-93-7 |
| 19545-62-1 | 68548-70-9 | 110998-74-8 | 189679-94-5 | 903288-98-2 |
| 19545-82-5 | 69064-79-5 | 114212-53-2 | 196604-17-8 | 903523-66-0 |
| 21302-54-5 | 69383-44-4 | 114371-94-7 | 199446-31-6 | 914637-09-5 |
| 22378-90-1 | 69591-15-7 | 114371-98-1 | 203866-25-5 | 915203-84-8 |
| 23713-94-2 | 70817-54-8 | 114371-99-2 | 205381-30-2 | 917611-21-3 |
| 24073-01-6 | 70945-85-6 | 117542-62-8 | 205381-32-4 | 934166-34-4 |
| 24073-18-5 | 70945-86-7 | 118308-60-4 | 213014-13-2 | 934195-71-8 |
| 24085-65-2 | 71815-31-1 | 118941-82-5 | 213744-59-3 | 938270-20-3 |
| 24190-42-9 | 72490-03-0 | 118941-83-6 | 257609-26-0 | 938292-64-9 |
| 30305-21-6 | 75090-68-5 | 118941-84-7 | 265648-04-2 | 952433-79-3 |
| 31477-15-3 | 75090-69-6 | 121771-44-6 | 287938-55-0 | 1019596-08-7 |
| 31477-17-5 | 77090-75-6 | 121913-57-3 | 287939-23-5 | 1038721-30-0 |
| 31477-19-7 | 77090-76-7 | 122085-50-1 | 287939-42-8 | 1041596-45-5 |
| 31477-41-5 | 77147-16-1 | 122085-51-2 | 287939-63-3 | 1054479-02-5 |
| 31477-42-6 | 80199-13-9 | 122106-59-6 | 287939-65-5 | 1054504-54-9 |
| 32565-50-7 | 80199-26-4 | 122141-69-9 | 287939-97-3 | 1054504-56-1 |
| 32728-37-3 | 80199-46-8 | 122568-91-6 | 287940-05-0 | 1054504-76-5 |
| 33012-20-3 | 80199-48-0 | 124747-22-4 | 287940-38-9 | 1055302-78-7 |
| 33565-80-9 | 80199-65-1 | 124747-23-5 | 287940-48-1 | 1055302-79-8 |
| 33565-81-0 | 80199-66-2 | 124747-24-6 | 287940-98-1 | 1055302-80-1 |
| 40843-21-8 | 80274-95-9 | 127948-27-0 | 287941-01-9 | 1055302-81-2 |
| 51318-79-7 | 82576-72-5 | 129400-86-8 | 287941-03-1 | 1055302-82-3 |
| 51318-80-0 | 83794-40-5 | 129400-92-6 | 287941-13-3 | 1068122-23-5 |
| 51338-19-3 | 83794-41-6 | 129643-32-9 | 287941-15-5 | 1068122-27-9 |
| 51363-31-6 | 84598-18-5 | 132529-76-1 | 292855-90-4 | 1068140-53-3 |
| 55814-55-6 | 84859-63-2 | 133447-18-4 | 293325-34-5 | 1068140-54-4 |
| 55814-56-7 | 84859-77-8 | 133595-88-7 | 312583-56-5 | 1068140-55-5 |
| 55814-57-8 | 85013-43-0 | 133748-96-6 | 332010-55-6 | 1068140-57-7 |
| 55814-58-9 | 85013-47-4 | 134822-96-1 | 345943-60-4 | 1071966-64-7 |
| 55814-67-0 | 85015-91-4 | 135533-56-1 | 378187-46-3 | 1072087-22-9 |
| 55814-70-5 | 85015-92-5 | 135937-16-5 | 459125-44-1 | 1072135-43-3 |
| 55814-71-6 | 85016-11-1 | 136100-38-4 | 459125-48-5 | 1072836-78-2 |
| 56595-28-9 | 86286-00-2 | 136943-50-5 | 606966-76-1 | 1097700-67-8 |
| 56718-33-3 | 86431-63-2 | 143213-44-9 | 606966-77-2 | 1098377-95-7 |
| 57148-30-8 | 86431-64-3 | 144742-60-9 | 643745-70-4 | 1126632-98-1 |
| 57148-33-1 | 86538-21-8 | 148254-66-4 | 709677-29-2 | 1126633-16-6 |
| 57945-73-0 | 86896-98-2 | 149993-89-5 | 717914-07-3 | 1126633-17-7 |
| 57945-74-1 | 86896-99-3 | 149993-96-4 | 726151-45-7 | 1126633-19-9 |
| 57945-75-2 | 87294-12-0 | 150607-84-4 | 741240-27-7 | 1126633-20-2 |
| 57945-76-3 | 87309-89-5 | 153821-80-8 | 767289-70-3 | 1129251-89-3 |
| 57945-77-4 | 88113-16-0 | 161759-38-2 | 774448-90-1 | 1130877-62-1 |
| 57945-78-5 | 91069-34-0 | 164517-87-7 | 808168-37-8 | 1156737-29-9 |
| 57945-79-6 | 92552-10-8 | 167026-55-3 | 808168-38-9 | 1156738-38-3 |
| 57945-80-9 | 93008-44-7 | 169243-90-7 | 808168-41-4 | 1182747-05-2 |
| 57945-81-0 | 93291-44-2 | 169243-91-8 | 816418-04-9 | 1189339-42-1 |
| 57945-82-1 | 93291-45-3 | 169245-42-5 | 847951-23-9 | 1195556-55-8 |
| 57945-83-2 | 93434-70-9 | 169245-43-6 | 855272-33-2 | 1198164-69-0 |
| 57945-84-3 | 93652-10-9 | 169245-44-7 | 855937-78-9 | 1198164-70-3 |
| 57945-85-4 | 94402-61-6 | 169245-45-8 | 857617-73-3 | 1198164-75-8 |
| 57945-86-5 | 94996-30-2 | 169245-56-1 | 857986-52-8 | 1198164-76-9 |
| 58291-19-3 | 97631-88-4 | 169245-57-2 | 860580-10-5 | 1198164-77-0 |
| 58291-48-8 | 98035-27-9 | 169245-60-7 | 866105-18-2 | 1198166-35-6 |
| 1423374-11-1 | 2247022-78-0 | 2323566-55-6 | 2323566-53-4 | 2323565-95-1 |
| CAS | CAS | CAS | CAS | CAS |
| 1198166-36-7 | 1554658-68-2 | 2082660-49-7 | 2213467-75-3 | 2218417-73-1 |
| 1198166-41-4 | 1556627-26-9 | 2082660-57-7 | 2213467-76-4 | 2218421-23-7 |
| 1198166-42-5 | 1607436-59-8 | 2082660-73-7 | 2213467-78-6 | 2218421-25-9 |
| 1198166-43-6 | 1608475-97-3 | 2082660-85-1 | 2213467-79-7 | 2218421-26-0 |
| 1198166-54-9 | 1609018-10-1 | 2082661-01-4 | 2213467-80-0 | 2218421-28-2 |
| 1198166-56-1 | 1609018-11-2 | 2082661-11-6 | 2213467-82-2 | 2218421-29-3 |
| 1198166-57-2 | 1609018-12-3 | 2082661-19-4 | 2213467-83-3 | 2218421-33-9 |
| 1198166-62-9 | 1609018-17-8 | 2082661-35-4 | 2216766-12-8 | 2218421-35-1 |
| 1198166-63-0 | 1609018-18-9 | 2082661-47-8 | 2216766-16-2 | 2218421-36-2 |
| 1198166-64-1 | 1609018-19-0 | 2082661-67-2 | 2216766-17-3 | 2218421-42-0 |
| 1198168-51-2 | 1609018-21-4 | 2082661-71-8 | 2216766-18-4 | 2218421-43-1 |
| 1198168-54-5 | 1609018-22-5 | 2082661-81-0 | 2216766-19-5 | 2218421-67-9 |
| 1198168-57-8 | 1609018-23-6 | 2082661-95-6 | 2216766-21-9 | 2218421-69-1 |
| 1198168-76-1 | 1609018-24-7 | 2082661-97-8 | 2216766-29-7 | 2218421-84-0 |
| 1198168-99-8 | 1609018-29-2 | 2082662-02-8 | 2218414-32-3 | 2218421-99-7 |
| 1198169-02-6 | 1609018-30-5 | 2082662-04-0 | 2218414-34-5 | 2218422-03-6 |
| 1198169-05-9 | 1609018-35-0 | 2082662-08-4 | 2218414-35-6 | 2218422-11-6 |
| 1198169-17-3 | 1609018-36-1 | 2082662-29-9 | 2218414-39-0 | 2218422-12-7 |
| 1198169-33-3 | 1609018-37-2 | 2082662-49-3 | 2218414-42-5 | 2218422-16-1 |
| 1198169-65-1 | 1609018-38-3 | 2082662-51-7 | 2218414-43-6 | 2218422-20-7 |
| 1198169-73-1 | 1609018-43-0 | 2082662-55-1 | 2218414-46-9 | 2218422-22-9 |
| 1242172-33-3 | 1609018-44-1 | 2082662-57-3 | 2218414-65-2 | 2218422-23-0 |
| 1242172-35-5 | 1609018-45-2 | 2082662-70-0 | 2218414-68-5 | 2218422-24-1 |
| 1242172-69-5 | 1609018-46-3 | 2082662-75-5 | 2218414-83-4 | 2218422-26-3 |
| 1242172-70-8 | 1609018-47-4 | 2082662-77-7 | 2218414-97-0 | 2218422-27-4 |
| 1307868-52-5 | 1609019-18-2 | 2082662-83-5 | 2218415-32-6 | 2218422-40-1 |
| 1310726-03-4 | 1609019-20-6 | 2082662-89-1 | 2218415-41-7 | 2218422-42-3 |
| 1334922-19-8 | 1609019-31-9 | 2082662-97-1 | 2218415-45-1 | 2218422-44-5 |
| 1334922-50-7 | 1609019-32-0 | 2082663-07-6 | 2218415-46-2 | 2218422-47-8 |
| 1334922-56-3 | 1609019-33-1 | 2082663-96-3 | 2218415-51-9 | 2222300-58-3 |
| 1334923-29-3 | 1609019-44-4 | 2082664-04-6 | 2218415-52-0 | 2222300-59-4 |
| 1351463-21-2 | 1609134-09-9 | 2082664-10-4 | 2218415-55-3 | 2226670-33-1 |
| 1355071-46-3 | 1612165-18-0 | 2082664-26-2 | 2218415-56-4 | 2226889-13-8 |
| 1355071-61-2 | 1612165-26-0 | 2082664-32-0 | 2218415-57-5 | 2229853-57-8 |
| 1357298-23-7 | 1612764-03-0 | 2082664-40-0 | 2218415-59-7 | 2241854-16-8 |
| 1361005-77-7 | 1612764-05-2 | 2082664-44-4 | 2218415-60-0 | 2241854-17-9 |
| 1361968-47-9 | 1612764-06-3 | 2082664-46-6 | 2218415-67-7 | 2241854-18-0 |
| 1361968-57-1 | 1612764-12-1 | 2082664-52-4 | 2218415-75-7 | 2241854-19-1 |
| 1367221-74-6 | 1622156-57-3 | 2082664-56-8 | 2218415-78-0 | 2241854-20-4 |
| 1378618-71-3 | 1622156-71-1 | 2082664-66-0 | 2218415-79-1 | 2241854-22-6 |
| 1403682-02-9 | 1627579-39-8 | 2082664-68-2 | 2218415-86-0 | 2241854-28-2 |
| 1422261-85-5 | 1670226-83-1 | 2082664-72-8 | 2218415-87-1 | 2241854-29-3 |
| 1430748-31-4 | 1695558-01-0 | 2095852-83-6 | 2218415-88-2 | 2241854-30-6 |
| 1439936-19-2 | 1799905-85-3 | 2098671-39-5 | 2218415-89-3 | 2241854-33-9 |
| 1439936-35-2 | 1801443-91-3 | 2098887-26-2 | 2218415-90-6 | 2241854-34-0 |
| 1439936-46-5 | 1801443-93-5 | 2098889-51-9 | 2218415-91-7 | 2241854-35-1 |
| 1439936-65-8 | 1801444-07-4 | 2126941-46-4 | 2218415-93-9 | 2241854-36-2 |
| 1440059-15-3 | 1801444-10-9 | 2126941-47-5 | 2218416-28-3 | 2247022-72-4 |
| 1440542-90-4 | 1801444-11-0 | 2126941-48-6 | 2218416-31-8 | 2248431-38-9 |
| 1448769-77-4 | 1801444-14-3 | 2126941-52-2 | 2218416-38-5 | 2259694-79-4 |
| 1465781-10-5 | 1801444-15-4 | 2126941-53-3 | 2218416-50-1 | 2259694-80-7 |
| 1476112-34-1 | 1809098-73-4 | 2128650-58-6 | 2218416-62-5 | 2290506-46-4 |
| 1487158-11-1 | 1835278-40-4 | 2138864-11-4 | 2218416-66-9 | 2290506-47-5 |
| 1491329-66-8 | 1835278-57-3 | 2138864-29-4 | 2218416-73-8 | 2290506-53-3 |
| 1544563-12-3 | 1897386-13-8 | 2172931-50-7 | 2218416-75-0 | |
| 1546175-36-3 | 1922959-46-3 | 2176456-51-0 | 2218416-76-1 | |
| 1546175-39-6 | 1949801-39-1 | 2176457-02-4 | 2218416-83-0 | |
| 1547800-75-8 | 1949801-48-2 | 2213467-62-8 | 2218416-85-2 | |
| 1549125-36-1 | 1949801-49-3 | 2213467-65-1 | 2218416-86-3 | |
| 1552596-24-3 | 2081130-42-7 | 2213467-68-4 | 2218417-28-6 | |
| 1553935-56-0 | 2082660-47-5 | 2213467-74-2 | 2218417-44-6 | |
| 2323565-93-9 | 2307450-68-4 | 2306121-67-3 | 2290506-54-4 | |

The compounds of Table 2 specifically indicated by CAS registry numbers have been identified by the inventors as state of the art. In embodiments, where these compounds are encompassed by general formula (I) or any subgeneric formula as defined herein, they are explicitly excluded from the scope of the invention with regard to compound protection. To the best of the inventors' knowledge, these compounds are not known for any medical use as defined in the invention. Thus, the compounds of Table 2 are explicitly included into the scope of the invention with regard to medical use as defined herein, particularly in the treatment of non-malignant or malignant hyperproliferative diseases.

TABLE 2

| CAS | CAS | CAS | CAS | CAS | CAS | CAS | CAS | CAS | CAS |
|---|---|---|---|---|---|---|---|---|---|
| 94402-73-0 | 189874-89-3 | 213691-48-6 | 216304-45-9 | 917613-60-6 | 952433-30-6 | 1071966-77-2 | 1098438-25-5 | 1358753-79-3 | 1440054- |
| | | | | | | | | | 01-2 |
| 98054-56-9 | 189875-55-6 | 213691-49-7 | 235441-42-6 | 934195-72-9 | 952433-31-7 | 1071966-82-9 | 1098438-34-6 | 1403681-56-0 | 1440054- |
| | | | | | | | | | 48-7 |
| 120848-98-8 | 212187-25-2 | 213691-57-7 | 235441-44-8 | 935985-47-0 | 1058157-88-2 | 1098436-04-4 | 1098438-56-2 | 1403681-61-7 | |
| 172931-40-7 | 213691-41-9 | 216304-42-6 | 669014-87-3 | 935995-53-2 | 1058158-37-4 | 1098438-20-0 | 1098439-16-7 | 1430410-29-9 | |
| 874489-03-9 | 874518-46-4 | 181144-96-7 | 181144-95-6 | 874518-46-4 | 874489-03-9 | 181144-97-8 | 1622156-72-2 | 1542139-55-8 | |

The compounds of Table 3 specifically indicated by CAS registry numbers have been identified by the inventors as state of the art. In embodiments, where these compounds are encompassed by general formula (I) or any subgeneric formula as defined herein, they are explicitly excluded from the scope of the invention with regard to compound protection. Further, these compounds are, to the best of the inventors' knowledge, known for a medical use, which in some embodiments may be encompassed by a medical use as defined herein. Thus, the compounds of Table 3 may be explicitly excluded from the scope of the invention with regard to compound protection and with regard to certain medical use in some embodiments as defined herein.

TABLE 3

| CAS | Reference |
|---|---|
| 54916-28-8 | WO2005044263 A1 |
| 94064-20-7 | WO9920263 A1 |
| 219930-72-0 | WO2003007955 A2 |
| 220088-55-1 | WO2003007955 A2 |
| 223769-08-2 | WO9920263 A1 |
| 270260-17-8 | JP2000143650 A |
| 270260-18-9 | JP2000143650 A |
| 270260-19-0 | JP2000143650 A |
| 380184-29-2 | US20090163545 A1 |
| 620628-15-1 | WO2003091252 A1 |
| 844635-75-2 | US20050038051 A1 |
| 851461-55-7 | 10.1016/j.bmcl.2005.02.038 |
| 872977-07-6 | 10.1002/alca.200590220 |
| 947548-36-9 | WO2007096647 A2 |
| 947548-42-7 | WO2007096647 A2 |
| 1012035-42-5 | WO2008030892 A2 |
| 1012035-44-7 | WO2008030892 A2 |
| 1012035-47-0 | WO2008030892 A2 |
| 1300560-28-4 | US20110105482 A1 |
| 1300560-34-2 | US20110105482 A1 |
| 1300561-63-0 | US20110105482 A1 |
| 1300561-66-3 | US20110105482 A1 |
| 1417988-65-8 | WO2013004190 A1 |
| 1417988-68-1 | WO2013004190 A1 |
| 1417988-94-3 | WO2013004190 A1/EP2786982 |
| 1417988-95-4 | WO2013004190 A1/EP2786982 |
| 1417988-96-5 | WO2013004190 A1/EP2786982 |
| 1417988-97-6 | WO2013004190 A1/EP2786982 |
| 1417988-98-7 | WO2013004190 A1/EP2786982 |
| 1417988-99-8 | WO2013004190 A1/EP2786982 |
| 1417989-93-5 | WO2013004190 A1/EP2786982 |
| 1417989-96-8 | WO2013004190 A1 |
| 1417990-21-6 | WO2013004190 A1/EP2786982 |
| 1417990-22-7 | WO2013004190 A1/EP2786982 |
| 1417990-23-8 | WO2013004190 A1/EP2786982 |
| 1417990-24-9 | WO2013004190 A1/EP2786982 |
| 1417990-25-0 | WO2013004190 A1/EP2786982 |
| 1417990-26-1 | WO2013004190 A1/EP2786982 |
| 1440541-27-4 | EP2789607 |
| 1440541-74-1 | EP2789607 |
| 2095854-10-5 | WO2018144870 A1 |
| 2306183-58-2 | CN109134336 A |

Specific examples of compounds falling under the scope of formula (I) are shown in Table 4 to Table 28. Intermediates are denoted as “XPF-T”.

TABLE 4

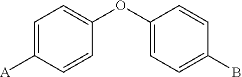

| | B | | | |
|---|---|---|---|---|
| A | 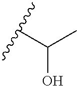 [SMILES: *C(C)O] | 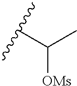 [SMILES: *C(C)OS(C)(=O)=O] | 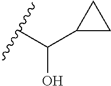 [SMILES: *C(O)C1CC1] | 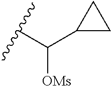 [SMILES: *C(OS(C)(=O)=O)C1CC1] |
| 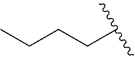 [SMILES: *CCCC] | XPF-0001 | XPF-0015 | XPF-0029 | XPF-0043 |
| 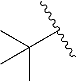 [SMILES: *C(C)(C)C] | XPF-0002 | XPF-0016 | XPF-0030 | XPF-0044 |
| 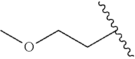 [SMILES: *CCOC] | XPF-0003 | XPF-0017 | XPF-0031 | XPF-0045 |
| 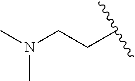 [SMILES: *CCN(C)C] | XPF-0004 | XPF-0018 | XPF-0032 | XPF-0046 |
| 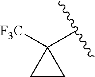 [SMILES: *C1(C(F)(F)F)CC1] | XPF-0005 | XPF-0019 | XPF-0033 | XPF-0047 |
| 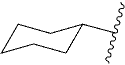 [SMILES: *C1CCCCC1] | XPF-0006 | XPF-0020 | XPF-0034 | XPF-0048 |
| 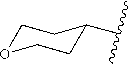 [SMILES: *C1CCOCC1] | XPF-0007 | XPF-0021 | XPF-0035 | XPF-0049 |
| 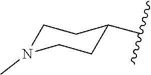 [SMILES: *C1CCN(C)CC1] | XPF-0008 | XPF-0022 | XPF-0036 | XPF-0050 |
| 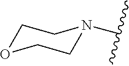 [SMILES: *N1CCOCC1] | XPF-0009 | XPF-0023 | XPF-0037 | XPF-0051 |
| 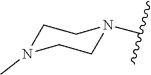 [SMILES: *N1CCN(C)CC1] | XPF-0010 | XPF-0024 | XPF-0038 | XPF-0052 |
| 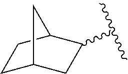 | XPF-0011 | XPF-0025 | XPF-0039 | XPF-0053 |
| 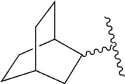 | XPF-0012 | XPF-0026 | XPF-0040 | XPF-0054 |
| 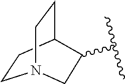 | XPF-0013 | XPF-0027 | XPF-0041 | XPF-0055 |
| 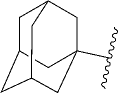 [SMILES: *C12CC3CC(CC(C3)C1)C2] | XPF-0014 | XPF-0028 | XPF-0042 | XPF-0056 |
| | B | | | |
| A | 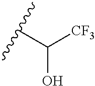 [SMILES: *C(O)C(F)(F)F] | 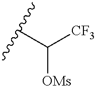 [SMILES: *C(OS(C)(=O)=O)C(F)(F)F] | 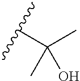 [SMILES: *C(C)(C)O] | 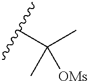 [SMILES: *C(C)(C)OS(C)(=O)=O] |
| 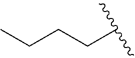 [SMILES: *CCCC] | XPF-0057 | XPF-0071 | XPF-0085 | XPF-0099 |
| 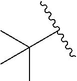 [SMILES: *C(C)(C)C] | XPF-0058 | XPF-0072 | XPF-0086 | XPF-0100 |
| 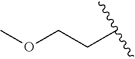 [SMILES: *CCOC] | XPF-0059 | XPF-0073 | XPF-0087 | XPF-0101 |
| 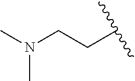 [SMILES: *CCN(C)C] | XPF-0060 | XPF-0074 | XPF-0088 | XPF-0102 |
| 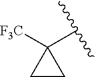 [SMILES: *C1(C(F)(F)F)CC1] | XPF-0061 | XPF-0075 | XPF-0089 | XPF-0103 |
| 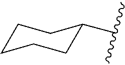 [SMILES: *C1CCCCC1] | XPF-0062 | XPF-0076 | XPF-0090 | XPF-0104 |
| 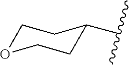 [SMILES: *C1CCOCC1] | XPF-0063 | XPF-0077 | XPF-0091 | XPF-0105 |
| 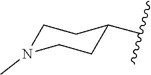 [SMILES: *C1CCN(C)CC1] | XPF-0064 | XPF-0078 | XPF-0092 | XPF-0106 |
| 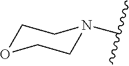 [SMILES: *N1CCOCC1] | XPF-0065 | XPF-0079 | XPF-0093 | XPF-0107 |
| 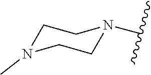 [SMILES: *N1CCN(C)CC1] | XPF-0066 | XPF-0080 | XPF-0094 | XPF-0108 |
| 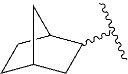 | XPF-0067 | XPF-0081 | XPF-0095 | XPF-0109 |
| 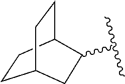 | XPF-0068 | XPF-0082 | XPF-0096 | XPF-0110 |
| 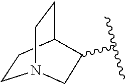 | XPF-0069 | XPF-0083 | XPF-0097 | XPF-0111 |
| 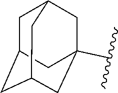 [SMILES: *C12CC3CC(CC(C3)C1)C2] | XPF-0070 | XPF-0084 | XPF-0098 | XPF-0112 |

| | B | | |
|---|---|---|---|
| A | 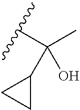 [SMILES: *C(C)(O)C1CC1] | 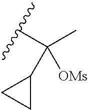 [SMILES: *C(C)(OS(C)(=O)=O)C1CC1] | 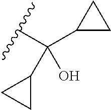 [SMILES: *C(O)(C1CC1)C1CC1] |
| 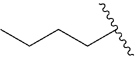 [SMILES: *CCCC] | XPF-0113 | XPF-0127 | XPF-0141 |
| 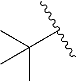 [SMILES: *C(C)(C)C] | XPF-0114 | XPF-0128 | XPF-0142 |
| 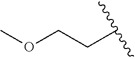 [SMILES: *CCOC] | XPF-0115 | XPF-0129 | XPF-0143 |
| 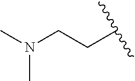 [SMILES: *CCN(C)C] | XPF-0116 | XPF-0130 | XPF-0144 |
| 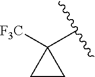 [SMILES: *C1(C(F)(F)F)CC1] | XPF-0117 | XPF-0131 | XPF-0145 |
| 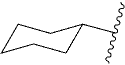 [SMILES: *C1CCCCC1] | XPF-0118 | XPF-0132 | XPF-0146 |
| 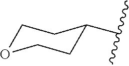 [SMILES: *C1CCOCC1] | XPF-0119 | XPF-0133 | XPF-0147 |
| 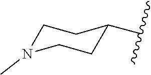 [SMILES: *C1CCN(C)CC1] | XPF-0120 | XPF-0134 | XPF-0148 |
| 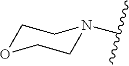 [SMILES: *N1CCOCC1] | XPF-0121 | XPF-0135 | XPF-0149 |
| 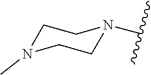 [SMILES: *N1CCN(C)CC1] | XPF-0122 | XPF-0136 | XPF-0150 |
| 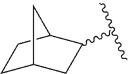 | XPF-0123 | XPF-0137 | XPF-0151 |
| 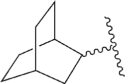 | XPF-0124 | XPF-0138 | XPF-0152 |
| 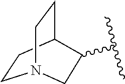 | XPF-0125 | XPF-0139 | XPF-0153 |
| 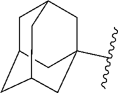 [SMILES: *C12CC3CC(CC(C3)C1)C2] | XPF-0126 | XPF-0140 | XPF-0154 |

The above table constitutes an individualized description of each of the specifically indicated compounds therein as well as their salts and solvates.

TABLE 5

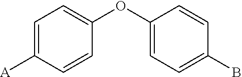

| | B | | | |
|---|---|---|---|---|
| A | 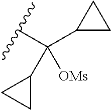 [SMILES: *C(OS(C)(=O)=O)(C1CC1)C1CC1] | 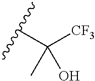 [SMILES: *C(C)(O)C(F)(F)F] | 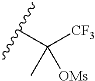 [SMILES: *C(C)(OS(C)(=O)=O)C(F)(F)F] | 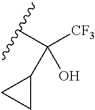 [SMILES: *C(O)(C1CC1)C(F)(F)F] |
| 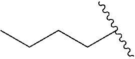 [SMILES: *CCCC] | XPF-0155 | XPF-0169 | XPF-0183 | XPF-0197 |
| 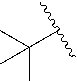 [SMILES: *C(C)(C)C] | XPF-0156 | XPF-0170 | XPF-0184 | XPF-0198 |
| 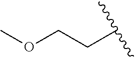 [SMILES: *CCOC] | XPF-0157 | XPF-0171 | XPF-0185 | XPF-0199 |
| 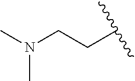 [SMILES: *CCN(C)C] | XPF-0158 | XPF-0172 | XPF-0186 | XPF-0200 |
| 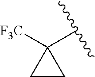 [SMILES: *C1(C(F)(F)F)CC1] | XPF-0159 | XPF-0173 | XPF-0187 | XPF-0201 |
| 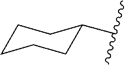 [SMILES: *C1CCCCC1] | XPF-0160 | XPF-0174 | XPF-0188 | XPF-0202 |
| 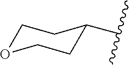 [SMILES: *C1CCOCC1] | XPF-0161 | XPF-0175 | XPF-0189 | XPF-0203 |
| 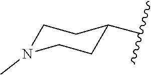 [SMILES: *C1CCN(C)CC1] | XPF-0162 | XPF-0176 | XPF-0190 | XPF-0204 |
| 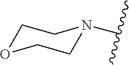 [SMILES: *N1CCOCC1] | XPF-0163 | XPF-0177 | XPF-0191 | XPF-0205 |
| 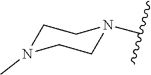 [SMILES: *N1CCN(C)CC1] | XPF-0164 | XPF-0178 | XPF-0192 | XPF-0206 |
| 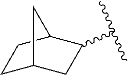 | XPF-0165 | XPF-0179 | XPF-0193 | XPF-0207 |
| 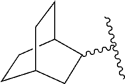 | XPF-0166 | XPF-0180 | XPF-0194 | XPF-0208 |
| 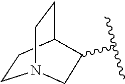 | XPF-0167 | XPF-0181 | XPF-0195 | XPF-0209 |
| 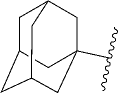 [SMILES: *C12CC3CC(CC(C3)C1)C2] | XPF-0168 | XPF-0182 | XPF-0196 | XPF-0210 |

| | B | | |
|---|---|---|---|
| A | 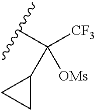 [SMILES: *C(OS(C)(=O)=O)(C1CC1)C(F)(F)F] | 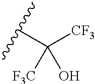 [SMILES: *C(O)(C(F)(F)F)C(F)(F)F] | 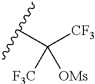 [SMILES: *C(OS(C)(=O)=O)(C(F)(F)F)C(F)(F)F] |
| 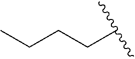 [SMILES: *CCCC] | XPF-0211 | XPF-0225 | XPF-0239 |
| 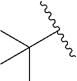 [SMILES: *C(C)(C)C] | XPF-0212 | XPF-0226 | XPF-0240 |
| 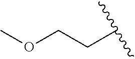 [SMILES: *CCOC] | XPF-0213 | XPF-0227 | XPF-0241 |
| 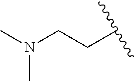 [SMILES: *CCN(C)C] | XPF-0214 | XPF-0228 | XPF-0242 |
| 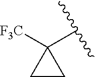 [SMILES: *C1(C(F)(F)F)CC1] | XPF-0215 | XPF-0229 | XPF-0243 |
| 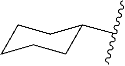 [SMILES: *C1CCCCC1] | XPF-0216 | XPF-0230 | XPF-0244 |
| 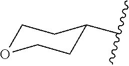 [SMILES: *C1CCOCC1] | XPF-0217 | XPF-0231 | XPF-0245 |
| 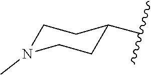 [SMILES: *C1CCN(C)CC1] | XPF-0218 | XPF-0232 | XPF-0246 |
| 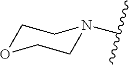 [SMILES: *N1CCOCC1] | XPF-0219 | XPF-0233 | XPF-0247 |
| 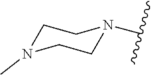 [SMILES: *N1CCN(C)CC1] | XPF-0220 | XPF-0234 | XPF-0248 |
| 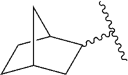 | XPF-0221 | XPF-0235 | XPF-0249 |
| 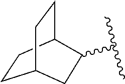 | XPF-0222 | XPF-0236 | XPF-0250 |
| 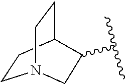 | XPF-0223 | XPF-0237 | XPF-0251 |
| 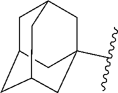 [SMILES: *C12CC3CC(CC(C3)C1)C2] | XPF-0224 | XPF-0238 | XPF-0252 |

The above table constitutes an individualized description of each of the specifically indicated compounds therein as well as their salts and solvates.

TABLE 6

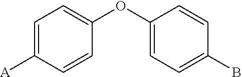

| | B | | | |
|---|---|---|---|---|
| A | 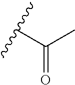 [SMILES: *C(C)=O] | 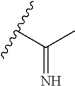 [SMILES: *C(C)=N] | 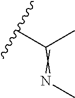 [SMILES: *C(C)=NC] | 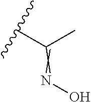 [SMILES: *C(C)=NO] |
| 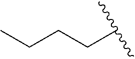 [SMILES: *CCCC] | XPF-0253 | XPF-0267 | XPF-0281 | XPF-0295 |
| 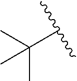 [SMILES: *C(C)(C)C] | XPF-0254 | XPF-0268 | XPF-0282 | XPF-0296 |
| 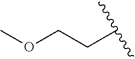 [SMILES: *CCOC] | XPF-0255 | XPF-0269 | XPF-0283 | XPF-0297 |
| 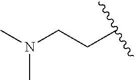 [SMILES: *CCN(C)C] | XPF-0256 | XPF-0270 | XPF-0284 | XPF-0298 |
| 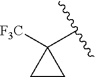 [SMILES: *C1(C(F)(F)F)CC1] | XPF-0257 | XPF-0271 | XPF-0285 | XPF-0299 |
| 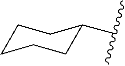 [SMILES: *C1CCCCC1] | XPF-0258 | XPF-0272 | XPF-0286 | XPF-0300 |
| 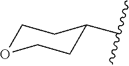 [SMILES: *C1CCOCC1] | XPF-0259 | XPF-0273 | XPF-0287 | XPF-0301 |
| 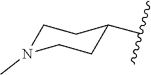 [SMILES: *C1CCN(C)CC1] | XPF-0260 | XPF-0274 | XPF-0288 | XPF-0302 |
| 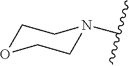 [SMILES: *N1CCOCC1] | XPF-0261 | XPF-0275 | XPF-0289 | XPF-0303 |
| 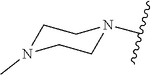 [SMILES: *N1CCN(C)CC1] | XPF-0262 | XPF-0276 | XPF-0290 | XPF-0304 |
| 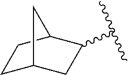 | XPF-0263 | XPF-0277 | XPF-0291 | XPF-0305 |
| 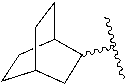 | XPF-0264 | XPF-0278 | XPF-0292 | XPF-0306 |
| 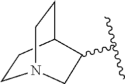 | XPF-0265 | XPF-0279 | XPF-0293 | XPF-0307 |
| 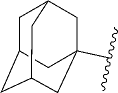 [SMILES: *C12CC3CC(CC(C3)C1)C2] | XPF-0266 | XPF-0280 | XPF-0294 | XPF-0308 |
| | B | | | |
| A | 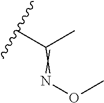 [SMILES: *C(C)=NOC] | 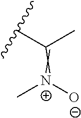 [SMILES: *C(C)=[N+](C)[O-]] | 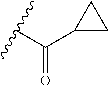 [SMILES: *C(=O)C1CC1] | 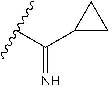 [SMILES: *C(=N)C1CC1] |
| 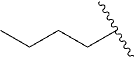 [SMILES: *CCCC] | XPF-0309 | XPF-0323 | XPF-0337 | XPF-0351 |
| 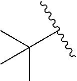 [SMILES: *C(C)(C)C] | XPF-0310 | XPF-0324 | XPF-0338 | XPF-0352 |
| 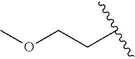 [SMILES: *CCOC] | XPF-0311 | XPF-0325 | XPF-0339 | XPF-0353 |
| 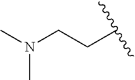 [SMILES: *CCN(C)C] | XPF-0312 | XPF-0326 | XPF-0340 | XPF-0354 |
| 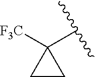 [SMILES: *C1(C(F)(F)F)CC1] | XPF-0313 | XPF-0327 | XPF-0341 | XPF-0355 |
| 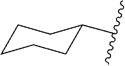 [SMILES: *C1CCCCC1] | XPF-0314 | XPF-0328 | XPF-0342 | XPF-0356 |
| 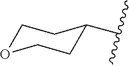 [SMILES: *C1CCOCC1] | XPF-0315 | XPF-0329 | XPF-0343 | XPF-0357 |
| 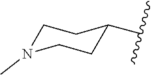 [SMILES: *C1CCN(C)CC1] | XPF-0316 | XPF-0330 | XPF-0344 | XPF-0358 |
| 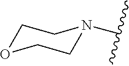 [SMILES: *N1CCOCC1] | XPF-0317 | XPF-0331 | XPF-0345 | XPF-0359 |
| 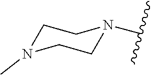 [SMILES: *N1CCN(C)CC1] | XPF-0318 | XPF-0332 | XPF-0346 | XPF-0360 |
| 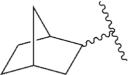 | XPF-0319 | XPF-0333 | XPF-0347 | XPF-0361 |
| 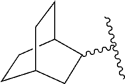 | XPF-0320 | XPF-0334 | XPF-0348 | XPF-0362 |
| 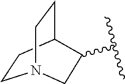 | XPF-0321 | XPF-0335 | XPF-0349 | XPF-0363 |
| 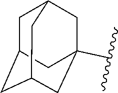 [SMILES: *C12CC3CC(CC(C3)C1)C2] | XPF-0322 | XPF-0336 | XPF-0350 | XPF-0364 |

| | B | | |
|---|---|---|---|
| A | 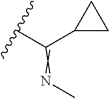 [SMILES: *C(=NC)C1CC1] | 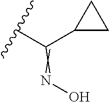 [SMILES: *C(=NO)C1CC1] | 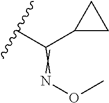 [SMILES: *C(=NOC)C1CC1] |
| 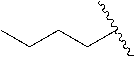 [SMILES: *CCCC] | XPF-0365 | XPF-0379 | XPF-0393 |
| 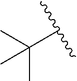 [SMILES: *C(C)(C)C] | XPF-0366 | XPF-0380 | XPF-0394 |
| 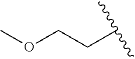 [SMILES: *CCOC] | XPF-0367 | XPF-0381 | XPF-0395 |
| 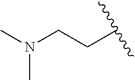 [SMILES: *CCN(C)C] | XPF-0368 | XPF-0382 | XPF-0396 |
| 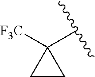 [SMILES: *C1(C(F)(F)F)CC1] | XPF-0369 | XPF-0383 | XPF-0397 |
| 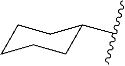 [SMILES: *C1CCCCC1] | XPF-0370 | XPF-0384 | XPF-0398 |
| 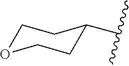 [SMILES: *C1CCOCC1] | XPF-0371 | XPF-0385 | XPF-0399 |
| 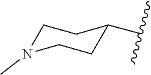 [SMILES: *C1CCN(C)CC1] | XPF-0372 | XPF-0386 | XPF-0400 |
| 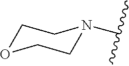 [SMILES: *N1CCOCC1] | XPF-0373 | XPF-0387 | XPF-0401 |
| 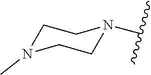 [SMILES: *N1CCN(C)CC1] | XPF-0374 | XPF-0388 | XPF-0402 |
| 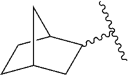 | XPF-0375 | XPF-0389 | XPF-0403 |
| 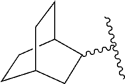 | XPF-0376 | XPF-0390 | XPF-0404 |
| 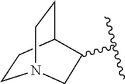 | XPF-0377 | XPF-0391 | XPF-0405 |
| 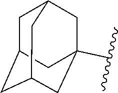 [SMILES: *C12CC3CC(CC(C3)C1)C2] | XPF-0378 | XPF-0392 | XPF-0406 |

The above table constitutes an individualized description of each of the specifically indicated compounds therein as well as their salts and solvates.

TABLE 7

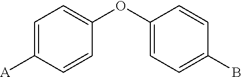

| | B | | | |
|---|---|---|---|---|
| A | 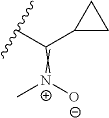 [SMILES: *C(C1CC1)=[N+](C)[O-]] | 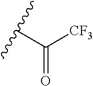 [SMILES: *C(=O)C(F)(F)F] | 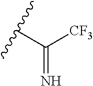 [SMILES: *C(=N)C(F)(F)F] | 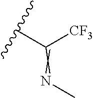 [SMILES: *C(=NC)C(F)(F)F] |
| 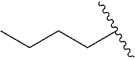 [SMILES: *CCCC] | XPF-0407 | XPF-0421 | XPF-0435 | XPF-0449 |
| 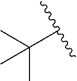 [SMILES: *C(C)(C)C] | XPF-0408 | XPF-0422 | XPF-0436 | XPF-0450 |
| 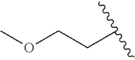 [SMILES: *CCOC] | XPF-0409 | XPF-0423 | XPF-0437 | XPF-0451 |
| 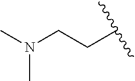 [SMILES: *CCN(C)C] | XPF-0410 | XPF-0424 | XPF-0438 | XPF-0452 |
| 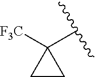 [SMILES: *C1(C(F)(F)F)CC1] | XPF-0411 | XPF-0425 | XPF-0439 | XPF-0453 |
| 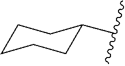 [SMILES: *C1CCCCC1] | XPF-0412 | XPF-0426 | XPF-0440 | XPF-0454 |
| 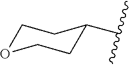 [SMILES: *C1CCOCC1] | XPF-0413 | XPF-0427 | XPF-0441 | XPF-0455 |
| 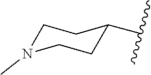 [SMILES: *C1CCN(C)CC1] | XPF-0414 | XPF-0428 | XPF-0442 | XPF-0456 |
| 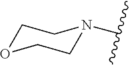 [SMILES: *N1CCOCC1] | XPF-0415 | XPF-0429 | XPF-0443 | XPF-0457 |
| 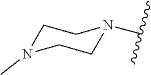 [SMILES: *N1CCN(C)CC1] | XPF-0416 | XPF-0430 | XPF-0444 | XPF-0458 |
| 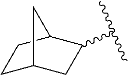 | XPF-0417 | XPF-0431 | XPF-0445 | XPF-0459 |
| 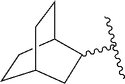 | XPF-0418 | XPF-0432 | XPF-0446 | XPF-0460 |
| 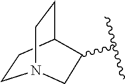 | XPF-0419 | XPF-0433 | XPF-0447 | XPF-0461 |
| 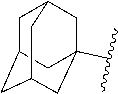 [SMILES: *C12CC3CC(CC(C3)C1)C2] | XPF-0420 | XPF-0434 | XPF-0448 | XPF-0462 |

| | B | | |
|---|---|---|---|
| A | 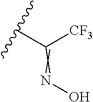 [SMILES: *C(=NO)C(F)(F)F] | 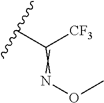 [SMILES: *C(=NOC)C(F)(F)F] | 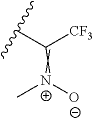 [SMILES: *C(=[N+](C)[O-])C(F)(F)F] |
| 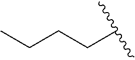 [SMILES: *CCCC] | XPF-0463 | XPF-0477 | XPF-0491 |
| 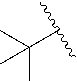 [SMILES: *C(C)(C)C] | XPF-0464 | XPF-0478 | XPF-0492 |
| 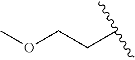 [SMILES: *CCOC] | XPF-0465 | XPF-0479 | XPF-0493 |
| 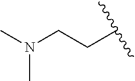 [SMILES: *CCN(C)C] | XPF-0466 | XPF-0480 | XPF-0494 |
| 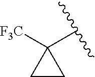 [SMILES: *C1(C(F)(F)F)CC1] | XPF-0467 | XPF-0481 | XPF-0495 |
| 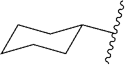 [SMILES: *C1CCCCC1] | XPF-0469 | XPF-0482 | XPF-0496 |
| 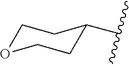 [SMILES: *C1CCOCC1] | XPF-0468 | XPF-0483 | XPF-0497 |
| 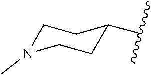 [SMILES: *C1CCN(C)CC1] | XPF-0470 | XPF-0484 | XPF-0498 |
| 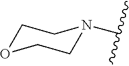 [SMILES: *N1CCOCC1] | XPF-0471 | XPF-0485 | XPF-0499 |
| 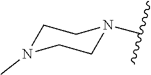 [SMILES: *N1CCN(C)CC1] | XPF-0472 | XPF-0486 | XPF-0500 |
| 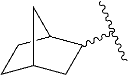 | XPF-0473 | XPF-0487 | XPF-0501 |
| 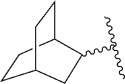 | XPF-0474 | XPF-0488 | XPF-0502 |
| 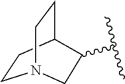 | XPF-0475 | XPF-0489 | XPF-0503 |
| 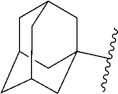 [SMILES: *C12CC3CC(CC(C3)C1)C2] | XPF-0476 | XPF-0490 | XPF-0504 |

The above table constitutes an individualized description of each of the specifically indicated compounds therein as well as their salts and solvates.

TABLE 8

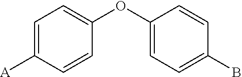

| | B | | | |
|---|---|---|---|---|
| A | 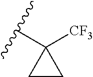 [SMILES: *C1(C(F)(F)F)CC1] | 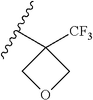 [SMILES: *C1(C(F)(F)F)COC1] | 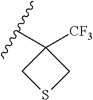 [SMILES: *C1(C(F)(F)F)CSC1] | 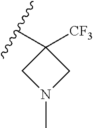 [SMILES: *C1(C(F)(F)F)CN(C)C1] |
| 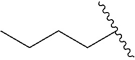 [SMILES: *CCCC] | XPF-0505 | XPF-0519 | XPF-0533 | XPF-0547 |
| 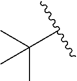 [SMILES: *C(C)(C)C] | XPF-0506 | XPF-0520 | XPF-0534 | XPF-0548 |
| 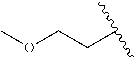 [SMILES: *CCOC] | XPF-0507 | XPF-0521 | XPF-0535 | XPF-0549 |
| 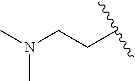 [SMILES: *CCN(C)C] | XPF-0508 | XPF-0522 | XPF-0536 | XPF-0550 |
| 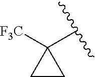 [SMILES: *C1(C(F)(F)F)CC1] | XPF-0509 | XPF-0523 | XPF-0537 | XPF-0551 |
| 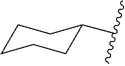 [SMILES: *C1CCCCC1] | XPF-0510 | XPF-0524 | XPF-0538 | XPF-0552 |
| 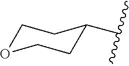 [SMILES: *C1CCOCC1] | XPF-0511 | XPF-0525 | XPF-0539 | XPF-0553 |
| 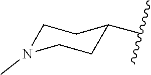 [SMILES: *C1CCN(C)CC1] | XPF-0512 | XPF-0526 | XPF-0540 | XPF-0554 |
| 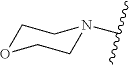 [SMILES: *N1CCOCC1] | XPF-0513 | XPF-0527 | XPF-0541 | XPF-0555 |
| 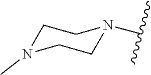 [SMILES: *N1CCN(C)CC1] | XPF-0514 | XPF-0528 | XPF-0542 | XPF-0556 |
| 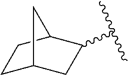 | XPF-0515 | XPF-0529 | XPF-0543 | XPF-0557 |
| 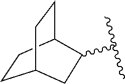 | XPF-0516 | XPF-0530 | XPF-0544 | XPF-0558 |
| 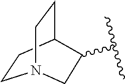 | XPF-0517 | XPF-0531 | XPF-0545 | XPF-0559 |
| 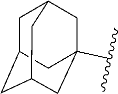 [SMILES: *C12CC3CC(CC(C3)C1)C2] | XPF-0518 | XPF-0532 | XPF-0546 | XPF-0560 |

The above table constitutes an individualized description of each of the specifically indicated compounds therein as well as their salts and solvates.

TABLE 9

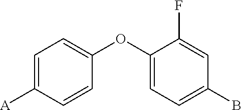

| | B | | | |
|---|---|---|---|---|
| A | 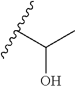 [SMILES: *C(C)O] | 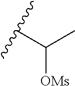 [SMILES: *C(C)OS(C)(=O)=O] | 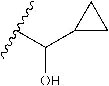 [SMILES: *C(O)C1CC1] | 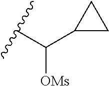 [SMILES: *C(OS(C)(=O)=O)C1CC1] |
| 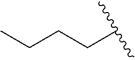 [SMILES: *CCCC] | XPF-0561 | XPF-0575 | XPF-0589 | XPF-0603 |
| 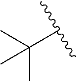 [SMILES: *C(C)(C)C] | XPF-0562 | XPF-0576 | XPF-0590 | XPF-0604 |
| 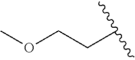 [SMILES: *CCOC] | XPF-0563 | XPF-0577 | XPF-0591 | XPF-0605 |
| 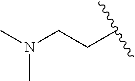 [SMILES: *CCN(C)C] | XPF-0564 | XPF-0578 | XPF-0592 | XPF-0606 |
| 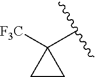 [SMILES: *C1(C(F)(F)F)CC1] | XPF-0565 | XPF-0579 | XPF-0593 | XPF-0607 |
| 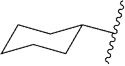 [SMILES: *C1CCCCC1] | XPF-0566 | XPF-0580 | XPF-0594 | XPF-0608 |
| 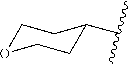 [SMILES: *C1CCOCC1] | XPF-0567 | XPF-0581 | XPF-0595 | XPF-0609 |
| 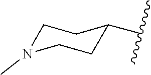 [SMILES: *C1CCN(C)CC1] | XPF-0568 | XPF-0582 | XPF-0596 | XPF-0610 |
| 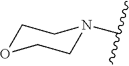 [SMILES: *N1CCOCC1] | XPF-0569 | XPF-0583 | XPF-0597 | XPF-0611 |
| 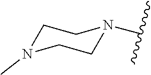 [SMILES: *N1CCN(C)CC1] | XPF-0570 | XPF-0584 | XPF-0598 | XPF-0612 |
| 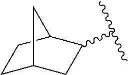 | XPF-0571 | XPF-0585 | XPF-0599 | XPF-0613 |
| 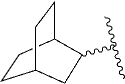 | XPF-0572 | XPF-0586 | XPF-0600 | XPF-0614 |
| 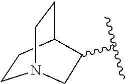 | XPF-0573 | XPF-0587 | XPF-0601 | XPF-0615 |
| 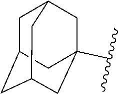 [SMILES: *C12CC3CC(CC(C3)C1)C2] | XPF-0574 | XPF-0588 | XPF-0602 | XPF-0616 |
| | B | | | |
| A | 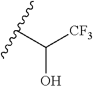 [SMILES: *C(O)C(F)(F)F] | 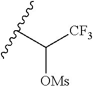 [SMILES: *C(OS(C)(=O)=O)C(F)(F)F] | 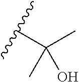 [SMILES: *C(C)(C)O] | 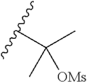 [SMILES: *C(C)(C)OS(C)(=O)=O] |
| 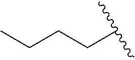 [SMILES: *CCCC] | XPF-0617 | XPF-0631 | XPF-0645 | XPF-0659 |
| 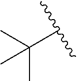 [SMILES: *C(C)(C)C] | XPF-0618 | XPF-0632 | XPF-0646 | XPF-0660 |
| 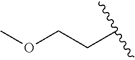 [SMILES: *CCOC] | XPF-0619 | XPF-0633 | XPF-0647 | XPF-0661 |
| 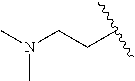 [SMILES: *CCN(C)C] | XPF-0620 | XPF-0634 | XPF-0648 | XPF-0662 |
| 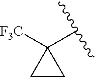 [SMILES: *C1(C(F)(F)F)CC1] | XPF-0621 | XPF-0635 | XPF-0649 | XPF-0663 |
| 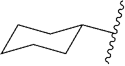 [SMILES: *C1CCCCC1] | XPF-0622 | XPF-0636 | XPF-0650 | XPF-0664 |
| 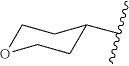 [SMILES: *C1CCOCC1] | XPF-0623 | XPF-0637 | XPF-0651 | XPF-0665 |
| 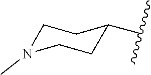 [SMILES: *C1CCN(C)CC1] | XPF-0624 | XPF-0638 | XPF-0652 | XPF-0666 |
| 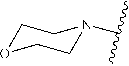 [SMILES: *N1CCOCC1] | XPF-0625 | XPF-0639 | XPF-0653 | XPF-0667 |
| 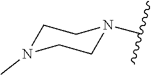 [SMILES: *N1CCN(C)CC1] | XPF-0626 | XPF-0640 | XPF-0654 | XPF-0668 |
| 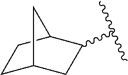 | XPF-0627 | XPF-0641 | XPF-0655 | XPF-0669 |
| 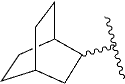 | XPF-0628 | XPF-0642 | XPF-0656 | XPF-0670 |
| 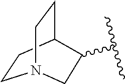 | XPF-0629 | XPF-0643 | XPF-0657 | XPF-0671 |
| 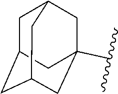 [SMILES: *C12CC3CC(CC(C3)C1)C2] | XPF-0630 | XPF-0644 | XPF-0658 | XPF-0672 |

| | B | | |
|---|---|---|---|
| A | 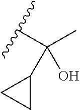 [SMILES: *C(C)(O)C1CC1] | 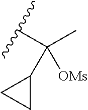 [SMILES: *C(C)(OS(C)(=O)=O)C1CC1] | 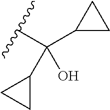 [SMILES: *C(O)(C1CC1)C1CC1] |
| 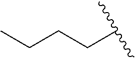 [SMILES: *CCCC] | XPF-0673 | XPF-0687 | XPF-0701 |
| 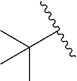 [SMILES: *C(C)(C)C] | XPF-0674 | XPF-0688 | XPF-0702 |
| 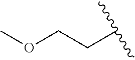 [SMILES: *CCOC] | XPF-0675 | XPF-0689 | XPF-0703 |
| 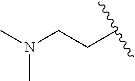 [SMILES: *CCN(C)C] | XPF-0676 | XPF-0690 | XPF-0704 |
| 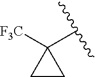 [SMILES: *C1(C(F)(F)F)CC1] | XPF-0677 | XPF-0691 | XPF-0705 |
| 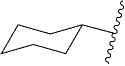 [SMILES: *C1CCCCC1] | XPF-0678 | XPF-0692 | XPF-0706 |
| 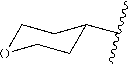 [SMILES: *C1CCOCC1] | XPF-0679 | XPF-0693 | XPF-0707 |
| 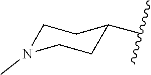 [SMILES: *C1CCN(C)CC1] | XPF-0680 | XPF-0694 | XPF-0708 |
| 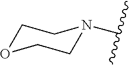 [SMILES: *N1CCOCC1] | XPF-0681 | XPF-0695 | XPF-0709 |
| 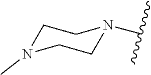 [SMILES: *N1CCN(C)CC1] | XPF-0682 | XPF-0696 | XPF-0710 |
| 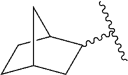 | XPF-0683 | XPF-0697 | XPF-0711 |
| 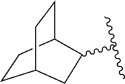 | XPF-0684 | XPF-0698 | XPF-0712 |
| 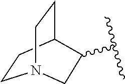 | XPF-0685 | XPF-0699 | XPF-0713 |
| 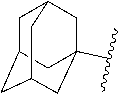 [SMILES: *C12CC3CC(CC(C3)C1)C2] | XPF-0686 | XPF-0700 | XPF-0714 |

The above table constitutes an individualized description of each of the specifically indicated compounds therein as well as their salts and solvates.

TABLE 10

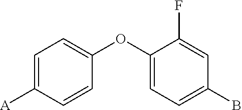

| | B | | | |
|---|---|---|---|---|
| A | 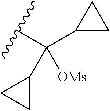 [SMILES: *C(OS(C)(=O)=O)(C1CC1)C1CC1] | 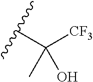 [SMILES: *C(C)(O)C(F)(F)F] | 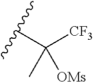 [SMILES: *C(C)(OS(C)(=O)=O)C(F)(F)F] | 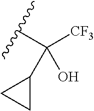 [SMILES: *C(O)(C1CC1)C(F)(F)F] |
| 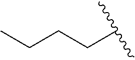 [SMILES: *CCCC] | XPF-0715 | XPF-0729 | XPF-0743 | XPF-0757 |
| 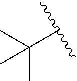 [SMILES: *C(C)(C)C] | XPF-0716 | XPF-0730 | XPF-0744 | XPF-0758 |
| 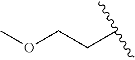 [SMILES: *CCOC] | XPF-0717 | XPF-0731 | XPF-0745 | XPF-0759 |
| 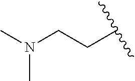 [SMILES: *CCN(C)C] | XPF-0718 | XPF-0732 | XPF-0746 | XPF-0760 |
| 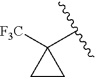 [SMILES: *C1(C(F)(F)F)CC1] | XPF-0719 | XPF-0733 | XPF-0747 | XPF-0761 |
| 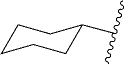 [SMILES: *C1CCCCC1] | XPF-0720 | XPF-0734 | XPF-0748 | XPF-0762 |
| 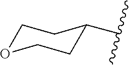 [SMILES: *C1CCOCC1] | XPF-0721 | XPF-0735 | XPF-0749 | XPF-0763 |
| 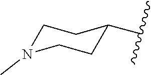 [SMILES: *C1CCN(C)CC1] | XPF-0722 | XPF-0736 | XPF-0750 | XPF-0764 |
| 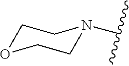 [SMILES: *N1CCOCC1] | XPF-0723 | XPF-0737 | XPF-0751 | XPF-0765 |
| 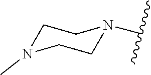 [SMILES: *N1CCN(C)CC1] | XPF-0724 | XPF-0738 | XPF-0752 | XPF-0766 |
| 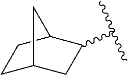 | XPF-0725 | XPF-0739 | XPF-0753 | XPF-0767 |
| 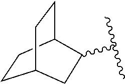 | XPF-0726 | XPF-0740 | XPF-0754 | XPF-0768 |
| 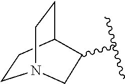 | XPF-0727 | XPF-0741 | XPF-0755 | XPF-0769 |
| 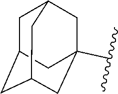 [SMILES: *C12CC3CC(CC(C3)C1)C2] | XPF-0728 | XPF-0742 | XPF-0756 | XPF-0770 |

| | B | | |
|---|---|---|---|
| A | 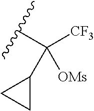 [SMILES: *C(OS(C)(=O)=O)(C1CC1)C(F)(F)F] | 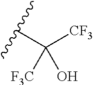 [SMILES: *C(O)(C(F)(F)F)C(F)(F)F] | 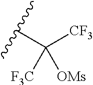 [SMILES: *C(OS(C)(=O)=O)(C(F)(F)F)C(F)(F)F] |
| 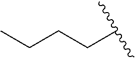 [SMILES: *CCCC] | XPF-0771 | XPF-0785 | XPF-0799 |
| 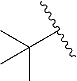 [SMILES: *C(C)(C)C] | XPF-0772 | XPF-0786 | XPF-0800 |
| 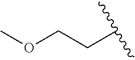 [SMILES: *CCOC] | XPF-0773 | XPF-0787 | XPF-0801 |
| 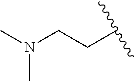 [SMILES: *CCN(C)C] | XPF-0774 | XPF-0788 | XPF-0802 |
| 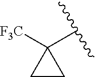 [SMILES: *C1(C(F)(F)F)CC1] | XPF-0775 | XPF-0789 | XPF-0803 |
| 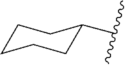 [SMILES: *C1CCCCC1] | XPF-0776 | XPF-0790 | XPF-0804 |
| 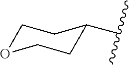 [SMILES: *C1CCOCC1] | XPF-0777 | XPF-0791 | XPF-0805 |
| 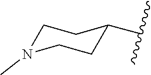 [SMILES: *C1CCN(C)CC1] | XPF-0778 | XPF-0792 | XPF-0806 |
| 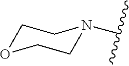 [SMILES: *N1CCOCC1] | XPF-0779 | XPF-0793 | XPF-0807 |
| 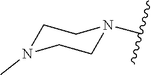 [SMILES: *N1CCN(C)CC1] | XPF-0780 | XPF-0794 | XPF-0808 |
| 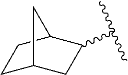 | XPF-0781 | XPF-0795 | XPF-0809 |
| 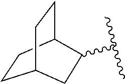 | XPF-0782 | XPF-0796 | XPF-0810 |
| 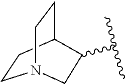 | XPF-0783 | XPF-0797 | XPF-0811 |
| 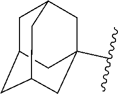 [SMILES: *C12CC3CC(CC(C3)C1)C2] | XPF-0784 | XPF-0798 | XPF-0812 |

The above table constitutes an individualized description of each of the specifically indicated compounds therein as well as their salts and solvates.

TABLE 11

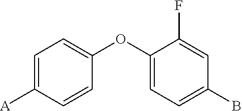

| | B | | | |
|---|---|---|---|---|
| A | 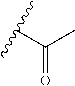 [SMILES: *C(C)=O] | 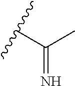 [SMILES: *C(C)=N] | 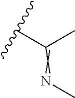 [SMILES: *C(C)=NC] | 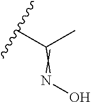 [SMILES: *C(C)=NO] |
| 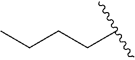 [SMILES: *CCCC] | XPF-0813 | XPF-0827 | XPF-0841 | XPF-0855 |
| 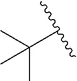 [SMILES: *C(C)(C)C] | XPF-0814 | XPF-0828 | XPF-0842 | XPF-0856 |
| 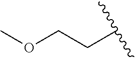 [SMILES: *CCOC] | XPF-0815 | XPF-0829 | XPF-0843 | XPF-0857 |
| 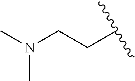 [SMILES: *CCN(C)C] | XPF-0816 | XPF-0830 | XPF-0844 | XPF-0858 |
| 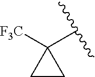 [SMILES: *C1(C(F)(F)F)CC1] | XPF-0817 | XPF-0831 | XPF-0845 | XPF-0859 |
| 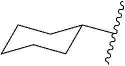 [SMILES: *C1CCCCC1] | XPF-0818 | XPF-0832 | XPF-0846 | XPF-0860 |
| 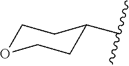 [SMILES: *C1CCOCC1] | XPF-0819 | XPF-0833 | XPF-0847 | XPF-0861 |
| 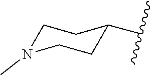 [SMILES: *C1CCN(C)CC1] | XPF-0820 | XPF-0834 | XPF-0848 | XPF-0862 |
| 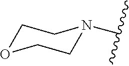 [SMILES: *N1CCOCC1] | XPF-0821 | XPF-0835 | XPF-0849 | XPF-0863 |
| 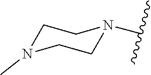 [SMILES: *N1CCN(C)CC1] | XPF-0822 | XPF-0836 | XPF-0850 | XPF-0864 |
| 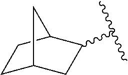 | XPF-0823 | XPF-0837 | XPF-0851 | XPF-0865 |
| 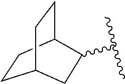 | XPF-0824 | XPF-0838 | XPF-0852 | XPF-0866 |
| 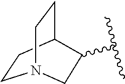 | XPF-0825 | XPF-0839 | XPF-0853 | XPF-0867 |
| 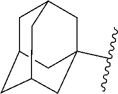 [SMILES: *C12CC3CC(CC(C3)C1)C2] | XPF-0826 | XPF-0840 | XPF-0854 | XPF-0868 |
| | B | | | |
| A | 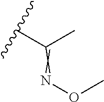 [SMILES: *C(C)=NOC] | 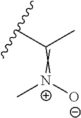 [SMILES: *C(C)=[N+](C)[O-]] | 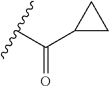 [SMILES: *C(=O)C1CC1] | 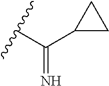 [SMILES: *C(=N)C1CC1] |
| 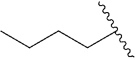 [SMILES: *CCCC] | XPF-0869 | XPF-0883 | XPF-0897 | XPF-0911 |
| 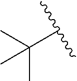 [SMILES: *C(C)(C)C] | XPF-0870 | XPF-0884 | XPF-0898 | XPF-0912 |
| 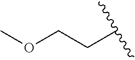 [SMILES: *CCOC] | XPF-0871 | XPF-0885 | XPF-0899 | XPF-0913 |
| 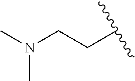 [SMILES: *CCN(C)C] | XPF-0872 | XPF-0886 | XPF-0900 | XPF-0914 |
| 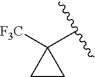 [SMILES: *C1(C(F)(F)F)CC1] | XPF-0873 | XPF-0887 | XPF-0901 | XPF-0915 |
| 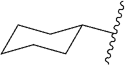 [SMILES: *C1CCCCC1] | XPF-0874 | XPF-0888 | XPF-0902 | XPF-0916 |
| 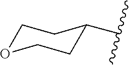 [SMILES: *C1CCOCC1] | XPF-0875 | XPF-0889 | XPF-0903 | XPF-0917 |
| 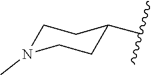 [SMILES: *C1CCN(C)CC1] | XPF-0876 | XPF-0890 | XPF-0904 | XPF-0918 |
| 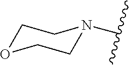 [SMILES: *N1CCOCC1] | XPF-0877 | XPF-0891 | XPF-0905 | XPF-0919 |
| 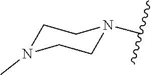 [SMILES: *N1CCN(C)CC1] | XPF-0878 | XPF-0892 | XPF-0906 | XPF-0920 |
| 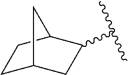 | XPF-0879 | XPF-0893 | XPF-0907 | XPF-0921 |
| 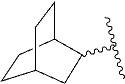 | XPF-0880 | XPF-0894 | XPF-0908 | XPF-0922 |
| 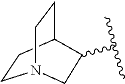 | XPF-0881 | XPF-0895 | XPF-0909 | XPF-0923 |
| 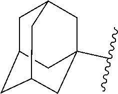 [SMILES: *C12CC3CC(CC(C3)C1)C2] | XPF-0882 | XPF-0896 | XPF-0910 | XPF-0924 |

| | B | | |
|---|---|---|---|
| A | 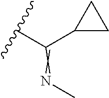 [SMILES: *C(=NC)C1CC1] | 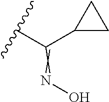 [SMILES: *C(=NO)C1CC1] | 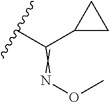 [SMILES: *C(=NOC)C1CC1] |
| 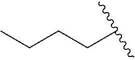 [SMILES: *CCCC] | XPF-0925 | XPF-0939 | XPF-0953 |
| 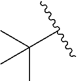 [SMILES: *C(C)(C)C] | XPF-0926 | XPF-0940 | XPF-0954 |
| 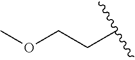 [SMILES: *CCOC] | XPF-0927 | XPF-0941 | XPF-0955 |
| 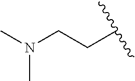 [SMILES: *CCN(C)C] | XPF-0928 | XPF-0942 | XPF-0956 |
| 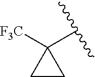 [SMILES: *C1(C(F)(F)F)CC1] | XPF-0929 | XPF-0943 | XPF-0957 |
| 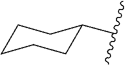 [SMILES: *C1CCCCC1] | XPF-0930 | XPF-0944 | XPF-0958 |
| 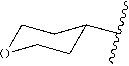 [SMILES: *C1CCOCC1] | XPF-0931 | XPF-0945 | XPF-0959 |
| 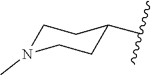 [SMILES: *C1CCN(C)CC1] | XPF-0932 | XPF-0946 | XPF-0960 |
| 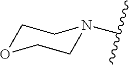 [SMILES: *N1CCOCC1] | XPF-0933 | XPF-0947 | XPF-0961 |
| 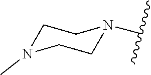 [SMILES: *N1CCN(C)CC1] | XPF-0934 | XPF-0948 | XPF-0962 |
| 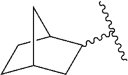 | XPF-0935 | XPF-0949 | XPF-0963 |
| 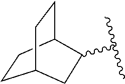 | XPF-0936 | XPF-0950 | XPF-0964 |
| 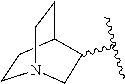 | XPF-0937 | XPF-0951 | XPF-0965 |
| 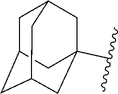 [SMILES: *C12CC3CC(CC(C3)C1)C2] | XPF-0938 | XPF-0952 | XPF-0966 |

The above table constitutes an individualized description of each of the specifically indicated compounds therein as well as their salts and solvates.

TABLE 12

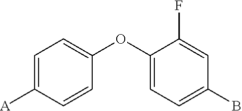

| | B | | | |
|---|---|---|---|---|
| A | 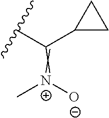 [SMILES: *C(C1CC1)=[N+](C)[O-]] | 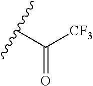 [SMILES: *C(=O)C(F)(F)F] | 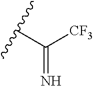 [SMILES: *C(=N)C(F)(F)F] | 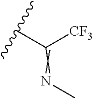 [SMILES: *C(=NC)C(F)(F)F] |
| 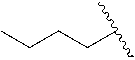 [SMILES: *CCCC] | XPF-0967 | XPF-0981 | XPF-0995 | XPF-1009 |
| 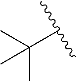 [SMILES: *C(C)(C)C] | XPF-0968 | XPF-0982 | XPF-0996 | XPF-1010 |
| 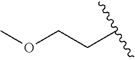 [SMILES: *CCOC] | XPF-0969 | XPF-0983 | XPF-0997 | XPF-1011 |
| 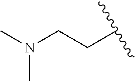 [SMILES: *CCN(C)C] | XPF-0970 | XPF-0984 | XPF-0998 | XPF-1012 |
| 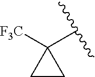 [SMILES: *C1(C(F)(F)F)CC1] | XPF-0971 | XPF-0985 | XPF-0999 | XPF-1013 |
| 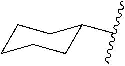 [SMILES: *C1CCCCC1] | XPF-0972 | XPF-0986 | XPF-1000 | XPF-1014 |
| 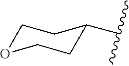 [SMILES: *C1CCOCC1] | XPF-0973 | XPF-0987 | XPF-1001 | XPF-1015 |
| 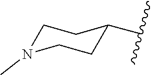 [SMILES: *C1CCN(C)CC1] | XPF-0974 | XPF-0988 | XPF-1002 | XPF-1016 |
| 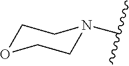 [SMILES: *N1CCOCC1] | XPF-0975 | XPF-0989 | XPF-1003 | XPF-1017 |
| 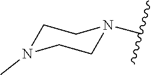 [SMILES: *N1CCN(C)CC1] | XPF-0976 | XPF-0990 | XPF-1004 | XPF-1018 |
| 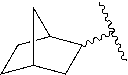 | XPF-0977 | XPF-0991 | XPF-1005 | XPF-1019 |
| 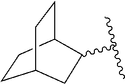 | XPF-0978 | XPF-0992 | XPF-1006 | XPF-1020 |
| 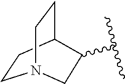 | XPF-0979 | XPF-0993 | XPF-1007 | XPF-1021 |
| 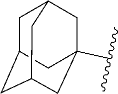 [SMILES: *C12CC3CC(CC(C3)C1)C2] | XPF-0980 | XPF-0994 | XPF-1008 | XPF-1022 |

| | B | | |
|---|---|---|---|
| A | 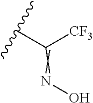 [SMILES: *C(=NO)C(F)(F)F] | 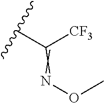 [SMILES: *C(=NOC)C(F)(F)F] | 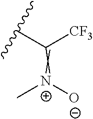 [SMILES: *C(=[N+](C)[O-])C(F)(F)F] |
| 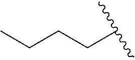 [SMILES: *CCCC] | XPF-1023 | XPF-1037 | XPF-1051 |
| 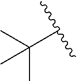 [SMILES: *C(C)(C)C] | XPF-1024 | XPF-1038 | XPF-1052 |
| 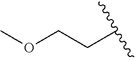 [SMILES: *CCOC] | XPF-1025 | XPF-1039 | XPF-1053 |
| 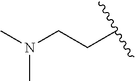 [SMILES: *CCN(C)C] | XPF-1026 | XPF-1040 | XPF-1054 |
| 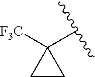 [SMILES: *C1(C(F)(F)F)CC1] | XPF-1027 | XPF-1041 | XPF-1055 |
| 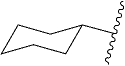 [SMILES: *C1CCCCC1] | XPF-1028 | XPF-1042 | XPF-1056 |
| 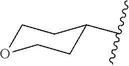 [SMILES: *C1CCOCC1] | XPF-1029 | XPF-1043 | XPF-1057 |
| 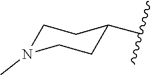 [SMILES: *C1CCN(C)CC1] | XPF-1030 | XPF-1044 | XPF-1058 |
| 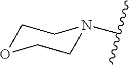 [SMILES: *N1CCOCC1] | XPF-1031 | XPF-1045 | XPF-1059 |
| 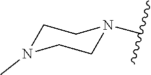 [SMILES: *N1CCN(C)CC1] | XPF-1032 | XPF-1046 | XPF-1060 |
| 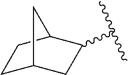 | XPF-1033 | XPF-1047 | XPF-1061 |
| 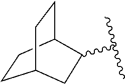 | XPF-1034 | XPF-1048 | XPF-1062 |
| 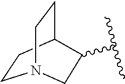 | XPF-1035 | XPF-1049 | XPF-1063 |
| 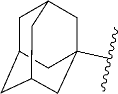 [SMILES: *C12CC3CC(CC(C3)C1)C2] | XPF-1036 | XPF-1050 | XPF-1064 |

The above table constitutes an individualized description of each of the specifically indicated compounds therein as well as their salts and solvates.

TABLE 13

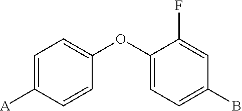

| | B | | | |
|---|---|---|---|---|
| A | 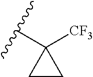 [SMILES: *C1(C(F)(F)F)CC1] | 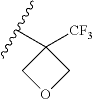 [SMILES: *C1(C(F)(F)F)COC1] | 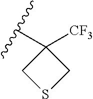 [SMILES: *C1(C(F)(F)F)CSC1] | 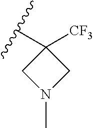 [SMILES: *C1(C(F)(F)F)CN(C)C1] |
| 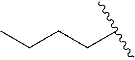 [SMILES: *CCCC] | XPF-1065 | XPF-1079 | XPF-1093 | XPF-1107 |
| 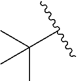 [SMILES: *C(C)(C)C] | XPF-1066 | XPF-1080 | XPF-1094 | XPF-1108 |
| 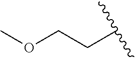 [SMILES: *CCOC] | XPF-1067 | XPF-1081 | XPF-1095 | XPF-1109 |
| 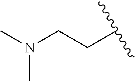 [SMILES: *CCN(C)C] | XPF-1068 | XPF-1082 | XPF-1096 | XPF-1110 |
| 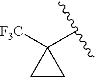 [SMILES: *C1(C(F)(F)F)CC1] | XPF-1069 | XPF-1083 | XPF-1097 | XPF-1111 |
| 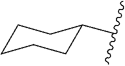 [SMILES: *C1CCCCC1] | XPF-1070 | XPF-1084 | XPF-1098 | XPF-1112 |
| 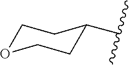 [SMILES: *C1CCOCC1] | XPF-1071 | XPF-1085 | XPF-1099 | XPF-1113 |
| 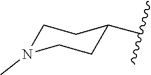 [SMILES: *C1CCN(C)CC1] | XPF-1072 | XPF-1086 | XPF-1100 | XPF-1114 |
| 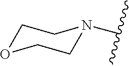 [SMILES: *N1CCOCC1] | XPF-1073 | XPF-1087 | XPF-1101 | XPF-1115 |
| 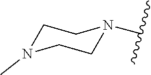 [SMILES: *N1CCN(C)CC1] | XPF-1074 | XPF-1088 | XPF-1102 | XPF-1116 |
| 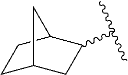 | XPF-1075 | XPF-1089 | XPF-1103 | XPF-1117 |
| 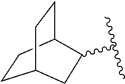 | XPF-1076 | XPF-1090 | XPF-1104 | XPF-1118 |
| 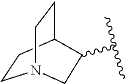 | XPF-1077 | XPF-1091 | XPF-1105 | XPF-1119 |
| 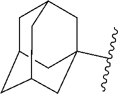 [SMILES: *C12CC3CC(CC(C3)C1)C2] | XPF-1078 | XPF-1092 | XPF-1106 | XPF-1120 |

The above table constitutes an individualized description of each of the specifically indicated compounds therein as well as their salts and solvates.

TABLE 14

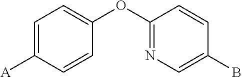

| A\B | 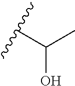 [SMILES: *C(C)O] | 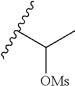 [SMILES: *C(C)OS(C)(=O)=O] | 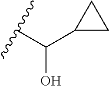 [SMILES: *C(O)C1CC1] | 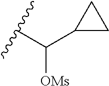 [SMILES: *C(OS(C)(=O)=O)C1CC1] | 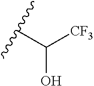 [SMILES: *C(O)C(F)(F)F] | 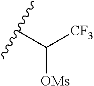 [SMILES: *C(OS(C)(=O)=O)C(F)(F)F] |
|---|---|---|---|---|---|---|
| 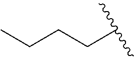 [SMILES: *CCCC] | XPF-1121 | XPF-1135 | XPF-1149 | XPF-1163 | XPF-1177 | XPF-1191 |
| 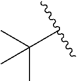 [SMILES: *C(C)(C)C] | XPF-1122 | XPF-1136 | XPF-1150 | XPF-1164 | XPF-1178 | XPF-1192 |
| 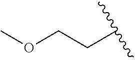 [SMILES: *CCOC] | XPF-1123 | XPF-1137 | XPF-1151 | XPF-1165 | XPF-1179 | XPF-1193 |
| 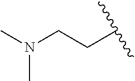 [SMILES: *CCN(C)C] | XPF-1124 | XPF-1138 | XPF-1152 | XPF-1166 | XPF-1180 | XPF-1194 |
| 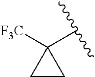 [SMILES: *C1(C(F)(F)F)CC1] | XPF-1125 | XPF-1139 | XPF-1153 | XPF-1167 | XPF-1181 | XPF-1195 |
| 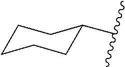 [SMILES: *C1CCCCC1] | XPF-1126 | XPF-1140 | XPF-1154 | XPF-1168 | XPF-1182 | XPF-1196 |
| 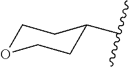 [SMILES: *C1CCOCC1] | XPF-1127 | XPF-1141 | XPF-1155 | XPF-1169 | XPF-1183 | XPF-1197 |
| 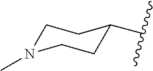 [SMILES: *C1CCN(C)CC1] | XPF-1128 | XPF-1142 | XPF-1156 | XPF-1170 | XPF-1184 | XPF-1198 |
| 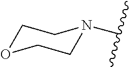 [SMILES: *N1CCOCC1] | XPF-1129 | XPF-1143 | XPF-1157 | XPF-1171 | XPF-1185 | XPF-1199 |
| 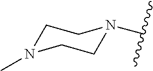 [SMILES: *N1CCN(C)CC1] | XPF-1130 | XPF-1144 | XPF-1158 | XPF-1172 | XPF-1186 | XPF-1200 |
| 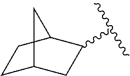 | XPF-1131 | XPF-1145 | XPF-1159 | XPF-1173 | XPF-1187 | XPF-1201 |
| 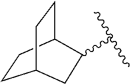 | XPF-1132 | XPF-1146 | XPF-1160 | XPF-1174 | XPF-1188 | XPF-1202 |
| 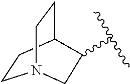 | XPF-1133 | XPF-1147 | XPF-1161 | XPF-1175 | XPF-1189 | XPF-1203 |
| 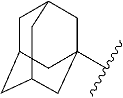 [SMILES: *C12CC3CC(CC(C3)C1)C2] | XPF-1134 | XPF-1148 | XPF-1162 | XPF-1176 | XPF-1190 | XPF-1204 |
| | A\B | 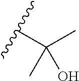 [SMILES: *C(C)(C)O] | 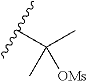 [SMILES: *C(C)(C)OS(C)(=O)=O] | 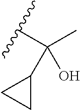 [SMILES: *C(C)(O)C1CC1] | 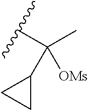 [SMILES: *C(C)(OS(C)(=O)=O)C1CC1] | 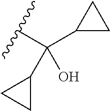 [SMILES: *C(O)(C1CC1)C1CC1] |
| | 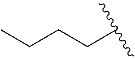 [SMILES: *CCCC] | XPF-1205 | XPF-1219 | XPF-1233 | XPF-1247 | XPF-1261 |
| | 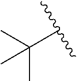 [SMILES: *C(C)(C)C] | XPF-1206 | XPF-1220 | XPF-1234 | XPF-1248 | XPF-1262 |
| | 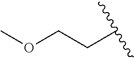 [SMILES: *CCOC] | XPF-1207 | XPF-1221 | XPF-1235 | XPF-1249 | XPF-1263 |
| | 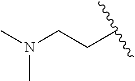 [SMILES: *CCN(C)C] | XPF-1208 | XPF-1222 | XPF-1236 | XPF-1250 | XPF-1264 |
| | 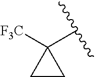 [SMILES: *C1(C(F)(F)F)CC1] | XPF-1209 | XPF-1223 | XPF-1237 | XPF-1251 | XPF-1265 |
| | 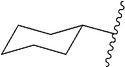 [SMILES: *C1CCCCC1] | XPF-1210 | XPF-1224 | XPF-1238 | XPF-1252 | XPF-1266 |
| | 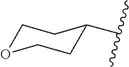 [SMILES: *C1CCOCC1] | XPF-1211 | XPF-1225 | XPF-1239 | XPF-1253 | XPF-1267 |
| | 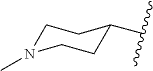 [SMILES: *C1CCN(C)CC1] | XPF-1212 | XPF-1226 | XPF-1240 | XPF-1254 | XPF-1268 |
| | 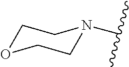 [SMILES: *N1CCOCC1] | XPF-1213 | XPF-1227 | XPF-1241 | XPF-1255 | XPF-1269 |
| | 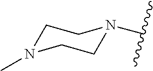 [SMILES: *N1CCN(C)CC1] | XPF-1214 | XPF-1228 | XPF-1242 | XPF-1256 | XPF-1270 |
| | 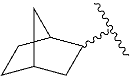 | XPF-1215 | XPF-1229 | XPF-1243 | XPF-1257 | XPF-1271 |
| | 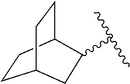 | XPF-1216 | XPF-1230 | XPF-1244 | XPF-1258 | XPF-1272 |
| | 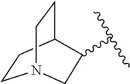 | XPF-1217 | XPF-1231 | XPF-1245 | XPF-1259 | XPF-1273 |
| | 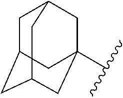 [SMILES: *C12CC3CC(CC(C3)C1)C2] | XPF-1218 | XPF-1232 | XPF-1246 | XPF-1260 | XPF-1274 |

The above table constitutes an individualized description of each of the specifically indicated compounds therein as well as their salts and solvates.

TABLE 15

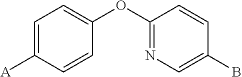

| A\B | 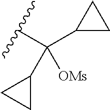 [SMILES: *C(OS(C)(=O)=O)(C1CC1)C1CC1] | 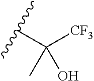 [SMILES: *C(C)(O)C(F)(F)F] | 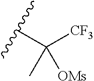 [SMILES: *C(C)(OS(C)(=O)=O)C(F)(F)F] | 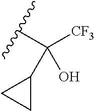 [SMILES: *C(O)(C1CC1)C(F)(F)F] | 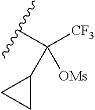 [SMILES: *C(OS(C)(=O)=O)(C1CC1)C(F)(F)F] |
|---|---|---|---|---|---|
| 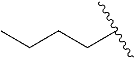 [SMILES: *CCCC] | XPF-1275 | XPF-1289 | XPF-1303 | XPF-1317 | XPF-1331 |
| 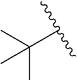 [SMILES: *C(C)(C)C] | XPF-1276 | XPF-1290 | XPF-1304 | XPF-1318 | XPF-1332 |
| 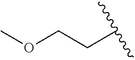 [SMILES: *CCOC] | XPF-1277 | XPF-1291 | XPF-1305 | XPF-1319 | XPF-1333 |
| 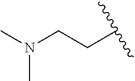 [SMILES: *CCN(C)C] | XPF-1278 | XPF-1292 | XPF-1306 | XPF-1320 | XPF-1334 |
| 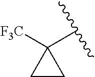 [SMILES: *C1(C(F)(F)F)CC1] | XPF-1279 | XPF-1293 | XPF-1307 | XPF-1321 | XPF-1335 |
| 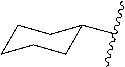 [SMILES: *C1CCCCC1] | XPF-1280 | XPF-1294 | XPF-1308 | XPF-1322 | XPF-1336 |
| 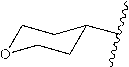 [SMILES: *C1CCOCC1] | XPF-1281 | XPF-1295 | XPF-1309 | XPF-1323 | XPF-1337 |
| 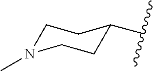 [SMILES: *C1CCN(C)CC1] | XPF-1282 | XPF-1296 | XPF-1310 | XPF-1324 | XPF-1338 |
| 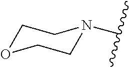 [SMILES: *N1CCOCC1] | XPF-1283 | XPF-1297 | XPF-1311 | XPF-1325 | XPF-1339 |
| 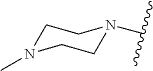 [SMILES: *N1CCN(C)CC1] | XPF-1284 | XPF-1298 | XPF-1312 | XPF-1326 | XPF-1340 |
| 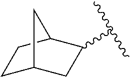 | XPF-1285 | XPF-1299 | XPF-1313 | XPF-1327 | XPF-1341 |
| 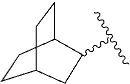 | XPF-1286 | XPF-1300 | XPF-1314 | XPF-1328 | XPF-1342 |
| 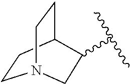 | XPF-1287 | XPF-1301 | XPF-1315 | XPF-1329 | XPF-1343 |
| 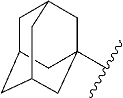 [SMILES: *C12CC3CC(CC(C3)C1)C2] | XPF-1288 | XPF-1302 | XPF-1316 | XPF-1330 | XPF-1344 |
| | | | A\B | 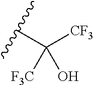 [SMILES: *C(O)(C(F)(F)F)C(F)(F)F] | 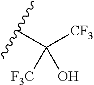 [SMILES: *C(O)(C(F)(F)F)C(F)(F)F] |
| | | | 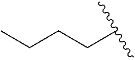 [SMILES: *CCCC] | XPF-1345 | XPF-1359 |
| | | | 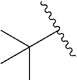 [SMILES: *C(C)(C)C] | XPF-1346 | XPF-1360 |
| | | | 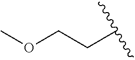 [SMILES: *CCOC] | XPF-1347 | XPF-1361 |
| | | | 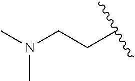 [SMILES: *CCN(C)C] | XPF-1348 | XPF-1362 |
| | | | 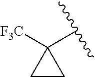 [SMILES: *C1(C(F)(F)F)CC1] | XPF-1349 | XPF-1363 |
| | | | 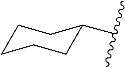 [SMILES: *C1CCCCC1] | XPF-1350 | XPF-1364 |
| | | | 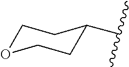 [SMILES: *C1CCOCC1] | XPF-1351 | XPF-1365 |
| | | | 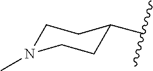 [SMILES: *C1CCN(C)CC1] | XPF-1352 | XPF-1366 |
| | | | 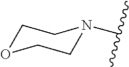 [SMILES: *N1CCOCC1] | XPF-1353 | XPF-1367 |
| | | | 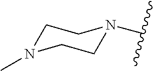 [SMILES: *N1CCN(C)CC1] | XPF-1354 | XPF-1368 |
| | | | 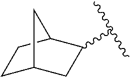 | XPF-1355 | XPF-1369 |
| | | | 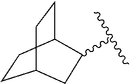 | XPF-1356 | XPF-1370 |
| | | | 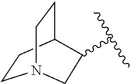 | XPF-1357 | XPF-1371 |
| | | | 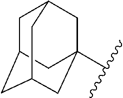 [SMILES: *C12CC3CC(CC(C3)C1)C2] | XPF-1358 | XPF-1372 |

The above table constitutes an individualized description of each of the specifically indicated compounds therein as well as their salts and solvates.

TABLE 16

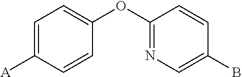

| A\B | 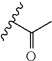 [SMILES: *C(C)=O] | 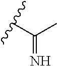 [SMILES: *C(C)=N] | 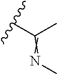 [SMILES: *C(C)=NC] | 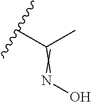 [SMILES: *C(C)=NO] | 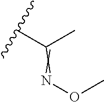 [SMILES: *C(C)=NOC] | 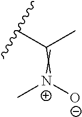 [SMILES: *C(C)=[N+](C)[O-]] | 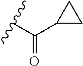 [SMILES: *C(=O)C1CC1] | 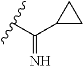 [SMILES: *C(=N)C1CC1] | 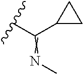 [SMILES: *C(=NC)C1CC1] | 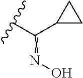 [SMILES: *C(=NO)C1CC1] | 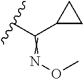 [SMILES: *C(=NOC)C1CC1] |
|---|---|---|---|---|---|---|---|---|---|---|---|
| 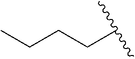 [SMILES: *CCCC] | XPF-1373 | XPF-1387 | XPF-1401 | XPF-1415 | XPF-1429 | XPF-1443 | XPF-1457 | XPF-1471 | XPF-1485 | XPF-1499 | XPF-1513 |
| 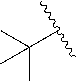 [SMILES: *C(C)(C)C] | XPF-1374 | XPF-1388 | XPF-1402 | XPF-1416 | XPF-1430 | XPF-1444 | XPF-1458 | XPF-1472 | XPF-1486 | XPF-1500 | XPF-1514 |
| 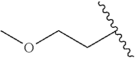 [SMILES: *CCOC] | XPF-1375 | XPF-1389 | XPF-1403 | XPF-1417 | XPF-1431 | XPF-1445 | XPF-1459 | XPF-1473 | XPF-1487 | XPF-1501 | XPF-1515 |
| 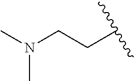 [SMILES: *CCN(C)C] | XPF-1376 | XPF-1390 | XPF-1404 | XPF-1418 | XPF-1432 | XPF-1446 | XPF-1460 | XPF-1474 | XPF-1488 | XPF-1502 | XPF-1516 |
| 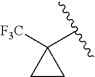 [SMILES: *C1(C(F)(F)F)CC1] | XPF-1377 | XPF-1391 | XPF-1405 | XPF-1419 | XPF-1433 | XPF-1447 | XPF-1461 | XPF-1475 | XPF-1489 | XPF-1503 | XPF-1517 |
| 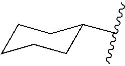 [SMILES: *C1CCCCC1] | XPF-1378 | XPF-1392 | XPF-1406 | XPF-1420 | XPF-1434 | XPF-1448 | XPF-1462 | XPF-1476 | XPF-1490 | XPF-1504 | XPF-1518 |
| 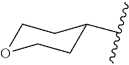 [SMILES: *C1CCOCC1] | XPF-1379 | XPF-1393 | XPF-1407 | XPF-1421 | XPF-1435 | XPF-1449 | XPF-1463 | XPF-1477 | XPF-1491 | XPF-1505 | XPF-1519 |
| 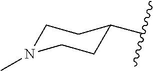 [SMILES: *C1CCN(C)CC1] | XPF-1380 | XPF-1394 | XPF-1408 | XPF-1422 | XPF-1436 | XPF-1450 | XPF-1464 | XPF-1478 | XPF-1492 | XPF-1506 | XPF-1520 |
| 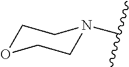 [SMILES: *N1CCOCC1] | XPF-1381 | XPF-1395 | XPF-1409 | XPF-1423 | XPF-1437 | XPF-1451 | XPF-1465 | XPF-1479 | XPF-1493 | XPF-1507 | XPF-1521 |
| 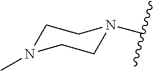 [SMILES: *N1CCN(C)CC1] | XPF-1382 | XPF-1396 | XPF-1410 | XPF-1424 | XPF-1438 | XPF-1452 | XPF-1466 | XPF-1480 | XPF-1494 | XPF-1508 | XPF-1522 |
| 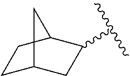 | XPF-1383 | XPF-1397 | XPF-1411 | XPF-1425 | XPF-1439 | XPF-1453 | XPF-1467 | XPF-1481 | XPF-1495 | XPF-1509 | XPF-1523 |
| 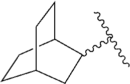 | XPF-1384 | XPF-1398 | XPF-1412 | XPF-1426 | XPF-1440 | XPF-1454 | XPF-1468 | XPF-1482 | XPF-1496 | XPF-1510 | XPF-1524 |
| 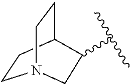 | XPF-1385 | XPF-1399 | XPF-1413 | XPF-1427 | XPF-1441 | XPF-1455 | XPF-1469 | XPF-1483 | XPF-1497 | XPF-1511 | XPF-1525 |
| 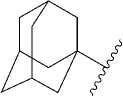 [SMILES: *C12CC3CC(CC(C3)C1)C2] | XPF-1386 | XPF-1400 | XPF-1414 | XPF-1428 | XPF-1442 | XPF-1456 | XPF-1470 | XPF-1484 | XPF-1498 | XPF-1512 | XPF-1526 |

The above table constitutes an individualized description of each of the specifically indicated compounds therein as well as their salts and solvates.

TABLE 17

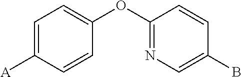

| A\B | 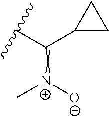 [SMILES: *C(C1CC1)=[N+](C)[O-]] | 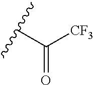 [SMILES: *C(=O)C(F)(F)F] | 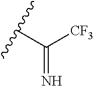 [SMILES: *C(=N)C(F)(F)F] | 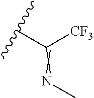 [SMILES: *C(=NC)C(F)(F)F] |
|---|---|---|---|---|
| 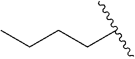 [SMILES: *CCCC] | XPF-1527 | XPF-1541 | XPF-1555 | XPF-1569 |
| 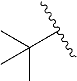 [SMILES: *C(C)(C)C] | XPF-1528 | XPF-1542 | XPF-1556 | XPF-1570 |
| 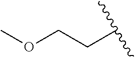 [SMILES: *CCOC] | XPF-1529 | XPF-1543 | XPF-1557 | XPF-1571 |
| 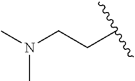 [SMILES: *CCN(C)C] | XPF-1530 | XPF-1544 | XPF-1558 | XPF-1572 |
| 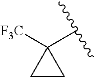 [SMILES: *C1(C(F)(F)F)CC1] | XPF-1531 | XPF-1545 | XPF-1559 | XPF-1573 |
| 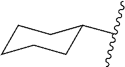 [SMILES: *C1CCCCC1] | XPF-1532 | XPF-1546 | XPF-1560 | XPF-1574 |
| 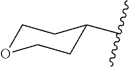 [SMILES: *C1CCOCC1] | XPF-1533 | XPF-1547 | XPF-1561 | XPF-1575 |
| 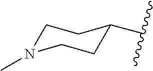 [SMILES: *C1CCN(C)CC1] | XPF-1534 | XPF-1548 | XPF-1562 | XPF-1576 |
| 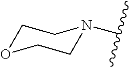 [SMILES: *N1CCOCC1] | XPF-1535 | XPF-1549 | XPF-1563 | XPF-1577 |
| 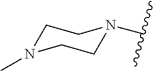 [SMILES: *N1CCN(C)CC1] | XPF-1536 | XPF-1550 | XPF-1564 | XPF-1578 |
| 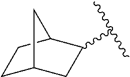 | XPF-1537 | XPF-1551 | XPF-1565 | XPF-1579 |
| 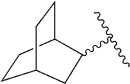 | XPF-1538 | XPF-1552 | XPF-1566 | XPF-1580 |
| 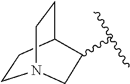 | XPF-1539 | XPF-1553 | XPF-1567 | XPF-1581 |
| 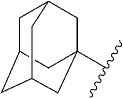 [SMILES: *C12CC3CC(CC(C3)C1)C2] | XPF-1540 | XPF-1554 | XPF-1568 | XPF-1582 |
| | A\B | 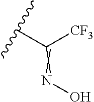 [SMILES: *C(=NO)C(F)(F)F] | 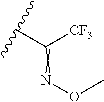 [SMILES: *C(=NOC)C(F)(F)F] | 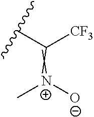 [SMILES: *C(=[N+](C)[O-])C(F)(F)F] |
| | 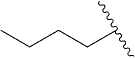 [SMILES: *CCCC] | XPF-1583 | XPF-1597 | XPF-1611 |
| | 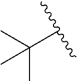 [SMILES: *C(C)(C)C] | XPF-1584 | XPF-1598 | XPF-1612 |
| | 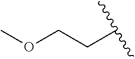 [SMILES: *CCOC] | XPF-1585 | XPF-1599 | XPF-1613 |
| | 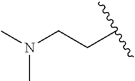 [SMILES: *CCN(C)C] | XPF-1586 | XPF-1600 | XPF-1614 |
| | 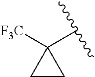 [SMILES: *C1(C(F)(F)F)CC1] | XPF-1587 | XPF-1601 | XPF-1615 |
| | 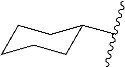 [SMILES: *C1CCCCC1] | XPF-1588 | XPF-1602 | XPF-1616 |
| | 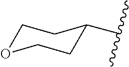 [SMILES: *C1CCOCC1] | XPF-1589 | XPF-1603 | XPF-1617 |
| | 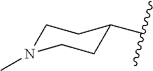 [SMILES: *C1CCN(C)CC1] | XPF-1590 | XPF-1604 | XPF-1618 |
| | 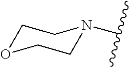 [SMILES: *N1CCOCC1] | XPF-1591 | XPF-1605 | XPF-1619 |
| | 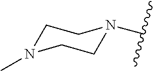 [SMILES: *N1CCN(C)CC1] | XPF-1592 | XPF-1606 | XPF-1620 |
| | 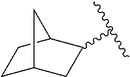 | XPF-1593 | XPF-1607 | XPF-1621 |
| | 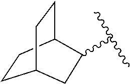 | XPF-1594 | XPF-1608 | XPF-1622 |
| | 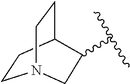 | XPF-1595 | XPF-1609 | XPF-1623 |
| | 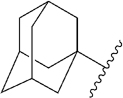 [SMILES: *C12CC3CC(CC(C3)C1)C2] | XPF-1596 | XPF-1610 | XPF-1624 |

The above table constitutes an individualized description of each of the specifically indicated compounds therein as well as their salts and solvates.

TABLE 18

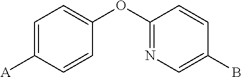

| A\B | 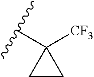 [SMILES: *C1(C(F)(F)F)CC1] | 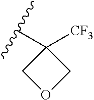 [SMILES: *C1(C(F)(F)F)COC1] | 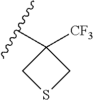 [SMILES: *C1(C(F)(F)F)CSC1] | 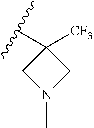 [SMILES: *C1(C(F)(F)F)CN(C)C1] |
|---|---|---|---|---|
| 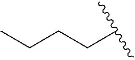 [SMILES: *CCCC] | XPF-1625 | XPF-1639 | XPF-1653 | XPF-1667 |
| 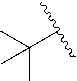 [SMILES: *C(C)(C)C] | XPF-1626 | XPF-1640 | XPF-1654 | XPF-1668 |
| 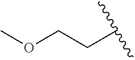 [SMILES: *CCOC] | XPF-1627 | XPF-1641 | XPF-1655 | XPF-1669 |
| 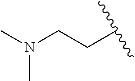 [SMILES: *CCN(C)C] | XPF-1628 | XPF-1642 | XPF-1656 | XPF-1670 |
| 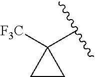 [SMILES: *C1(C(F)(F)F)CC1] | XPF-1629 | XPF-1643 | XPF-1657 | XPF-1671 |
| 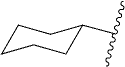 [SMILES: *C1CCCCC1] | XPF-1630 | XPF-1644 | XPF-1658 | XPF-1672 |
| 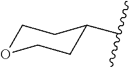 [SMILES: *C1CCOCC1] | XPF-1631 | XPF-1645 | XPF-1659 | XPF-1673 |
| 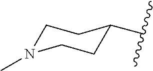 [SMILES: *C1CCN(C)CC1] | XPF-1632 | XPF-1646 | XPF-1660 | XPF-1674 |
| 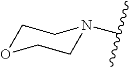 [SMILES: *N1CCOCC1] | XPF-1633 | XPF-1647 | XPF-1661 | XPF-1675 |
| 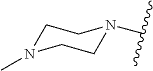 [SMILES: *N1CCN(C)CC1] | XPF-1634 | XPF-1648 | XPF-1662 | XPF-1676 |
| 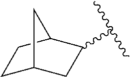 | XPF-1635 | XPF-1649 | XPF-1663 | XPF-1677 |
| 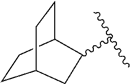 | XPF-1636 | XPF-1650 | XPF-1664 | XPF-1678 |
| 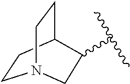 | XPF-1637 | XPF-1651 | XPF-1665 | XPF-1679 |
| 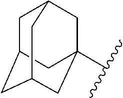 [SMILES: *C12CC3CC(CC(C3)C1)C2] | XPF-1638 | XPF-1652 | XPF-1666 | XPF-1680 |

The above table constitutes an individualized description of each of the specifically indicated compounds therein as well as their salts and solvates.

TABLE 19

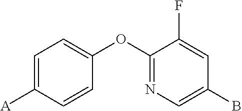

| A\B | 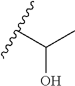 [SMILES: *C(C)O] | 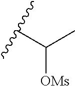 [SMILES: *C(C)OS(C)(=O)=O] | 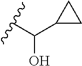 [SMILES: *C(O)C1CC1] | 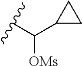 [SMILES: *C(OS(C)(=O)=O)C1CC1] | 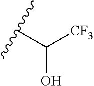 [SMILES: *C(O)C(F)(F)F] | 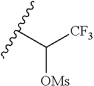 [SMILES: *C(OS(C)(=O)=O)C(F)(F)F] | 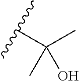 [SMILES: *C(C)(C)O] | 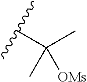 [SMILES: *C(C)(C)OS(C)(=O)=O] | 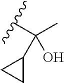 [SMILES: *C(C)(O)C1CC1] | 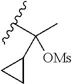 [SMILES: *C(C)(OS(C)(=O)=O)C1CC1] | 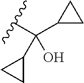 [SMILES: *C(O)(C1CC1)C1CC1] |
|---|---|---|---|---|---|---|---|---|---|---|---|
| 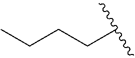 [SMILES: *CCCC] | XPF-1681 | XPF-1695 | XPF-1709 | XPF-1723 | XPF-1737 | XPF-1751 | XPF-1765 | XPF-1779 | XPF-1793 | XPF-1807 | XPF-1821 |
| 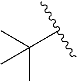 [SMILES: *C(C)(C)C] | XPF-1682 | XPF-1696 | XPF-1710 | XPF-1724 | XPF-1738 | XPF-1752 | XPF-1766 | XPF-1780 | XPF-1794 | XPF-1808 | XPF-1822 |
| 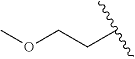 [SMILES: *CCOC] | XPF-1683 | XPF-1697 | XPF-1711 | XPF-1725 | XPF-1739 | XPF-1753 | XPF-1767 | XPF-1781 | XPF-1795 | XPF-1809 | XPF-1823 |
| 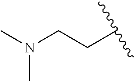 [SMILES: *CCN(C)C] | XPF-1684 | XPF-1698 | XPF-1712 | XPF-1726 | XPF-1740 | XPF-1754 | XPF-1768 | XPF-1782 | XPF-1796 | XPF-1810 | XPF-1824 |
| 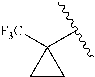 [SMILES: *C1(C(F)(F)F)CC1] | XPF-1685 | XPF-1699 | XPF-1713 | XPF-1727 | XPF-1741 | XPF-1755 | XPF-1769 | XPF-1783 | XPF-1797 | XPF-1811 | XPF-1825 |
| 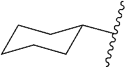 [SMILES: *C1CCCCC1] | XPF-1686 | XPF-1700 | XPF-1714 | XPF-1728 | XPF-1742 | XPF-1756 | XPF-1770 | XPF-1784 | XPF-1798 | XPF-1812 | XPF-1826 |
| 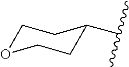 [SMILES: *C1CCOCC1] | XPF-1687 | XPF-1701 | XPF-1715 | XPF-1729 | XPF-1743 | XPF-1757 | XPF-1771 | XPF-1785 | XPF-1799 | XPF-1813 | XPF-1827 |
| 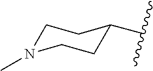 [SMILES: *C1CCN(C)CC1] | XPF-1688 | XPF-1702 | XPF-1716 | XPF-1730 | XPF-1744 | XPF-1758 | XPF-1772 | XPF-1786 | XPF-1800 | XPF-1814 | XPF-1828 |
| 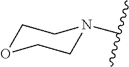 [SMILES: *N1CCOCC1] | XPF-1689 | XPF-1703 | XPF-1717 | XPF-1731 | XPF-1745 | XPF-1759 | XPF-1773 | XPF-1787 | XPF-1801 | XPF-1815 | XPF-1829 |
| 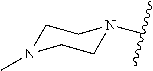 [SMILES: *N1CCN(C)CC1] | XPF-1690 | XPF-1704 | XPF-1718 | XPF-1732 | XPF-1746 | XPF-1760 | XPF-1774 | XPF-1788 | XPF-1802 | XPF-1816 | XPF-1830 |
| 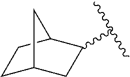 | XPF-1691 | XPF-1705 | XPF-1719 | XPF-1733 | XPF-1747 | XPF-1761 | XPF-1775 | XPF-1789 | XPF-1803 | XPF-1817 | XPF-1831 |
| 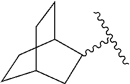 | XPF-1692 | XPF-1706 | XPF-1720 | XPF-1734 | XPF-1748 | XPF-1762 | XPF-1776 | XPF-1790 | XPF-1804 | XPF-1818 | XPF-1832 |
| 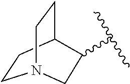 | XPF-1693 | XPF-1707 | XPF-1721 | XPF-1735 | XPF-1749 | XPF-1763 | XPF-1777 | XPF-1791 | XPF-1805 | XPF-1819 | XPF-1833 |
| 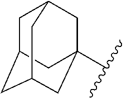 [SMILES: *C12CC3CC(CC(C3)C1)C2] | XPF-1694 | XPF-1708 | XPF-1722 | XPF-1736 | XPF-1750 | XPF-1764 | XPF-1778 | XPF-1792 | XPF-1806 | XPF-1820 | XPF-1834 |

The above table constitutes an individualized description of each of the specifically indicated compounds therein as well as their salts and solvates.

TABLE 20

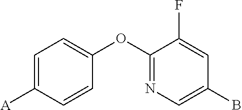

| A\B | 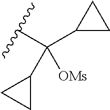 [SMILES: *C(OS(C)(=O)=O)(C1CC1)C1CC1] | 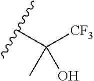 [SMILES: *C(C)(O)C(F)(F)F] | 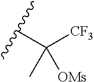 [SMILES: *C(C)(OS(C)(=O)=O)C(F)(F)F] | 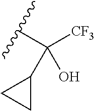 [SMILES: *C(O)(C1CC1)C(F)(F)F] |
|---|---|---|---|---|
| 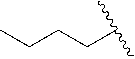 [SMILES: *CCCC] | XPF-1835 | XPF-1849 | XPF-1863 | XPF-1877 |
| 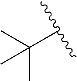 [SMILES: *C(C)(C)C] | XPF-1836 | XPF-1850 | XPF-1864 | XPF-1878 |
| 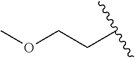 [SMILES: *CCOC] | XPF-1837 | XPF-1851 | XPF-1865 | XPF-1879 |
| 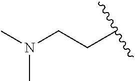 [SMILES: *CCN(C)C] | XPF-1838 | XPF-1852 | XPF-1866 | XPF-1880 |
| 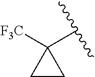 [SMILES: *C1(C(F)(F)F)CC1] | XPF-1839 | XPF-1853 | XPF-1867 | XPF-1881 |
| 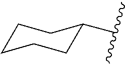 [SMILES: *C1CCCCC1] | XPF-1840 | XPF-1854 | XPF-1868 | XPF-1882 |
| 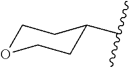 [SMILES: *C1CCOCC1] | XPF-1841 | XPF-1855 | XPF-1869 | XPF-1883 |
| 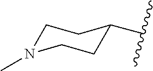 [SMILES: *C1CCN(C)CC1] | XPF-1842 | XPF-1856 | XPF-1870 | XPF-1884 |
| 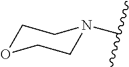 [SMILES: *N1CCOCC1] | XPF-1843 | XPF-1857 | XPF-1871 | XPF-1885 |
| 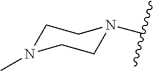 [SMILES: *N1CCN(C)CC1] | XPF-1844 | XPF-1858 | XPF-1872 | XPF-1886 |
| 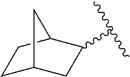 | XPF-1845 | XPF-1859 | XPF-1873 | XPF-1887 |
| 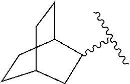 | XPF-1846 | XPF-1860 | XPF-1874 | XPF-1888 |
| 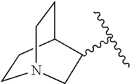 | XPF-1847 | XPF-1861 | XPF-1875 | XPF-1889 |
| 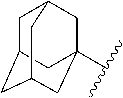 [SMILES: *C12CC3CC(CC(C3)C1)C2] | XPF-1848 | XPF-1862 | XPF-1876 | XPF-1890 |
| | A\B | 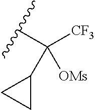 [SMILES: *C(OS(C)(=O)=O)(C1CC1)C(F)(F)F] | 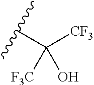 [SMILES: *C(O)(C(F)(F)F)C(F)(F)F] | 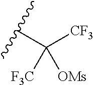 [SMILES: *C(OS(C)(=O)=O)(C(F)(F)F)C(F)(F)F] |
| | 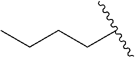 [SMILES: *CCCC] | XPF-1891 | XPF-1905 | XPF-1919 |
| | 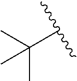 [SMILES: *C(C)(C)C] | XPF-1892 | XPF-1906 | XPF-1920 |
| | 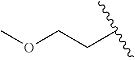 [SMILES: *CCOC] | XPF-1893 | XPF-1907 | XPF-1921 |
| | 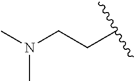 [SMILES: *CCN(C)C] | XPF-1894 | XPF-1908 | XPF-1922 |
| | 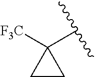 [SMILES: *C1(C(F)(F)F)CC1] | XPF-1895 | XPF-1909 | XPF-1923 |
| | 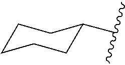 [SMILES: *C1CCCCC1] | XPF-1896 | XPF-1910 | XPF-1924 |
| | 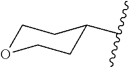 [SMILES: *C1CCOCC1] | XPF-1897 | XPF-1911 | XPF-1925 |
| | 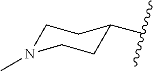 [SMILES: *C1CCN(C)CC1] | XPF-1898 | XPF-1912 | XPF-1926 |
| | 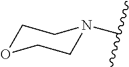 [SMILES: *N1CCOCC1] | XPF-1899 | XPF-1913 | XPF-1927 |
| | 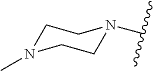 [SMILES: *N1CCN(C)CC1] | XPF-1900 | XPF-1914 | XPF-1928 |
| | 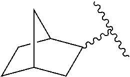 | XPF-1901 | XPF-1915 | XPF-1929 |
| | 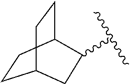 | XPF-1902 | XPF-1916 | XPF-1930 |
| | 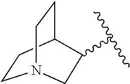 | XPF-1903 | XPF-1917 | XPF-1931 |
| | 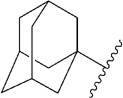 [SMILES: *C12CC3CC(CC(C3)C1)C2] | XPF-1904 | XPF-1918 | XPF-1932 |

The above table constitutes an individualized description of each of the specifically indicated compounds therein as well as their salts and solvates.

TABLE 21

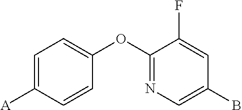

| A\B | 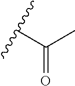 [SMILES: *C(C)=O] | 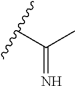 [SMILES: *C(C)=N] | 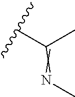 [SMILES: *C(C)=NC] | 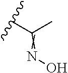 [SMILES: *C(C)=NO] | 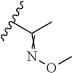 [SMILES: *C(C)=NOC] | 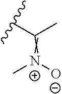 [SMILES: *C(C)=[N+](C)[O-]] | 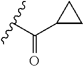 [SMILES: *C(=O)C1CC1] | 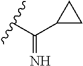 [SMILES: *C(=N)C1CC1] | 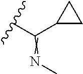 [SMILES: *C(=NC)C1CC1] | 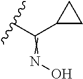 [SMILES: *C(=NO)C1CC1] | 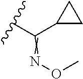 [SMILES: *C(=NOC)C1CC1] |
|---|---|---|---|---|---|---|---|---|---|---|---|
| 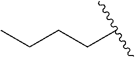 [SMILES: *CCCC] | XPF-1933 | XPF-1947 | XPF-1961 | XPF-1975 | XPF-1989 | XPF-2003 | XPF-2017 | XPF-2031 | XPF-2045 | XPF-2059 | XPF-2073 |
| 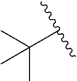 [SMILES: *C(C)(C)C] | XPF-1934 | XPF-1948 | XPF-1962 | XPF-1976 | XPF-1990 | XPF-2004 | XPF-2018 | XPF-2032 | XPF-2046 | XPF-2060 | XPF-2074 |
| 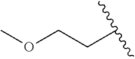 [SMILES: *CCOC] | XPF-1935 | XPF-1949 | XPF-1963 | XPF-1977 | XPF-1991 | XPF-2005 | XPF-2019 | XPF-2033 | XPF-2047 | XPF-2061 | XPF-2075 |
| 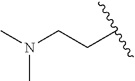 [SMILES: *CCN(C)C] | XPF-1936 | XPF-1950 | XPF-1964 | XPF-1978 | XPF-1992 | XPF-2006 | XPF-2020 | XPF-2034 | XPF-2048 | XPF-2062 | XPF-2076 |
| 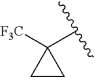 [SMILES: *C1(C(F)(F)F)CC1] | XPF-1937 | XPF-1951 | XPF-1965 | XPF-1979 | XPF-1993 | XPF-2007 | XPF-2021 | XPF-2035 | XPF-2049 | XPF-2063 | XPF-2077 |
| 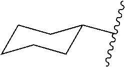 [SMILES: *C1CCCCC1] | XPF-1938 | XPF-1952 | XPF-1966 | XPF-1980 | XPF-1994 | XPF-2008 | XPF-2022 | XPF-2036 | XPF-2050 | XPF-2064 | XPF-2078 |
| 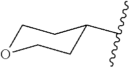 [SMILES: *C1CCOCC1] | XPF-1939 | XPF-1953 | XPF-1967 | XPF-1981 | XPF-1995 | XPF-2009 | XPF-2023 | XPF-2037 | XPF-2051 | XPF-2065 | XPF-2079 |
| 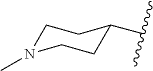 [SMILES: *C1CCN(C)CC1] | XPF-1940 | XPF-1954 | XPF-1968 | XPF-1982 | XPF-1996 | XPF-2010 | XPF-2024 | XPF-2038 | XPF-2052 | XPF-2066 | XPF-2080 |
| 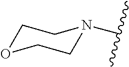 [SMILES: *N1CCOCC1] | XPF-1941 | XPF-1955 | XPF-1969 | XPF-1983 | XPF-1997 | XPF-2011 | XPF-2025 | XPF-2039 | XPF-2053 | XPF-2067 | XPF-2081 |
| 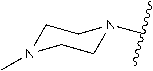 [SMILES: *N1CCN(C)CC1] | XPF-1942 | XPF-1956 | XPF-1970 | XPF-1984 | XPF-1998 | XPF-2012 | XPF-2026 | XPF-2040 | XPF-2054 | XPF-2068 | XPF-2082 |
| 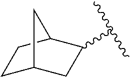 | XPF-1943 | XPF-1957 | XPF-1971 | XPF-1985 | XPF-1999 | XPF-2013 | XPF-2027 | XPF-2041 | XPF-2055 | XPF-2069 | XPF-2083 |
| 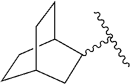 | XPF-1944 | XPF-1958 | XPF-1972 | XPF-1986 | XPF-2000 | XPF-2014 | XPF-2028 | XPF-2042 | XPF-2056 | XPF-2070 | XPF-2084 |
| 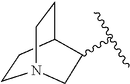 | XPF-1945 | XPF-1959 | XPF-1973 | XPF-1987 | XPF-2001 | XPF-2015 | XPF-2029 | XPF-2043 | XPF-2057 | XPF-2071 | XPF-2085 |
| 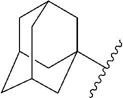 [SMILES: *C12CC3CC(CC(C3)C1)C2] | XPF-1946 | XPF-1960 | XPF-1974 | XPF-1988 | XPF-2002 | XPF-2016 | XPF-2030 | XPF-2044 | XPF-2058 | XPF-2072 | XPF-2086 |

The above table constitutes an individualized description of each of the specifically indicated compounds therein as well as their salts and solvates.

TABLE 22

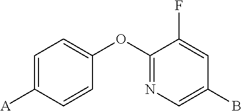

| A\B | 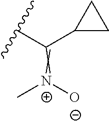 [SMILES: *C(C1CC1)=[N+](C)[O-]] | 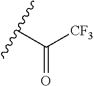 [SMILES: *C(=O)C(F)(F)F] | 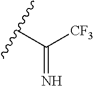 [SMILES: *C(=N)C(F)(F)F] | 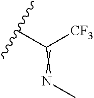 [SMILES: *C(=NC)C(F)(F)F] |
|---|---|---|---|---|
| 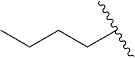 [SMILES: *CCCC] | XPF-2087 | XPF-2101 | XPF-2115 | XPF-2129 |
| 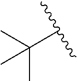 [SMILES: *C(C)(C)C] | XPF-2088 | XPF-2102 | XPF-2116 | XPF-2130 |
| 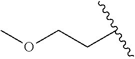 [SMILES: *CCOC] | XPF-2089 | XPF-2103 | XPF-2117 | XPF-2131 |
| 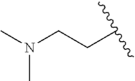 [SMILES: *CCN(C)C] | XPF-2090 | XPF-2104 | XPF-2118 | XPF-2132 |
| 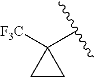 [SMILES: *C1(C(F)(F)F)CC1] | XPF-2091 | XPF-2105 | XPF-2119 | XPF-2133 |
| 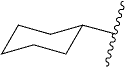 [SMILES: *C1CCCCC1] | XPF-2092 | XPF-2106 | XPF-2120 | XPF-2134 |
| 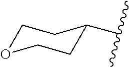 [SMILES: *C1CCOCC1] | XPF-2093 | XPF-2107 | XPF-2121 | XPF-2135 |
| 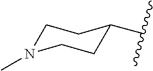 [SMILES: *C1CCN(C)CC1] | XPF-2094 | XPF-2108 | XPF-2122 | XPF-2136 |
| 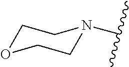 [SMILES: *N1CCOCC1] | XPF-2095 | XPF-2109 | XPF-2123 | XPF-2137 |
| 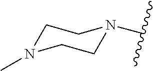 [SMILES: *N1CCN(C)CC1] | XPF-2096 | XPF-2110 | XPF-2124 | XPF-2138 |
| 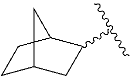 | XPF-2097 | XPF-2111 | XPF-2125 | XPF-2139 |
| 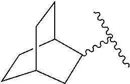 | XPF-2098 | XPF-2112 | XPF-2126 | XPF-2140 |
| 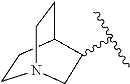 | XPF-2099 | XPF-2113 | XPF-2127 | XPF-2141 |
| 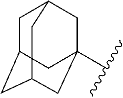 [SMILES: *C12CC3CC(CC(C3)C1)C2] | XPF-2100 | XPF-2114 | XPF-2128 | XPF-2142 |
| | A\B | 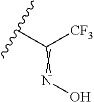 [SMILES: *C(=NO)C(F)(F)F] | 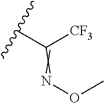 [SMILES: *C(=NOC)C(F)(F)F] | 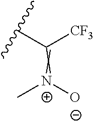 [SMILES: *C(=[N+](C)[O-])C(F)(F)F] |
| | 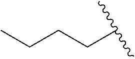 [SMILES: *CCCC] | XPF-2143 | XPF-2157 | XPF-2171 |
| | 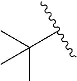 [SMILES: *C(C)(C)C] | XPF-2144 | XPF-2158 | XPF-2172 |
| | 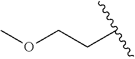 [SMILES: *CCOC] | XPF-2145 | XPF-2159 | XPF-2173 |
| | 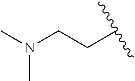 [SMILES: *CCN(C)C] | XPF-2146 | XPF-2160 | XPF-2174 |
| | 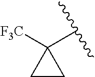 [SMILES: *C1(C(F)(F)F)CC1] | XPF-2147 | XPF-2161 | XPF-2175 |
| | 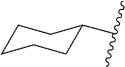 [SMILES: *C1CCCCC1] | XPF-2148 | XPF-2162 | XPF-2176 |
| | 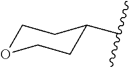 [SMILES: *C1CCOCC1] | XPF-2149 | XPF-2163 | XPF-2177 |
| | 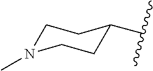 [SMILES: *C1CCN(C)CC1] | XPF-2150 | XPF-2164 | XPF-2178 |
| | 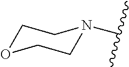 [SMILES: *N1CCOCC1] | XPF-2151 | XPF-2165 | XPF-2179 |
| | 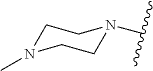 [SMILES: *N1CCN(C)CC1] | XPF-2152 | XPF-2166 | XPF-2180 |
| | 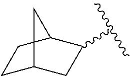 | XPF-2153 | XPF-2167 | XPF-2181 |
| | 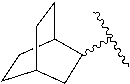 | XPF-2154 | XPF-2168 | XPF-2182 |
| | 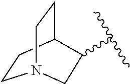 | XPF-2155 | XPF-2169 | XPF-2183 |
| | 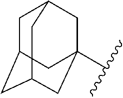 [SMILES: *C12CC3CC(CC(C3)C1)C2] | XPF-2156 | XPF-2170 | XPF-2184 |

The above table constitutes an individualized description of each of the specifically indicated compounds therein as well as their salts and solvates.

TABLE 23

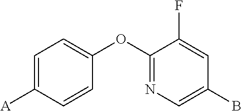

| A\B | 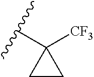 [SMILES: *C1(C(F)(F)F)CC1] | 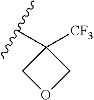 [SMILES: *C1(C(F)(F)F)COC1] | 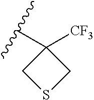 [SMILES: *C1(C(F)(F)F)CSC1] | 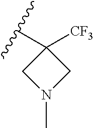 [SMILES: *C1(C(F)(F)F)CN(C)C1] |
|---|---|---|---|---|
| 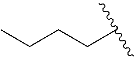 [SMILES: *CCCC] | XPF-2185 | XPF-2199 | XPF-2213 | XPF-2227 |
| 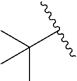 [SMILES: *C(C)(C)C] | XPF-2186 | XPF-2200 | XPF-2214 | XPF-2228 |
| 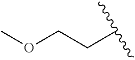 [SMILES: *CCOC] | XPF-2187 | XPF-2201 | XPF-2215 | XPF-2229 |
| 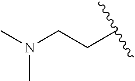 [SMILES: *CCN(C)C] | XPF-2188 | XPF-2202 | XPF-2216 | XPF-2230 |
| 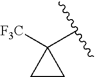 [SMILES: *C1(C(F)(F)F)CC1] | XPF-2189 | XPF-2203 | XPF-2217 | XPF-2231 |
| 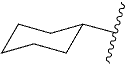 [SMILES: *C1CCCCC1] | XPF-2190 | XPF-2204 | XPF-2218 | XPF-2232 |
| 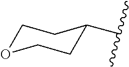 [SMILES: *C1CCOCC1] | XPF-2191 | XPF-2205 | XPF-2219 | XPF-2233 |
| 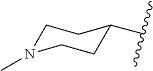 [SMILES: *C1CCN(C)CC1] | XPF-2192 | XPF-2206 | XPF-2220 | XPF-2234 |
| 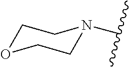 [SMILES: *N1CCOCC1] | XPF-2193 | XPF-2207 | XPF-2221 | XPF-2235 |
| 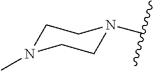 [SMILES: *N1CCN(C)CC1] | XPF-2194 | XPF-2208 | XPF-2222 | XPF-2236 |
| 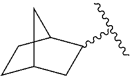 | XPF-2195 | XPF-2209 | XPF-2223 | XPF-2237 |
| 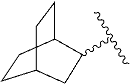 | XPF-2196 | XPF-2210 | XPF-2224 | XPF-2238 |
| 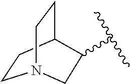 | XPF-2197 | XPF-2211 | XPF-2225 | XPF-2239 |
| 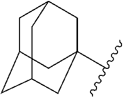 [SMILES: *C12CC3CC(CC(C3)C1)C2] | XPF-2198 | XPF-2212 | XPF-2226 | XPF-2240 |

The above table constitutes an individualized description of each of the specifically indicated compounds therein as well as their salts and solvates.

TABLE 24

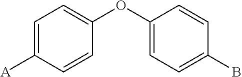

| A\B | 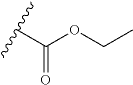 [SMILES: *C(=O)OCC] | 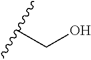 [SMILES: *CO] | 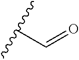 [SMILES: *C=O] |
|---|---|---|---|
| 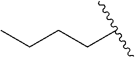 [SMILES: *CCCC] | XPF-I-0003 | XPF-I-0014 | XPF-I-0025 |
| 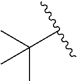 [SMILES: *C(C)(C)C] | XPF-I-0001 | XPF-I-0015 | XPF-I-0026 |
| 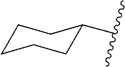 [SMILES: *C1CCCCC1] | XPF-I-0002 | XPF-I-0012 | XPF-I-0023 |
| 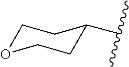 [SMILES: *C1CCOCC1] | XPF-I-0005 | XPF-I-0020 | XPF-I-0031 |
| 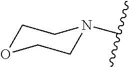 [SMILES: *N1CCOCC1] | XPF-I-0041 | XPF-I-0043 | |
| 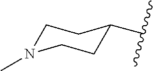 [SMILES: *C1CCN(C)CC1] | XPF-I-0006 | XPF-I-0021 | XPF-I-0032 |
| 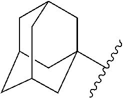 [SMILES: *C12CC3CC(CC(C3)C1)C2] | XPF-I-0004 | XPF-I-0016 | XPF-I-0027 |

The above table constitutes an individualized description of each of the specifically indicated compounds therein as well as their salts and solvates, and intermediates as well as their salts and solvates used for the synthesis of the specifically indicated compounds. Intermediates as such as well as their salts and solvates are also part of the invention, also in the frame of the process of generating the final compounds.

TABLE 25

| | | | |
|---|---|---|---|
| A\B | 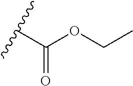 [SMILES: *C(=O)OCC] | 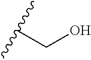 [SMILES: *CO] | 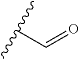 [SMILES: *C=O] |

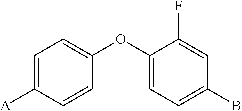

| | | | |
|---|---|---|---|
| 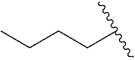 [SMILES: *CCCC] | | | |
| 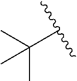 [SMILES: *C(C)(C)C] | | | |
| 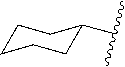 [SMILES: *C1CCCCC1] | | | |
| 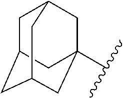 [SMILES: *C12CC3CC(CC(C3)C1)C2] | XPF-I-0007 | XPF-I-0022 | XPF-I-0033 |
| 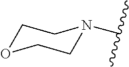 [SMILES: *N1CCOCC1] | | | |

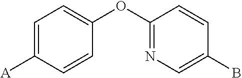

| | | | |
|---|---|---|---|
| 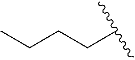 [SMILES: *CCCC] | XPF-I-0010 | XPF-I-0017 | XPF-I-0028 |
| 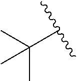 [SMILES: *C(C)(C)C] | XPF-I-0009 | XPF-I-0018 | XPF-I-0029 |
| 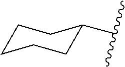 [SMILES: *C1CCCCC1] | XPF-I-0008 | XPF-I-0013 | XPF-I-0024 |
| 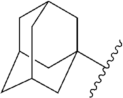 [SMILES: *C12CC3CC(CC(C3)C1)C2] | XPF-I-0011 | XPF-I-0019 | XPF-I-0030 |
| 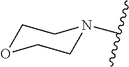 [SMILES: *N1CCOCC1] | XPF-I-0042 | XPF-I-0058 | |

The above table constitutes an individualized description of each of the specifically indicated compounds therein as well as their salts and solvates, and intermediates as well as their salts and solvates used for the synthesis of the specifically indicated compounds. Intermediates as such as well as their salts and solvates are also part of the invention, also in the frame of the process of generating the final compounds.

TABLE 26

| | | | | | |
|---|---|---|---|---|---|
| A\B | 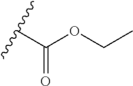 [SMILES: *C(=O)OCC] | 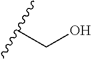 [SMILES: *CO] | 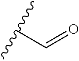 [SMILES: *C=O] | 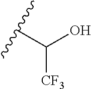 [SMILES: *C(O)C(F)(F)F] | 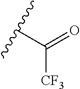 [SMILES: *C(=O)C(F)(F)F] |

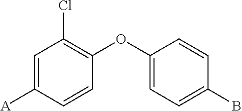

| | | | | | |
|---|---|---|---|---|---|
| 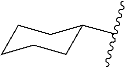 [SMILES: *C1CCCCC1] | XPF-I-0037 | XPF-I-0044 | XPF-I-0048 | XPF-2241 | XPF-2255 |
| 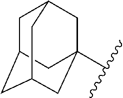 [SMILES: *C12CC3CC(CC(C3)C1)C2] | XPF-I-0038 | XPF-I-0045 | XPF-I-0049 | XPA-2242 | XPF-2245 |

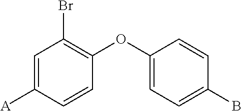

| | | | | | |
|---|---|---|---|---|---|
| 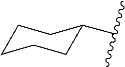 [SMILES: *C1CCCCC1] | XPF-I-0039 | XPF-I-0046 | XPF-I-0050 | XPF-2243 | XPF-2246 |
| 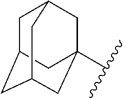 [SMILES: *C12CC3CC(CC(C3)C1)C2] | XPF-I-0040 | XPF-I-0047 | XPF-I-0051 | XPF-2244 | XPF-2247 |

The above table constitutes an individualized description of each of the specifically indicated compounds therein as well as their salts and solvates, and intermediates as well as their salts and solvates used for the synthesis of the specifically indicated compounds. Intermediates as such as well as their salts and solvates are also part of the invention, also in the frame of the process of generating the final compounds.

TABLE 27

| | | | | | | |
|---|---|---|---|---|---|---|
| A\B | 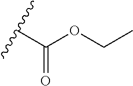 [SMILES: *C(=O)OCC] | 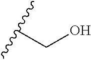 [SMILES: *CO] | 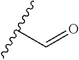 [SMILES: *C=O] | 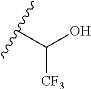 [SMILES: *C(O)C(F)(F)F] | 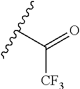 [SMILES: *C(=O)C(F)(F)F] | 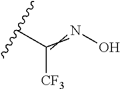 [SMILES: *C(=NO)C(F)(F)F] |

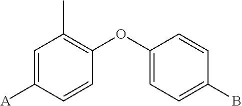

| | | | | | | |
|---|---|---|---|---|---|---|
| 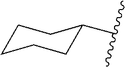 [SMILES: *C1CCCCC1] | XPF-I-0034 | XPF-I-0035 | XPF-I-0036 | XPF-2248 | XPF-2250 | XPF-2249 |
| 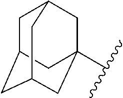 [SMILES: *C12CC3CC(CC(C3)C1)C2] | | | | XPF-2256 | XPF-2257 | XPF-2258 |

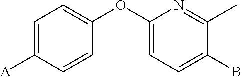

| | | | | | |
|---|---|---|---|---|---|
| 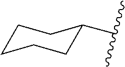 [SMILES: *C1CCCCC1] | | | | XPF-2259 | XPF-2260 |
| 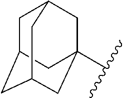 [SMILES: *C12CC3CC(CC(C3)C1)C2] | XPF-I-0055 | XPF-I-0054 | XPF-I-0057 | XPF-2252 | XPF-2253 |

The above table constitutes an individualized description of each of the specifically indicated compounds therein as well as their salts and solvates, and intermediates as well as their salts and solvates used for the synthesis of the specifically indicated compounds. Intermediates as such as well as their salts and solvates are also part of the invention, also in the frame of the process of generating the final compounds.

TABLE 28

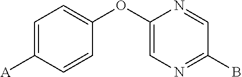

| A\B | 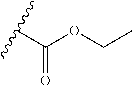 [SMILES: *C(=O)OCC] | 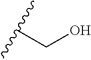 [SMILES: *CO] | 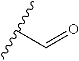 [SMILES: *C=O] | 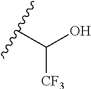 [SMILES: *C(O)C(F)(F)F] | 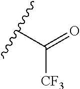 [SMILES: *C(=O)C(F)(F)F] |
|---|---|---|---|---|---|
| 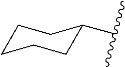 [SMILES: *C1CCCCC1] | | | | XPF-2261 | XPF-2262 |
| 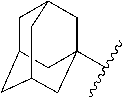 [SMILES: *C12CC3CC(CC(C3)C1)C2] | XPF-I-0052 | XPF-I-0053 | XPF-I-0056 | XPF-2251 | XPF-2254 |

The above table constitutes an individualized description of each of the specifically indicated compounds therein as well as their salts and solvates, and intermediates as well as their salts and solvates used for the synthesis of the specifically indicated compounds. Intermediates as such as well as their salts and solvates are also part of the invention, also in the frame of the process of generating the final compounds. Also included are isomers, e.g. enantiomers or diastereomers or mixtures of isomers, salts, particularly pharmaceutically acceptable salts, and solvates of the compounds listed above.

### Further Definitions

The term “C₁-C₁₂ alkyl” comprises all isomers of the corresponding saturated aliphatic hydrocarbon groups containing one to twelve carbon atoms; this includes methyl, ethyl, n-propyl, iso-propyl, n-butyl, sec-butyl, iso-butyl, tert-butyl, n-pentyl, sec-pentyl, 3-pentyl, 2-methylbutyl, iso-pentyl, 2-methylbut-2-yl, 3-methylbut-2-yl, all hexyl-isomers, all heptyl-isomers, all octyl-isomers, all nonyl-isomers, all decyl-isomers, all undecyl-isomers and all dodecyl-isomers.

The term “C₂-C₁₂ alkenyl” comprises all isomers of the corresponding unsaturated olefinic hydrocarbon groups containing two to twelve carbon atoms linked by (i.e. comprising) one or more double bonds; this includes vinyl, all propenyl-isomers, all butenyl-isomers, all pentenyl-isomers, all hexenyl-isomers, all heptenyl-isomers, all octenyl-isomers, all nonenyl-isomers, all decenyl-isomers, all undecenyl-isomers and all dodecenyl-isomers.

The term “C₂-C₁₂ alkynyl” comprises all isomers of the corresponding unsaturated acetylenic hydrocarbon groups containing two to twelve carbon atoms linked by (i.e. comprising) one or more triple bonds; this includes ethynyl, all propynyl-isomers, all butynyl-isomers, all pentynyl-isomers, all hexynyl-isomers, all heptynyl-isomers, all octynyl-isomers, all nonynyl-isomers, all decynyl-isomers, all undecynyl-isomers and all dodecynyl-isomers. The term “alkynyl” also includes compounds having one or more triple bonds and one or more double bonds.

The term “C₃-C₈ cycloalkyl” comprises the corresponding saturated hydrocarbon groups containing three to eight carbon atoms arranged in a monocyclic ring structure; this includes cyclopropyl, cyclobutyl, cyclopentyl, cyclohexyl, cycloheptyl, cyclooctyl.

The term “C₅-C₈ cycloalkenyl” comprises the corresponding unsaturated non-aromatic and non-heteroaromatic hydrocarbon groups containing five to eight carbon atoms, of which at least one is sp³-hybridized, and which are arranged in a monocyclic ring structure and linked by (i.e. comprising) one or more double bonds; this includes all cyclopentenyl-isomers, all cyclohexenyl-isomers, all cycloheptenyl-isomers, all cyclooctenyl-isomers.

The term “C₅-C₁₂ bicycloalkyl” comprises the corresponding saturated hydrocarbon groups containing five to twelve carbon atoms arranged in a bicyclic ring structure; wherein these bicyclic ring structures include fused, bridged and spiro systems;

The term “C₇-C₁₂ bicycloalkenyl” comprises the corresponding unsaturated non-aromatic and non-heteroaromatic hydrocarbon groups containing seven to twelve carbon atoms arranged in a bicyclic ring structure and linked by (i.e. comprising) one or more double bonds; wherein these bicyclic ring structures include fused, bridged and spiro systems;

The term “C₈-C₁₄ tricycloalkyl” comprises the corresponding saturated hydrocarbon groups containing eight to fourteen carbon atoms arranged in a tricyclic ring structure; wherein these tricyclic ring structures include fused, bridged and spiro systems;

The terms “cyclic”, “bicyclic”, “tricyclic”, “cycloalkyl”, “cycloalkenyl”, “bicycloalkyl”, “bicycloalkenyl” and “tricycloalkyl” for R¹ mean that such cyclic, bicyclic or tricyclic residue is directly linked by a chemical bond to the aromatic ring to which R¹ is bound; and wherein the terms “cyclic”, “bicyclic”, “tricyclic”, “cycloalkyl”, “cycloalkenyl”, “bicycloalkyl”, “bicycloalkenyl” and “tricycloalkyl” for a substituent of R¹ mean that such cyclic, bicyclic or tricyclic residue is directly linked by a chemical bond to one of the C-atoms or N-atoms or O-atoms or S-atoms contained in R¹; e.g. “R¹ is cyclohexyl” means that the cyclohexyl residue is linked to the aromatic ring to which R¹ is bound; and “R¹ is methyl and R¹ is substituted with cyclohexyl” means that the resulting —CH₂(cyclohexyl) residue is linked to the aromatic ring to which R¹ is bound.

In case a carbon atom is replaced by a heteroatom selected from O, N, or S, the number of substituents on the respective heteroatom is adapted according to its valency, e.g. a —CR₂— group may be replaced by a —NR—, —NR₂⁺—, —O— or —S— group.

The term “perhalogenated” relates to the exhaustive halogenation of the carbon scaffold; according residues comprise the corresponding perfluorinated, perchlorinated, perbrominated and periodinated groups. Preferably, the term “perhalogenated” relates to perfluorinated or perchlorinated groups, more preferably to perfluorinated groups.

The following contains definitions of terms used in this specification. The initial definition provided for a group or term herein applies to that group or term throughout the present specification, individually or as part of another group, unless otherwise indicated.

The compounds of the present invention may form salts, which are also within the scope of this invention. Reference to a compound of the invention herein is understood to include reference to salts thereof, unless otherwise indicated. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the substituents comprise an acid moiety such as a carboxyl group and an amino group). Also included herein are quaternary ammonium salts such as alkylammonium salts. Salts of the compounds may be formed, for example, by reacting a compound with an amount of acid or base, such as an equivalent amount, in a medium such as one in which the salt precipitates or in an aqueous medium followed by lyophilization.

Exemplary salts resulting from the addion of acid include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, chlorates, bromates, iodates, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary salts resulting from the addition of base (formed, for example, where the substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, tert-butyl amines, and salts with amino acids such as arginine, lysine and the like. The basic nitrogen-containing groups may be quaternized with agents such as lower alkyl halides (e.g., methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science 1977, 66 (2), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Furthermore, in the case of the compounds of the invention which contain an asymmetric carbon atom or an atropoisomeric bond, the invention relates to the D form, the L form and D,L mixtures and also, where more than one asymmetric carbon atom or atropoisomeric bond is present, to the diastereomeric forms. Those compounds of the invention which contain asymmetric carbon atoms or atropoisomeric bonds, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention can also include all isotopes of atoms occurring in the intermediates or final compounds. Isotopes include those atoms having the same atomic number but different mass numbers. For example, isotopes of hydrogen include tritium and deuterium.

Also included are solvates and hydrates of the compounds of the invention and solvates and hydrates of their pharmaceutically acceptable salts.

The term “compound” as used herein is meant to include all stereoisomers, geometric isomers, tautomers, rotamers, and isotopes of the structures depicted, unless otherwise indicated.

In some embodiments, the compound can be provided as a prodrug. The term “prodrug”, as employed herein, denotes a compound, which, upon administration to a subject, undergoes chemical conversion by metabolic or chemical processes to yield a compound of the invention, or a salt and/or solvate thereof.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof.

Pharmaceutical Methods

The compounds according to the invention have been found to have pharmacologically important properties, which can be used therapeutically. The compounds of the invention can be used alone, in combination with each other or in combination with other active compounds.

In certain embodiments, compounds of the present invention may exhibit growth inhibiting properties in hyperproliferative processes.

The antiproliferative activities of compounds falling under formula (Ia), (Ib) and (Ic), respectively, were investigated on cells or cell lines originating from a disorder of the haematopoietic system, including the myeloid cell compartment and the lymphoid cell compartment (T-cells and B-cells), the neuroendocrine system, the cervix, the breast, the ovaries, the lung, the gastrointestinal tract, and the mucosal epithelium, as well as from the skin epithelium and from the muscle. To this end, HL-60 cells, NB-4 cells, HH cells, RPMI-8402 cells, TANOUE cells, TT cells, HeLa cells, MDA-MB-231 cells, FU-OV-1 cells, LOU-NH91 cells, 23132/87 cells, CAL-27 cells, BHY cells, SCC-25 cells, A-431 cells, human primary epidermal keratinocytes (HPEK), and C2C12 cells were seeded into 96-well plates suitable for fluorescence assays (CORNING #3598) at following initial cell numbers: 1000 cells per well for HL-60; 1000 cells per well for NB-4; 5000 cells per well for HH; 5000 cells per well for RPMI-8402; 1500 cells per well for TANOUE; 9000 cells per well for TT; 2000 cells per well for HeLa; 3000 cells per well for MDA-MB-231; 3000 cells per well for FU-OV-1; 4000 cells per well for LOU-NH91; 2000 cells per well for 23132/87; 2000 cells per well for CAL-27; 1500 cells per well for BHY; 1500 cells per well for SCC-25; 700 cells per well for A-431; 1000 cells per well for HPEK; 500 cells per well for C2C12. The cells were treated with compounds at indicated final concentrations (diluted from the 1000× stock-solutions in DMSO to a final DMSO concentration of 0.1% v/v in H₂O (Water For Injection, WFI, Fisherscientific #10378939)) or with the empty carrier DMSO at 0.1% v/v as control for 5 days. At day 5 after starting the treatments the cells were subjected to the alamarBlue® Proliferation Assay (Bio-Rad Serotec GmbH, BUF012B) according to the protocol of the manufacturer. The readout was taken with a multi-well plate-reader in the fluorescence mode with applying a filter for excitation at 560 nm (band width 10 nm) and for emission at 590 nm (band width 10 nm). Control treatments for growth inhibition with commercial compounds such as Methotrexate (MTREX) and Resveratrol (RES) were included on every plate.

The assays were performed in duplicate or more replicates of independent single experiments each containing a six-fold replicate for every condition. For every individual plate, the measured fluorescence intensity values of the conditions with compound treatment were normalized against the corresponding equally weighted arithmetic mean of the fluorescence intensity values of the six DMSO treated control wells in order to obtain the relative values to a baseline level of 1.0.

Two independent outlier analyses were performed according to the methods by Peirce and Chauvenet (Ross, Journal of Engineering Technology 2003, 1-12). Outliers confirmed by at least one of the methods were excluded from the calculations but not more than one value out of six per compound within a single experiment. The weighted arithmetic mean (here abbreviated as AVE_{w}) for each compound was calculated from the normalized values over all independent replicates of the single experiments comprising the six replicates each. The corresponding standard deviation for the weighted arithmetic mean was calculated according to the method described by Bronstein et al. (Bronstein, Semendjajew, Musiol, Miihlig, Taschenbuch der Mathematik, 5^{th} edition 2001 (German), publisher: Verlag Harri Deutsch, Frankfurt am Main and Thun) and was combined with the Gauß′ error propagation associated with the performed calculation for the normalization. The resulting standard deviation is herein referred to as “combined standard deviation”.

In cases with considerable variation in the normalized equally weighted arithmetic means derived from two independent replicates, the number of independent replicates was increased to three or more. In the cases of four or more independent replicates, a second-line outlier analysis was applied on all normalized equally weighted arithmetic means according to the methods by Peirce and Chauvenet as described above.

In certain embodiments, the compounds of the present invention may be growth inhibitors in hyperproliferative processes, including malignant and non-malignant hyperproliferative processes.

In one embodiment, several compounds of the invention were found to inhibit the growth of HL-60 cells (human acute myeloid leukemia cells) obtainable from the Deutsche Sammlung von Mikroorganismen und Zellkulturen GmbH (DSMZ) under the accession number ACC 3. HL-60 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of HL-60 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of HL-60 cells. The so far identified HL-60 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 29
Proliferation assay with HL-60 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0064 | |
| | 3 | XPF-0065 | |
| | 4 | XPF-0429 | |
| | 5 | XPF-1602 | |
| | 6 | XPF-2250 | |
| 0.6 < AVE_{w} ≤ 0.7 | 7 | XPF-0006 | |
| | 8 | XPF-0266 | |
| | 9 | XPF-0518 | |
| | 10 | XPF-1322 | |
| | 11 | XPF-1542 | |
| | 12 | XPF-2241 | |
| 0.4 < AVE_{w} ≤ 0.6 | 13 | XPF-0062 | |
| | 14 | XPF-0170 | |
| | 15 | XPF-0258 | |
| | 16 | XPF-0496 | |
| | 17 | XPF-1554 | |
| | 18 | XPF-2249 | |
| | 19 | XPF-2253 | |
| 0.4 ± 0.1 | 20 | RES | Control at 20 μM |
| 0.2 < AVE_{w} ≤ 0.4 | 21 | XPF-0014 | |
| | 22 | XPF-0434 | |
| | 23 | XPF-0454 | |
| | 24 | XPF-0469 | |
| | 25 | XPF-1162 | |
| | 26 | XPF-1325 | |
| | 27 | XPF-1588 | |
| | 28 | XPF-1624 | |
| | 29 | XPF-2246 | |
| | 30 | XPF-2248 | |
| | 31 | XPF-2252 | |
| 0.2 ± 0.1 | 32 | RES | Control at 40 μM |
| 0.1 ± 0.1 | 33 | MTREX | Control at 20 μM |
| 0.0 < AVE_{w} ≤ 0.2 | 34 | XPF-0042 | |
| | 35 | XPF-0070 | |
| | 36 | XPF-0174 | |
| | 37 | XPF-0182 | |
| | 38 | XPF-0202 | |
| | 39 | XPF-0210 | |
| | 40 | XPF-0230 | |
| | 41 | XPF-0476 | |
| | 42 | XPF-0504 | |
| | 43 | XPF-0630 | |
| | 44 | XPF-1190 | |
| | 45 | XPF-1196 | |
| | 46 | XPF-1330 | |
| | 47 | XPF-1596 | |
| | 48 | XPF-2242 | |
| | 49 | XPF-2243 | |
| | 50 | XPF-2244 | |
| | 51 | XPF-2245 | |
| | 52 | XPF-2247 | |
| | 53 | XPF-2251 | |
| | 54 | XPF-2254 | |

In one embodiment, several compounds of the invention were found to inhibit the growth of NB-4 cells (human acute promyelocytic leukemia cells) obtainable from the Deutsche Sammlung von Mikroorganismen und Zellkulturen GmbH (DSMZ) under the accession number ACC 207. NB-4 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of NB-4 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib), respectively, have been identified as growth inhibitors of NB-4 cells. The so far identified NB-4 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 30
Proliferation assay with NB-4 cells at 20 μM

| Activity Range | Entry | Compound No. | Specification |
|---|---|---|---|
| 1.0 ± 0.0 | 1 | DMSO | Baseline control |
| 0.7 < AVE_{w} ≤ 0.8 | 2 | XPF-0058 | |
| | 3 | XPF-0469 | |
| | 4 | XPF-2241 | |
| | 5 | XPF-2252 | |
| 0.6 < AVE_{w} ≤ 0.7 | 6 | XPF-0205 | |
| | 7 | XPF-1196 | |
| | 8 | XPF-1554 | |
| | 9 | XPF-1588 | |
| | 10 | XPF-1616 | |
| 0.4 < AVE_{w} ≤ 0.6 | 11 | XPF-1162 | |
| | 12 | XPF-2248 | |
| 0.2 < AVE_{w} ≤ 0.4 | 13 | XPF-0042 | |
| | 14 | XPF-0062 | |
| | 15 | XPF-0202 | |
| | 16 | XPF-1624 | |
| | 17 | XPF-2249 | |
| 0.1 ± 0.0 | 18 | MTREX | Control at 20 μM |
| 0.1 ± 0.0 | 19 | RES | Control at 20 μM |
| 0.0 < AVE_{w} ≤ 0.2 | 20 | XPF-0057 | |
| | 21 | XPF-0070 | |
| | 22 | XPF-0169 | |
| | 23 | XPF-0174 | |
| | 24 | XPF-0182 | |
| | 25 | XPF-0210 | |
| | 26 | XPF-0230 | |
| | 27 | XPF-0426 | |
| | 28 | XPF-0434 | |
| | 29 | XPF-0454 | |
| | 30 | XPF-0476 | |
| | 31 | XPF-0504 | |
| | 32 | XPF-0630 | |
| | 33 | XPF-1190 | |
| | 34 | XPF-1322 | |
| | 35 | XPF-1325 | |
| | 36 | XPF-1330 | |
| | 37 | XPF-1596 | |
| | 38 | XPF-2242 | |
| | 39 | XPF-2243 | |
| | 40 | XPF-2244 | |
| | 41 | XPF-2245 | |
| | 42 | XPF-2246 | |
| | 43 | XPF-2247 | |
| | 44 | XPF-2251 | |
| | 45 | XPF-2254 | |
| 0.0 ± 0.0 | 46 | RES | Control at 40 μM |

In one embodiment, several compounds of the invention were found to inhibit the growth of HH cells (human cutaneous T-cell lymphoma cells) obtainable from the Deutsche Sammlung von Mikroorganismen und Zellkulturen GmbH (DSMZ) under the accession number ACC 707. HH cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of HH cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib), respectively, have been identified as growth inhibitors of HH cells. The so far identified HH growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 31
Proliferation assay with HH cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0057 | |
| | 3 | XPF-0064 | |
| | 4 | XPF-0169 | |
| | 5 | XPF-0426 | |
| | 6 | XPF-0469 | |
| | 7 | XPF-1162 | |
| | 8 | XPF-1196 | |
| | 9 | XPF-2250 | |
| 0.7 < AVE_{w} ≤ 0.8 | 10 | XPF-0230 | |
| | 11 | XPF-0454 | |
| | 12 | XPF-1322 | |
| | 13 | XPF-1554 | |
| | 14 | XPF-1588 | |
| | 15 | XPF-2249 | |
| | 16 | XPF-2253 | |
| 0.6 < AVE_{w} ≤ 0.7 | 17 | XPF-0042 | |
| | 18 | XPF-0062 | |
| | 19 | XPF-0174 | |
| | 20 | XPF-0182 | |
| | 21 | XPF-0434 | |
| | 22 | XPF-2243 | |
| | 23 | XPF-2246 | |
| | 24 | XPF-2248 | |
| | 25 | XPF-2252 | |
| 0.6 ± 0.1 | 26 | RES | Control at 20 μM |
| 0.4 < AVE_{w} ≤ 0.6 | 27 | XPF-0202 | |
| | 28 | XPF-0210 | |
| | 29 | XPF-0476 | |
| | 30 | XPF-2251 | |
| | 31 | XPF-2254 | |
| 0.4 ± 0.1 | 32 | MTREX | Control at 20 μM |
| 0.4 ± 0.1 | 33 | RES | Control at 40 μM |
| 0.2 < AVE_{w} ≤ 0.4 | 34 | XPF-0504 | |
| | 35 | XPF-1190 | |
| | 36 | XPF-1330 | |
| | 37 | XPF-1596 | |
| | 38 | XPF-2245 | |
| | 39 | XPF-2247 | |
| 0.0 < AVE_{w} ≤ 0.2 | 40 | XPF-0070 | |
| | 41 | XPF-0630 | |
| | 42 | XPF-2242 | |
| | 43 | XPF-2244 | |

In one embodiment, several compounds of the invention were found to inhibit the growth of RPMI-8402 cells (human T cell acute lymphoblastic leukemia cells) obtainable from the Deutsche Sammlung von Mikroorganismen und Zellkulturen GmbH (DSMZ) under the accession number ACC 290. RPMI-8402 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of RPMI-8402 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10².

According to the method described above, several molecules falling under the scope of the compounds herein defined in formula (Ia), (Ib) and (Ic), respectively, have been identified as growth inhibitors of RPMI-8402 cells. The so far identified RPMI-8402 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 32
Proliferation assay with RPMI-8402 cells at 20 nM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0422 | |
| | 3 | XPF-1542 | |
| | 4 | XPF-1549 | |
| 0.7 < AVE_{w} ≤ 0.8 | 5 | XPF-0006 | |
| | 6 | XPF-0170 | |
| | 7 | XPF-0426 | |
| | 8 | XPF-0518 | |
| | 9 | XPF-1185 | |
| | 10 | XPF-1616 | |
| 0.6 < AVE_{w} ≤ 0.7 | 11 | XPF-0065 | |
| | 12 | XPF-0205 | |
| | 13 | XPF-0429 | |
| | 14 | XPF-1325 | |
| | 15 | XPF-1624 | |
| | 16 | XPF-2241 | |
| | 17 | XPF-2246 | |
| 0.6 ± 0.0 | 18 | RES | Control at 20 μM |
| 0.4 < AVE_{w} ≤ 0.6 | 19 | XPF-0062 | |
| | 20 | XPF-0169 | |
| | 21 | XPF-0174 | |
| | 22 | XPF-0258 | |
| | 23 | XPF-0266 | |
| | 24 | XPF-0454 | |
| | 25 | XPF-1162 | |
| | 26 | XPF-1196 | |
| | 27 | XPF-1322 | |
| | 28 | XPF-1554 | |
| | 29 | XPF-1588 | |
| | 30 | XPF-2243 | |
| | 31 | XPF-2248 | |
| | 32 | XPF-2250 | |
| | 33 | XPF-2253 | |
| 0.2 < AVE_{w} ≤ 0.4 | 34 | XPF-0064 | |
| | 35 | XPF-0202 | |
| | 36 | XPF-0230 | |
| | 37 | XPF-0434 | |
| | 38 | XPF-0469 | |
| | 39 | XPF-0496 | |
| | 40 | XPF-2245 | |
| | 41 | XPF-2247 | |
| | 42 | XPF-2249 | |
| | 43 | XPF-2252 | |
| 0.1 ± 0.0 | 44 | MTREX | Control at 20 μM |
| 0.1 ± 0.0 | 45 | RES | Control at 40 μM |
| 0.0 < AVE_{w} ≤ 0.2 | 46 | XPF-0042 | |
| | 47 | XPF-0070 | |
| | 48 | XPF-0182 | |
| | 49 | XPF-0210 | |
| | 50 | XPF-0476 | |
| | 51 | XPF-0504 | |
| | 52 | XPF-0630 | |
| | 53 | XPF-1190 | |
| | 54 | XPF-1330 | |
| | 55 | XPF-1596 | |
| | 56 | XPF-2242 | |
| | 57 | XPF-2244 | |
| | 58 | XPF-2251 | |
| | 59 | XPF-2254 | |

In one embodiment, several compounds of the invention were found to inhibit the growth of TANOUE cells (human B cell leukemia cells) obtainable from the Deutsche Sammlung von Mikroorganismen und Zellkulturen GmbH (DSMZ) under the accession number ACC 399.

TANOUE cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of TANOUE cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10².

According to the method described above, several molecules falling under the scope of the compounds herein defined in formula (Ia), (Ib) and (Ic), respectively, have been identified as growth inhibitors of TANOUE cells. The so far identified TANOUE growth inhibitors relate to the compounds listed in Error! Reference source not found. The entries of Error! Reference source not found.are categorized by the corresponding weighted arithmetic means of the compounds without consideration of the respective standard deviations, hence falling into the activity ranges as indicated.

TABLE 33
Proliferation assay with TANOUE cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0064 | |
| | 3 | XPF-0065 | |
| | 4 | XPF-0421 | |
| | 5 | XPF-0422 | |
| | 6 | XPF-0429 | |
| | 7 | XPF-1185 | |
| | 8 | XPF-1542 | |
| 0.7 < AVE_{w} ≤ 0.8 | 9 | XPF-0006 | |
| | 10 | XPF-0170 | |
| | 11 | XPF-1325 | |
| 0.6 < AVE_{w} ≤ 0.7 | 12 | XPF-0057 | |
| | 13 | XPF-0518 | |
| | 14 | XPF-1616 | |
| 0.4 < AVE_{w} ≤ 0.6 | 15 | XPF-0062 | |
| | 16 | XPF-0258 | |
| | 17 | XPF-1624 | |
| 0.2 < AVE_{w} ≤ 0.4 | 18 | XPF-0169 | |
| | 19 | XPF-0266 | |
| | 20 | XPF-0426 | |
| | 21 | XPF-0469 | |
| | 22 | XPF-0496 | |
| | 23 | XPF-1588 | |
| | 24 | XPF-2241 | |
| | 25 | XPF-2243 | |
| | 26 | XPF-2246 | |
| | 27 | XPF-2248 | |
| | 28 | XPF-2250 | |
| | 29 | XPF-2253 | |
| 0.1 ± 0.0 | 30 | MTREX | Control at 20 μM |
| 0.1 ± 0.0 | 31 | RES | Control at 20 μM |
| 0.0 < AVE_{w} ≤ 0.2 | 32 | XPF-0014 | |
| | 33 | XPF-0042 | |
| | 34 | XPF-0070 | |
| | 35 | XPF-0174 | |
| | 36 | XPF-0182 | |
| | 37 | XPF-0202 | |
| | 38 | XPF-0210 | |
| | 39 | XPF-0230 | |
| | 40 | XPF-0434 | |
| | 41 | XPF-0454 | |
| | 42 | XPF-0476 | |
| | 43 | XPF-0504 | |
| | 44 | XPF-0630 | |
| | 45 | XPF-1162 | |
| | 46 | XPF-1190 | |
| | 47 | XPF-1196 | |
| | 48 | XPF-1322 | |
| | 49 | XPF-1330 | |
| | 50 | XPF-1554 | |
| | 51 | XPF-1596 | |
| | 52 | XPF-2242 | |
| | 53 | XPF-2244 | |
| | 54 | XPF-2245 | |
| | 55 | XPF-2247 | |
| | 56 | XPF-2249 | |
| | 57 | XPF-2251 | |
| | 58 | XPF-2252 | |
| | 59 | XPF-2254 | |
| 0.0 ± 0.0 | 60 | RES | Control at 40 μM |

In one embodiment, several compounds of the invention were found to inhibit the growth of TT cells (human medullary thyroid carcinoma cells) obtainable from the American Type Culture Collection (ATCC) under the accession number ATCC-CRL-1803. TT cells were cultivated in F-12K medium (Fisherscientific, #11580556, or ATCC, #ATCC-30-2004) containing 10% fetal bovine serum (Fisherscientific, #15517589) at 37° C. and 5% CO2.

A compound is considered as a growth inhibitor of TT cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), and (Ib), respectively, have been identified as growth inhibitors of TT cells. The so far identified TT growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 34
Proliferation assay with TT cells at 20 μM

| Activity Range | Entry | Compound No. | Specification |
|---|---|---|---|
| 1.0 ± 0.0 | 1 | MTREX | Control at 20 μM |
| 1.0 ± 0.0 | 2 | DMSO | Baseline control |
| 0.9 ± 0.0 | 3 | RES | Control at 20 μM |
| 0.8 < AVE_{w} ≤ 0.9 | 4 | XPF-0014 | |
| | 5 | XPF-0057 | |
| | 6 | XPF-1185 | |
| | 7 | XPF-1325 | |
| 0.7 < AVE_{w} ≤ 0.8 | 8 | XPF-0496 | |
| | 9 | XPF-1330 | |
| | 10 | XPF-1554 | |
| | 11 | XPF-2241 | |
| 0.7 ± 0.0 | 12 | RES | Control at 40 μM |
| 0.6 < AVE_{w} ≤ 0.7 | 13 | XPF-0174 | |
| | 14 | XPF-0426 | |
| | 15 | XPF-1322 | |
| | 16 | XPF-1588 | |
| 0.4 < AVE_{w} ≤ 0.6 | 17 | XPF-0042 | |
| | 18 | XPF-0182 | |
| | 19 | XPF-0469 | |
| | 20 | XPF-2246 | |
| | 21 | XPF-2248 | |
| | 22 | XPF-2249 | |
| | 23 | XPF-2250 | |
| | 24 | XPF-2252 | |
| | 25 | XPF-2253 | |
| 0.2 < AVE_{w} ≤ 0.4 | 26 | XPF-0062 | |
| | 27 | XPF-0169 | |
| | 28 | XPF-0202 | |
| | 29 | XPF-0230 | |
| | 30 | XPF-0454 | |
| | 31 | XPF-1190 | |
| | 32 | XPF-2243 | |
| | 33 | XPF-2254 | |
| 0.0 < AVE_{w} ≤ 0.2 | 34 | XPF-0070 | |
| | 35 | XPF-0210 | |
| | 36 | XPF-0434 | |
| | 37 | XPF-0476 | |
| | 38 | XPF-0504 | |
| | 39 | XPF-0630 | |
| | 40 | XPF-1596 | |
| | 41 | XPF-2242 | |
| | 42 | XPF-2244 | |
| | 43 | XPF-2245 | |
| | 44 | XPF-2247 | |
| | 45 | XPF-2251 | |

In one embodiment, several compounds of the invention were found to inhibit the growth of HeLa cells (human cervical adenocarcinoma cells) obtainable from the American Type Culture Collection (ATCC) under the accession number ATCC-CCL-2. HeLa cells were cultivated in DMEM medium (Fisherscientific, #11584456) containing 10% fetal bovine serum (Fisherscientific, #15517589) at 37° C. and 5% CO2.

A compound is considered as a growth inhibitor of HeLa cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib), respectively, have been identified as growth inhibitors of HeLa cells. The so far identified HeLa growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 35
Proliferation assay with HeLa cells at 20 μM

| Activity Range | Entry | Compound No. | Specification |
|---|---|---|---|
| 1.0 ± 0.0 | 1 | DMSO | Baseline control |
| 0.9 ± 0.0 | 2 | RES | Control at 20 μM |
| 0.8 < AVE_{w} ≤ 0.9 | 3 | XPF-0057 | |
| 0.4 < AVE_{w} ≤ 0.6 | 4 | XPF-0476 | |
| | 5 | XPF-1596 | |
| 0.4 ± 0.1 | 6 | RES | Control at 40 μM |
| 0.4 ± 0.0 | 7 | MTREX | Control at 20 μM |
| 0.0 < AVE_{w} ≤ 0.2 | 8 | XPF-0070 | |
| | 9 | XPF-0630 | |
| | 10 | XPF-1190 | |
| | 11 | XPF-2242 | |
| | 12 | XPF-2244 | |
| | 13 | XPF-2254 | |

In one embodiment, several compounds of the invention were found to inhibit the growth of MDA-MB-231 cells (human breast carcinoma cells) obtainable from the Deutsche Sammlung von Mikroorganismen und Zellkulturen GmbH (DSMZ) under the accession number ACC 732. MDA-MB-231 cells were cultivated in Leibovitz's L-15 (no phenol red) medium (Fisherscientific, #11540556) containing 10% fetal bovine serum (Fisherscientific, #15517589) at 37° C. and 0% CO2.

A compound is considered as a growth inhibitor of MDA-MB-231 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10².

According to the method described above, several molecules falling under the scope of the compounds herein defined in formula (Ia), (Ib) and (Ic), respectively, have been identified as growth inhibitors of MDA-MB-231 cells. The so far identified MDA-MB-231 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 36
Proliferation assay with MDA-MB-231 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-1182 | |
| | 3 | XPF-1546 | |
| | 4 | XPF-1616 | |
| 0.7 < AVE_{w} ≤ 0.8 | 5 | XPF-0063 | |
| | 6 | XPF-0421 | |
| | 7 | XPF-1541 | |
| 0.6 < AVE_{w} ≤0.7 | 8 | XPF-0057 | |
| | 9 | XPF-0170 | |
| | 10 | XPF-0422 | |
| | 11 | XPF-1542 | |
| | 12 | XPF-1549 | |
| 0.6 ± 0.0 | 13 | MTREX | Control at 20 μM |
| 0.6 ± 0.0 | 14 | RES | Control at 20 μM |
| 0.4 < AVE_{w} ≤0.6 | 15 | XPF-0006 | |
| | 16 | XPF-0169 | |
| | 17 | XPF-0205 | |
| | 18 | XPF-0426 | |
| | 19 | XPF-0518 | |
| | 20 | XPF-1185 | |
| | 21 | XPF-2241 | |
| | 22 | XPF-2252 | |
| | 23 | XPF-2253 | |
| 0.2 < AVE_{w} ≤ 0.4 | 24 | XPF-0014 | |
| | 25 | XPF-0062 | |
| | 26 | XPF-0065 | |
| | 27 | XPF-0174 | |
| | 28 | XPF-0230 | |
| | 29 | XPF-0258 | |
| | 30 | XPF-0429 | |
| | 31 | XPF-0434 | |
| | 32 | XPF-0454 | |
| | 33 | XPF-1162 | |
| | 34 | XPF-1196 | |
| | 35 | XPF-1325 | |
| | 36 | XPF-1554 | |
| | 37 | XPF-2243 | |
| | 38 | XPF-2246 | |
| | 39 | XPF-2248 | |
| | 40 | XPF-2250 | |
| | 41 | XPF-2254 | |
| 0.0 < AVE_{w} ≤ 0.2 | 42 | XPF-0042 | |
| | 43 | XPF-0064 | |
| | 44 | XPF-0070 | |
| | 45 | XPF-0182 | |
| | 46 | XPF-0202 | |
| | 47 | XPF-0210 | |
| | 48 | XPF-0266 | |
| | 49 | XPF-0469 | |
| | 50 | XPF-0476 | |
| | 51 | XPF-0496 | |
| | 52 | XPF-0504 | |
| | 53 | XPF-0630 | |
| | 54 | XPF-1190 | |
| | 55 | XPF-1322 | |
| | 56 | XPF-1330 | |
| | 57 | XPF-1588 | |
| | 58 | XPF-1596 | |
| | 59 | XPF-2242 | |
| | 60 | XPF-2244 | |
| | 61 | XPF-2245 | |
| | 62 | XPF-2247 | |
| | 63 | XPF-2249 | |
| | 64 | XPF-2251 | |

In one embodiment, several compounds of the invention were found to inhibit the growth of FU-OV-1 cells (human ovarian carcinoma cells) obtainable from the Deutsche Sammlung von Mikroorganismen und Zellkulturen GmbH (DSMZ) under the accession number ACC 444. FU-OV-1 cells were cultivated in Ham's F-12/DMEM (1:1) medium (Fisherscientific, #11514436) containing 10% fetal bovine serum (Fisherscientific, #15517589) and 1 mM sodium pyruvate (Fisherscientific, #11501871) at 37° C. and 5% CO2.

A compound is considered as a growth inhibitor of FU-OV-1 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1.10-2.

According to the method described above, several molecules falling under the scope of the compounds herein defined in formula (Ia), have been identified as growth inhibitors of FU-OV-1 cells. The so far identified FU-OV-1 growth inhibitors relate to the compounds listed in Error! Reference source not found. The entries of [symbol] are categorized by the corresponding weighted arithmetic means of the compounds without consideration of the respective standard deviations, hence falling into the activity ranges as indicated.

TABLE 37
Proliferation assay with FU-OV-1 cells at 20 μM

| Activity Range | Entry | Compound No. | Specification |
|---|---|---|---|
| 1.0 ± 0.0 | 1 | MTREX | Control at 20 μM |
| 1.0 ± 0.0 | 2 | DMSO | Baseline control |
| 0.7 ± 0.0 | 3 | RES | Control at 20 μM |
| 0.4 < AVE_{w} ≤ 0.6 | 4 | XPF-1325 | |
| 0.4 ± 0.0 | 5 | RES | Control at 40 μM |
| 0.0 < AVE_{w} ≤ 0.2 | 6 | XPF-0630 | |
| | 7 | XPF-2242 | |
| | 8 | XPF-2244 | |

In one embodiment, several compounds of the invention were found to inhibit the growth of LOU-NH91 cells (human lung squamous cell carcinoma cells) obtainable from the Deutsche Sammlung von Mikroorganismen und Zellkulturen GmbH (DSMZ) under the accession number ACC 393. LOU-NH91 cells were cultivated in RPMI 1640 medium (Fisherscientific, #11554526) containing 10% fetal bovine serum (Fisherscientific, #15517589) at 37° C. and 5% CO₂.

A compound is considered as a growth inhibitor of LOU-NH91 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib), respectively, have been identified as growth inhibitors of LOU-NH91 cells. The so far identified LOU-NH91 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 38
Proliferation assay with LOU-NH91 cells at 20 μM

| Activity Range | Entry | Compound No. | Specification |
|---|---|---|---|
| 1.0 ± 0.0 | 1 | DMSO | Baseline control |
| 0.9 ± 0.1 | 2 | RES | Control at 20 μM |
| 0.8 < AVE_{w} ≤ 0.9 | 3 | XPF-0202 | |
| | 4 | XPF-0266 | |
| | 5 | XPF-0422 | |
| | 6 | XPF-0454 | |
| | 7 | XPF-2248 | |
| | 8 | XPF-2252 | |
| 0.7 < AVE_{w} ≤ 0.8 | 9 | XPF-0170 | |
| | 10 | XPF-0182 | |
| | 11 | XPF-0434 | |
| | 12 | XPF-1162 | |
| | 13 | XPF-1596 | |
| | 14 | XPF-2243 | |
| 0.7 ± 0.0 | 15 | RES | Control at 40 μM |
| 0.6 < AVE_{w} ≤ 0.7 | 16 | XPF-2249 | |
| 0.5 ± 0.1 | 17 | MTREX | Control at 20 μM |
| 0.4 < AVE_{w} ≤ 0.6 | 18 | XPF-0042 | |
| | 19 | XPF-2251 | |
| 0.2 < AVE_{w} ≤ 0.4 | 20 | XPF-0210 | |
| 0.0 < AVE_{w} ≤ 0.2 | 21 | XPF-0070 | |
| | 22 | XPF-0230 | |
| | 23 | XPF-0476 | |
| | 24 | XPF-0504 | |
| | 25 | XPF-0630 | |
| | 26 | XPF-1190 | |
| | 27 | XPF-2242 | |
| | 28 | XPF-2244 | |
| | 29 | XPF-2245 | |
| | 30 | XPF-2247 | |

In one embodiment, several compounds of the invention were found to inhibit the growth of 23132/87 cells (human gastric adenocarcinoma cells) obtainable from the Deutsche Sammlung von Mikroorganismen und Zellkulturen GmbH (DSMZ) under the accession number ACC 201. 23132/87 cells were cultivated in RPMI 1640 medium (Fisherscientific, #11554526) containing 10% fetal bovine serum (Fisherscientific, #15517589) at 37° C. and 5% CO2.

A compound is considered as a growth inhibitor of 23132/87 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib) respectively, have been identified as growth inhibitors of 23132/87 cells. The so far identified 23132/87 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 39
Proliferation assay with 23132/87 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0258 | |
| | 3 | XPF-1162 | |
| | 4 | XPF-1616 | |
| | 5 | XPF-2250 | |
| | 6 | XPF-2253 | |
| 0.7 < AVE_{w} ≤ 0.8 | 7 | XPF-0454 | |
| | 8 | XPF-2246 | |
| 0.6 < AVE_{w} ≤0.7 | 9 | XPF-0169 | |
| | 10 | XPF-0496 | |
| 0.5 ± 0.1 | 11 | RES | Control at 20 μM |
| 0.4 < AVE_{w} ≤0.6 | 12 | XPF-0014 | |
| | 13 | XPF-0042 | |
| | 14 | XPF-0182 | |
| | 15 | XPF-0202 | |
| | 16 | XPF-1196 | |
| | 17 | XPF-2241 | |
| | 18 | XPF-2243 | |
| | 19 | XPF-2248 | |
| | 20 | XPF-2252 | |
| 0.3 ± 0.0 | 21 | MTREX | Control at 20 μM |
| 0.2 < AVE_{w} ≤ 0.4 | 22 | XPF-0230 | |
| | 23 | XPF-0434 | |
| | 24 | XPF-0469 | |
| | 25 | XPF-1330 | |
| | 26 | XPF-2245 | |
| | 27 | XPF-2247 | |
| | 28 | XPF-2249 | |
| 0.2 ± 0.0 | 29 | RES | Control at 40 μM |
| 0.0 < AVE_{w} ≤ 0.2 | 30 | XPF-0070 | |
| | 31 | XPF-0210 | |
| | 32 | XPF-0476 | |
| | 33 | XPF-0504 | |
| | 34 | XPF-0630 | |
| | 35 | XPF-1190 | |
| | 36 | XPF-1554 | |
| | 37 | XPF-1596 | |
| | 38 | XPF-2242 | |
| | 39 | XPF-2244 | |
| | 40 | XPF-2251 | |
| | 41 | XPF-2254 | |

In one embodiment, several compounds of the invention were found to inhibit the growth of CAL-27 cells (human tongue squamous cell carcinoma cells) obtainable from the Deutsche Sammlung von Mikroorganismen und Zellkulturen GmbH (DSMZ) under the accession number ACC 446. CAL-27 cells were cultivated in DMEM medium (Fisherscientific, #11584456) containing 10% fetal bovine serum (Fisherscientific, #15517589) at 37° C. and 5% CO₂.

A compound is considered as a growth inhibitor of CAL-27 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of CAL-27 cells. The so far identified CAL-27 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 40
Proliferation assay with CAL-27 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0006 | |
| | 3 | XPF-0258 | |
| | 4 | XPF-2250 | |
| 0.8 ± 0.1 | 5 | RES | Control at 20 μM |
| 0.7 < AVE_{w} ≤ 0.8 | 6 | XPF-0064 | |
| | 7 | XPF-0170 | |
| | 8 | XPF-0426 | |
| | 9 | XPF-0496 | |
| | 10 | XPF-0518 | |
| 0.6 < AVE_{w} ≤ 0.7 | 11 | XPF-0014 | |
| | 12 | XPF-0454 | |
| | 13 | XPF-0469 | |
| | 14 | XPF-2243 | |
| | 15 | XPF-2246 | |
| | 16 | XPF-2248 | |
| 0.4 < AVE_{w} ≤ 0.6 | 17 | XPF-0057 | |
| | 18 | XPF-0062 | |
| | 19 | XPF-0169 | |
| | 20 | XPF-0266 | |
| | 21 | XPF-1322 | |
| | 22 | XPF-1325 | |
| 0.4 ± 0.3 | 23 | RES | Control at 40 μM |
| 0.2 < AVE_{w} ≤ 0.4 | 24 | XPF-0042 | |
| | 25 | XPF-0174 | |
| | 26 | XPF-0182 | |
| | 27 | XPF-0434 | |
| | 28 | XPF-1196 | |
| | 29 | XPF-1588 | |
| | 30 | XPF-2245 | |
| | 31 | XPF-2247 | |
| 0.1 ± 0.0 | 32 | MTREX | Control at 20 μM |
| 0.0 < AVE_{w} ≤ 0.2 | 33 | XPF-0070 | |
| | 34 | XPF-0202 | |
| | 35 | XPF-0210 | |
| | 36 | XPF-0230 | |
| | 37 | XPF-0476 | |
| | 38 | XPF-0504 | |
| | 39 | XPF-0630 | |
| | 40 | XPF-1190 | |
| | 41 | XPF-1330 | |
| | 42 | XPF-1554 | |
| | 43 | XPF-1596 | |
| | 44 | XPF-2242 | |
| | 45 | XPF-2244 | |
| | 46 | XPF-2251 | |
| | 47 | XPF-2254 | |

In one embodiment, several compounds of the invention were found to inhibit the growth of BHY cells (human oral squamous cell carcinoma cells) obtainable from the Deutsche Sammlung von Mikroorganismen und Zellkulturen GmbH (DSMZ) under the accession number ACC 404. BHY cells were cultivated in DMEM medium (Fisherscientific, #11584456) containing 10% fetal bovine serum (Fisherscientific, #15517589) at 37° C. and 5% CO₂.

A compound is considered as a growth inhibitor of BHY cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib), respectively, have been identified as growth inhibitors of BHY cells. The so far identified BHY growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 41
Proliferation assay with BHY cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0266 | |
| | 3 | XPF-0426 | |
| | 4 | XPF-0454 | |
| | 5 | XPF-2249 | |
| 0.7 < AVE_{w} ≤ 0.8 | 6 | XPF-0058 | |
| | 7 | XPF-0062 | |
| | 8 | XPF-0064 | |
| | 9 | XPF-1196 | |
| 0.6 < AVE_{w} ≤ 0.7 | 10 | XPF-0170 | |
| | 11 | XPF-0174 | |
| 0.5 ± 0.1 | 12 | RES | Control at 20 μM |
| 0.4 < AVE_{w} ≤ 0.6 | 13 | XPF-0042 | |
| | 14 | XPF-0057 | |
| | 15 | XPF-0169 | |
| | 16 | XPF-0182 | |
| | 17 | XPF-0434 | |
| | 18 | XPF-1322 | |
| | 19 | XPF-1325 | |
| | 20 | XPF-1588 | |
| | 21 | XPF-2245 | |
| 0.3 ± 0.0 | 22 | MTREX | Control at 20 μM |
| 0.3 ± 0.0 | 23 | RES | Control at 40 μM |
| 0.2 < AVE_{w} ≤ 0.4 | 24 | XPF-0202 | |
| | 25 | XPF-0230 | |
| | 26 | XPF-1330 | |
| | 27 | XPF-1554 | |
| | 28 | XPF-2247 | |
| | 29 | XPF-2251 | |
| 0.0 < AVE_{w} ≤ 0.2 | 30 | XPF-0070 | |
| | 31 | XPF-0210 | |
| | 32 | XPF-0476 | |
| | 33 | XPF-0504 | |
| | 34 | XPF-0630 | |
| | 35 | XPF-1190 | |
| | 36 | XPF-1596 | |
| | 37 | XPF-2242 | |
| | 38 | XPF-2244 | |
| | 39 | XPF-2254 | |

In one embodiment, several compounds of the invention were found to inhibit the growth of SCC-25 cells (human tongue squamous cell carcinoma cells) obtainable from the Deutsche Sammlung von Mikroorganismen und Zellkulturen GmbH (DSMZ) under the accession number ACC 617. SCC-25 cells were cultivated in Ham's F-12/DMEM (1:1) medium (Fisherscientific, #11514436) containing 10% fetal bovine serum (Fisherscientific, #15517589) and 1 mM sodium pyruvate (Fisherscientific, #11501871) at 37° C. and 5% CO₂.

A compound is considered as a growth inhibitor of SCC-25 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10₋₂.

According to the method described above, several molecules falling under the scope of the compounds herein defined in formula (Ia) and (Ib), respectively, have been identified as growth inhibitors of SCC-25 cells. The so far identified SCC-25 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 42
Proliferation assay with SCC-25 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0057 | |
| | 3 | XPF-0169 | |
| | 4 | XPF-0174 | |
| | 5 | XPF-0454 | |
| | 6 | XPF-0476 | |
| | 7 | XPF-2253 | |
| 0.7 < AVE_{w} ≤ 0.8 | 8 | XPF-0014 | |
| | 9 | XPF-0042 | |
| | 10 | XPF-0496 | |
| | 11 | XPF-1196 | |
| | 12 | XPF-2252 | |
| 0.6 < AVE_{w} ≤ 0.7 | 13 | XPF-0202 | |
| | 14 | XPF-0469 | |
| | 15 | XPF-1588 | |
| 0.5 ± 0.1 | 16 | MTREX | Control at 20 μM |
| 0.4 ± 0.1 | 17 | RES | Control at 20 μM |
| 0.2 < AVE_{w} ≤ 0.4 | 18 | XPF-1322 | |
| | 19 | XPF-2249 | |
| 0.1 ± 0.0 | 20 | RES | Control at 40 μM |
| 0.0 < AVE_{w} ≤ 0.2 | 21 | XPF-0070 | |
| | 22 | XPF-0182 | |
| | 23 | XPF-0210 | |
| | 24 | XPF-0230 | |
| | 25 | XPF-0504 | |
| | 26 | XPF-0630 | |
| | 27 | XPF-1190 | |
| | 28 | XPF-1325 | |
| | 29 | XPF-1596 | |
| | 30 | XPF-2242 | |
| | 31 | XPF-2244 | |
| | 32 | XPF-2245 | |
| | 33 | XPF-2247 | |
| | 34 | XPF-2251 | |
| | 35 | XPF-2254 | |

In one embodiment, several compounds of the invention were found to inhibit the growth of A-431 cells (human epidermoid squamous cell carcinoma cells) obtainable from the Cell Lines Service GmbH (CLS) under the accession number 300112. A-431 cells were cultivated in DMEM medium (Fisherscientific, #11584456) containing 10% fetal bovine serum (Fisherscientific, #15517589) at 37° C. and 5% CO2.

A compound is considered as a growth inhibitor of A-431 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia) and (Ib), respectively, have been identified as growth inhibitors of A-431 cells. The so far identified A-431 growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 43
Proliferation assay with A-431 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0058 | |
| | 3 | XPF-0258 | |
| | 4 | XPF-1616 | |
| 0.7 < AVE_{w} ≤ 0.8 | 5 | XPF-0006 | |
| | 6 | XPF-0014 | |
| | 7 | XPF-0496 | |
| | 8 | XPF-1542 | |
| | 9 | XPF-1624 | |
| | 10 | XPF-2243 | |
| | 11 | XPF-2245 | |
| 0.6 < AVE_{w} ≤ 0.7 | 12 | XPF-0266 | |
| | 13 | XPF-0434 | |
| | 14 | XPF-1330 | |
| | 15 | XPF-1588 | |
| | 16 | XPF-2247 | |
| | 17 | XPF-2248 | |
| 0.6 ± 0.1 | 18 | RES | Control at 20 μM |
| 0.4 < AVE_{w} ≤ 0.6 | 19 | XPF-0057 | |
| | 20 | XPF-0062 | |
| | 21 | XPF-0064 | |
| | 22 | XPF-0169 | |
| | 23 | XPF-0469 | |
| | 24 | XPF-1196 | |
| | 25 | XPF-1322 | |
| | 26 | XPF-1325 | |
| | 27 | XPF-2252 | |
| 0.2 < AVE_{w} ≤ 0.4 | 28 | XPF-0042 | |
| | 29 | XPF-0174 | |
| | 30 | XPF-2249 | |
| 0.2 ± 0.1 | 31 | MTREX | Control at 20 μM |
| 0.2 ± 0.0 | 32 | RES | Control at 40 μM |
| 0.0 < AVE_{w} ≤ 0.2 | 33 | XPF-0070 | |
| | 34 | XPF-0182 | |
| | 35 | XPF-0202 | |
| | 36 | XPF-0210 | |
| | 37 | XPF-0230 | |
| | 38 | XPF-0476 | |
| | 39 | XPF-0504 | |
| | 40 | XPF-0630 | |
| | 41 | XPF-1190 | |
| | 42 | XPF-1554 | |
| | 43 | XPF-1596 | |
| | 44 | XPF-2242 | |
| | 45 | XPF-2244 | |
| | 46 | XPF-2251 | |
| | 47 | XPF-2254 | |

In one embodiment, several compounds of the invention were found to inhibit the growth of human epidermal keratinocyte progenitors, (HPEKp, pooled), obtainable from CELLnTEC Advanced Cell Systems AG under the accession number HPEKp. HPEKp cells were cultivated in CnT-Prime epithelial culture medium (CELLnTEC, #CnT-PR, a fully defined, low calcium formulation, completely free of animal or human-derived components) without addition of further components at 37° C. and 5% CO₂.

A compound is considered as a growth inhibitor of HPEKp cells, if—at a reference concentration of 10 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10⁻².

According to the method described above, several molecules falling under the scope of the compounds herein defined in formula (Ia), (Ib) and (Ic), respectively, have been identified as growth inhibitors of HPEKp cells. The so far identified HPEKp growth inhibitors relate to the compounds listed in Error! Reference source not found. The entries of Error! Reference source not found. are categorized by the corresponding weighted arithmetic means of the compounds without consideration of the respective standard deviations, hence falling into the activity ranges as indicated.

TABLE 44
Proliferation assay with HPEKp cells at 10 μM

| Activity Range | Entry | Compound No. | Specification |
|---|---|---|---|
| 1.0 ± 0.0 | 1 | DMSO | Baseline control |
| 0.9 ± 0.0 | 2 | MTREX | Control at 20 μM |
| 0.8 < AVE_{w} ≤ 0.9 | 3 | XPF-0065 | |
| | 4 | XPF-1616 | |
| 0.7 < AVE_{w} ≤ 0.8 | 5 | XPF-0422 | |
| | 6 | XPF-1178 | |
| | 7 | XPF-1624 | |
| 0.6 < AVE_{w} ≤ 0.7 | 8 | XPF-1546 | |
| 0.4 < AVE_{w} ≤ 0.6 | 9 | XPF-0058 | |
| | 10 | XPF-0258 | |
| | 11 | XPF-0421 | |
| | 12 | XPF-1541 | |
| | 13 | XPF-1542 | |
| 0.2 < AVE_{w} ≤ 0.4 | 14 | XPF-0518 | |
| 0.2 ± 0.0 | 15 | RES | Control at 20 μM |
| 0.2 ± 0.0 | 16 | RES | Control at 40 μM |
| 0.0 < AVE_{w} ≤ 0.2 | 17 | XPF-0014 | |
| | 18 | XPF-0042 | |
| | 19 | XPF-0057 | |
| | 20 | XPF-0062 | |
| | 21 | XPF-0064 | |
| | 22 | XPF-0070 | |
| | 23 | XPF-0169 | |
| | 24 | XPF-0170 | |
| | 25 | XPF-0174 | |
| | 26 | XPF-0182 | |
| | 27 | XPF-0202 | |
| | 28 | XPF-0210 | |
| | 29 | XPF-0230 | |
| | 30 | XPF-0266 | |
| | 31 | XPF-0426 | |
| | 32 | XPF-0434 | |
| | 33 | XPF-0454 | |
| | 34 | XPF-0469 | |
| | 35 | XPF-0476 | |
| | 36 | XPF-0496 | |
| | 37 | XPF-0504 | |
| | 38 | XPF-0630 | |
| | 39 | XPF-1162 | |
| | 40 | XPF-1190 | |
| | 41 | XPF-1196 | |
| | 42 | XPF-1322 | |
| | 43 | XPF-1330 | |
| | 44 | XPF-1554 | |
| | 45 | XPF-1588 | |
| | 46 | XPF-1596 | |
| | 47 | XPF-2241 | |
| | 48 | XPF-2242 | |
| | 49 | XPF-2243 | |
| | 50 | XPF-2244 | |
| | 51 | XPF-2245 | |
| | 52 | XPF-2246 | |
| | 53 | XPF-2247 | |
| | 54 | XPF-2248 | |
| | 55 | XPF-2249 | |
| | 56 | XPF-2250 | |
| | 57 | XPF-2251 | |
| | 58 | XPF-2252 | |
| | 59 | XPF-2253 | |
| | 60 | XPF-2254 | |

In one embodiment, several compounds of the invention were found to inhibit the growth of C2C12 cells (murine myoblast cells) obtainable from the Deutsche Sammlung von Mikroorganismen und Zellkulturen GmbH (DSMZ) under the accession number ACC 565. C2C12 cells were cultivated in RPMI 1640 medium (Fisherscientific, #11554526) containing 10% fetal bovine serum (Fisherscientific, #15517589) at 37° C. and 5% CO2.

A compound is considered as a growth inhibitor of C2C12 cells, if—at a reference concentration of 20 μM—the weighted arithmetic mean of the normalized fluorescence intensity values after addition of the corresponding combined standard deviation amounts to 0.9 or lower, in particular to 0.8 or lower, 0.7 or lower, 0.6 or lower, 0.4 or lower, and 0.2 or lower, relative to the overall basis level of 1.0. The overall basis level was calculated as the weighted arithmetic mean of all normalized values from the DMSO control measurements in analogy to the calculations performed for the test-compounds. The corresponding combined standard deviation for the DMSO values amounts to less than 1·10².

According to the method described above, several molecules falling under the scope of the compounds herein defined in formula (Ia) and (Ib), respectively, have been identified as growth inhibitors of C2C12 cells. The so far identified C2C12 growth inhibitors relate to the compounds listed inError! Reference source not found. The entries of Error! Reference source not found. are categorized by the corresponding weighted arithmetic means of the compounds without consideration of the respective standard deviations, hence falling into the activity ranges as indicated.

TABLE 45
Proliferation assay with C2C12 cells at 20 μM

| Activity Range | Entry | Compound No. | Specification |
|---|---|---|---|
| 1.0 ± 0.0 | 1 | DMSO | Baseline control |
| 0.8 < AVE_{w} ≤ 0.9 | 2 | XPF-0064 | |
| | 3 | XPF-0174 | |
| | 4 | XPF-0454 | |
| | 5 | XPF-0469 | |
| | 6 | XPF-1196 | |
| | 7 | XPF-2241 | |
| | 8 | XPF-2243 | |
| | 9 | XPF-2248 | |
| | 10 | XPF-2250 | |
| 0.8 ± 0.0 | 11 | RES | Control at 20 μM |
| 0.7 < AVE_{w} ≤ 0.8 | 12 | XPF-0062 | |
| | 13 | XPF-0434 | |
| 0.6 < AVE_{w} ≤ 0.7 | 14 | XPF-1322 | |
| | 15 | XPF-2249 | |
| 0.4 < AVE_{w} ≤ 0.6 | 16 | XPF-0006 | |
| | 17 | XPF-0202 | |
| | 18 | XPF-1190 | |
| | 19 | XPF-1325 | |
| | 20 | XPF-1330 | |
| | 21 | XPF-2253 | |
| 0.2 < AVE_{w} ≤ 0.4 | 22 | XPF-0210 | |
| 0.2 ± 0.1 | 23 | RES | Control at 40 μM |
| 0.1 ± 0.0 | 24 | MTREX | Control at 20 μM |
| 0.0 < AVE_{w} ≤ 0.2 | 25 | XPF-0070 | |
| | 26 | XPF-0182 | |
| | 27 | XPF-0230 | |
| | 28 | XPF-0476 | |
| | 29 | XPF-0504 | |
| | 30 | XPF-0630 | |
| | 31 | XPF-1596 | |
| | 32 | XPF-2242 | |
| | 33 | XPF-2244 | |
| | 34 | XPF-2245 | |
| | 35 | XPF-2247 | |
| | 36 | XPF-2251 | |
| | 37 | XPF-2252 | |
| | 38 | XPF-2254 | |

In one aspect, the present invention relates to the treatment of skin, skin appendages, mucosa, mucosal appendages, cornea, and all kinds of epithelial tissue. The term “skin” relates to tissue including epidermis and dermis. The term “mucosa” relates to mucous and submucous tissues including oral mucosa, nasal mucosa, ocular mucosa, mucosa of the ear, respiratory mucosa, genital mucosa, urothelial mucosa, anal mucosa and rectal mucosa. The term “appendages” relates to tissue including hair follicles, hair, fingernails, toenails and glands including sebaceous glands, sweat glands, e.g. apocrine or eccrine sweat glands and mammary glands.

In one embodiment, the present invention relates to treatment of non-melanoma skin cancer and pre-cancerous lesions, such as basal cell carcinoma (BCC), squamous cell carcinoma (SCC), sebaceous gland carcinoma, Merkel cell carcinoma, angiosarcoma, cutaneous B-cell lymphoma, cutaneous T-cell lymphoma, dermatofibrosarcoma, actinic keratosis (AK) or Bowen's disease (BD), and cancer and pre-cancerous lesions of other squamous epithelia e.g. cutaneous SCC, lung SCC, head and neck SCC, oral SCC, tongue SCC, esophageal SCC, cervical SCC, periocular SCC, SCC of the thyroid, SCC of the penis, SCC of the vagina, SCC of the prostate and SCC of the bladder.

In a further embodiment, the present invention relates to the treatment of skin and mucosal disorders with cornification defects (keratoses) and/or abnormal keratinocyte proliferation, such as Psoriasis, Darier's disease, Lichen planus, Lupus erythematosus, Ichthyosis or Verruca vulgaris (senilis).

In a further embodiment, the invention relates to the treatment of skin and mucosal diseases, and skin and mucosal cancer each related to and/or caused by viral infections, such as warts, and warts related to HPV (human papilloma virus), papillomas, HPV-related papillomas, papillomatoses and HPV-related papillomatoses, e.g. Verruca (plantar warts), Verruca plana (flat warts/plane warts), Verruca filiformis (filiform warts), mosaic warts, periungual warts, subungual warts, oral warts, genital warts, fibroepithelial papilloma, intracanalicular papilloma, intraductal papilloma, inverted papilloma, basal cell papilloma, squamous papilloma, cutaneous papilloma, fibrovasular papilloma, plexus papilloma, nasal papilloma, pharyngeal papilloma, Papillomatosis cutis carcinoides, Papillomatosis cutis lymphostatica, Papillomatosis confluens et reticularis or laryngeal papillomatosis (respiratory papillomatosis), Herpes-related diseases, e.g. Herpes labialis, Herpes genitalis, Herpes zoster, Herpes corneae or Kaposi's sarcoma and HPV-related cancer of the cervix, vulva, penis, vagina, anus, oropharynx, tongue and oral cavity.

In a further embodiment, the invention relates to the treatment of atopic dermatitis.

In a further embodiment, the invention relates to the treatment of acne.

In a further embodiment, the invention relates to the treatment of wounds of the skin, wherein the process of wound healing is accelerated.

In a further embodiment, the invention relates to the treatment of cancer related to and/or caused by viral infections, i.e. oncoviral infections, e.g. cancer related to HBV- and HCV (hepatitis virus B and C) such as liver cancer, cancer related to EBV (Epstein-Barr virus) such as Burkitt lymphoma, Hodgkin's and non-Hodgkin's lymphoma and stomach cancer, cancer related to HPV (human papilloma virus) such as cervical cancer, cancer related to HHV (human herpes virus) such as Kaposi's sarcoma, and cancer related to HTLV (human T-lymphotrophic virus) such as T-cell leukemia and T-cell lymphoma.

A further aspect of the present invention relates to the treatment of immune system-related disorders. The term “immune system-related disorders” as used herein applies to a pathological condition of the haematopoietic system including the haematologic system, in particular a pathological condition of immune cells belonging to the inate or adaptive immune system.

Examples are diseases of the haematopoietic system including the haematologic system, such as malignancies of the myeloid lineage including acute and chronic forms of leukemia, e.g. chronic myelomonocytic leukemia (CMML), acute myeloid leukemia (AML), and acute promyelocytic leukemia (APL); or malignancies of the lymphoid lineage including acute and chronic forms of leukemia and lymphoma, e.g. T-cell acute lymphoblastic leukemia (T-ALL), pre-T-cell acute lymphoblastic leukemia (pre-T-ALL), cutaneous T-cell lymphoma, chronic lymphocytic leukemia (CLL) including T-cell-CLL (T-CLL) and B-cell-CLL (B-CLL), prolymphocytic leukemia (PLL) including T-cell-PLL (T-PLL) and B-cell-PLL (B-PLL), B-cell acute lymphoblastic leukemia (B-ALL), pre-B-cell acute lymphoblastic leukemia (pre-B-ALL), cutaneous B-cell lymphoma, Hodgkin lymphoma, non-Hodgkin lymphoma, mantle cell lymphoma, myeloma or multiple myeloma; or acute lymphoblastic and acute myeloid mixed lineage leukemia with MLL gene translocation.

A further aspect of the present invention relates to the therapeutic use in immune system-related applications. The term “immune system-related application” as used herein applies to the intervention into proliferation, differentiation and/or activation of cell lineages of the haematopoietic system including the haematologic system in order to modulate an immune response (immune modulation). The term “immune system-related application” as used herein also applies to the intervention into the cellular and non-cellular microenvironment of sites of action of immune cells in order to support and/or enable immune cells in their performance. In particular, the interventions as here defined with the term “immune system-related application” relate to immune cells belonging to the inate or adaptive immune system.

Thus, the compounds of the invention may be used in immunotherapy, alone or together with other immunotherapeutic methods or compounds, as immunologic adjuvant, e.g. as vaccine adjuvant, or as adjuvant for immunotherapy. The term “immunotherapy” as used herein applies to activation-immunotherapy in patients without immune deficiency or with acquired or congenital immune deficiency, and as immune recovery to enhance the functionality of the immune system in the response against pathogens or pathologically transformed endogenous cells, such as cancer cells.

The term “other immunotherapy methods” as used herein applies to vaccinations, antibody treatment, cytokine therapy, the use of immune checkpoint inhibitors and immune response-stimulating drugs, as well as to autologous transplantations of genetically modified or non-modified immune cells, which may be stimulated with intercellular signals, or signaling molecules, or antigens, or antibodies, i.e. adoptive immune-cell transfer.

The method of use of the present invention in immune system-related applications and other immunotherapy methods relates to the use in vivo, in vitro, and ex vivo, respectively.

Specific examples are activation and/or enhancement of activation of peripheral T-lymphocytes, including T-helper cells and cytotoxic T-cells, in order to amplify an immune response, particularly the stimulation of proliferation and/or production and/or secretion of cytokines and/or cytotoxic agents upon antigen recognition in order to amplify an immune response; and the activation and/or enhancement of activation of B-lymphocytes in order to amplify an immune response, particularly the stimulation of proliferation and/or antibody production and/or secretion; and the enhancement of an immune response through augmentation of the number of specific immune-cell subtypes, by regulation of differentiation and/or cell fate decision during immune-cell development, as for example to regulate, particularly to augment the number of immune cells belonging to the T- and B-cell lineage, including marginal zone B-cells, cytotoxic T-cells or T-helper (Th) subsets in particular Th1, Th2, Th17 and regulatory T-cells; or the use as immunologic adjuvant such as vaccine adjuvant.

A still further aspect of the invention relates to the treatment of muscular diseases including diseases of skeletal muscle, cardiac muscle and smooth muscle.

In one embodiment, the invention relates to the treatment of muscular dystrophies (MD).

Specific examples are Duchenne MD, Becker MD, congenital MD, Limb-Girdle MD, facioscapulohumeral MD, Emery-Dreifuss MD, distal MD, myotonic MD or oculopharyngeal MD.

In a further embodiment, the invention relates to the treatment of hyperproliferative disorders of the muscle, including myoblastoma, rhabdomyoma, and rhabdomyosarcoma, as well as muscle hyperplasia and muscle hypertrophy.

In a further embodiment, the compounds of the invention may be used for muscle regeneration after pathologic muscle degeneration or atrophy, e.g. caused by traumata, caused by muscle ischemia or caused by inflammation, in aging-related muscle-atrophy or in disease-related muscle atrophy such as myositis and fibromyositis or poliomyelitis.

A still further aspect relates to the treatment of disorders of the neuroendocrine system such as cancer of the neuroendocrine system, comprising neuroendocrine small cell carcinomas, neuroendocrine large cell carcinomas and carcinoid tumors, e.g. of the brain, thyroid, pancreas, gastrointestinal tract, liver, esophagus, and lung, such as neuroendocrine tumor of the pituitary gland, neuroendocrine tumor of the adrenal gland, medullary thyroid cancer (MTC), C-cell hyperplasia, anaplastic thyroid cancer (ATC), parathyroid adenoma, intrathyroidal nodules, insular carcinoma, hyalinizing trabecular neoplasm, paraganglioma, lung carcinoid tumors, neuroblastoma, gastrointestinal carcinoid, Goblet-cell carcinoid, pancreatic carcinoid, gastrinoma, glucagenoma, somatostatinoma, VIPoma, insulinoma, non-functional islet cell tumor, multiple endocrine neoplasia type-1, or pulmonary carcinoid.

A still further aspect relates to the treatment of disorders of the lung such as cancer of the lung, comprising small-cell lung cancer (SCLC) and non-small-cell lung cancer (NSCLC), including lung squamous cell carcinoma, lung adenocarcinoma and lung large cell carcinoma.

A still further aspect relates to the treatment of hyperproliferative diseases, cancers or pre-cancerous lesions of the brain, pancreas, breast, ovaries, liver, thyroid, genitourinary tract, gastrointestinal tract, and endothelial tissue, including glioma, mixed glioma, glioblastoma multiforme, astrocytoma, anaplastic astrocytoma, glioblastoma, oligodendroglioma, anaplastic oligodendroglioma, anaplastic oligoastrocytoma, ependymoma, anaplastic ependymoma, myxopapillary ependymoma, subependymoma, brain stem glioma, optic nerve glioma, and forebrain tumors, pancreatic adenocarcinoma, pancreatic ductal adenocarcinoma, pancreatic acinar cell carcinoma, pancreatic pseudopapillary neoplasm, pancreatic intraductal papillary-mucinous neoplasm, pancreatic mucinous cystadenocarcinoma, pancreatoblastoma and pancreatic intraepithelial neoplesia, hepatocellular carcinoma, fibrolamellar hepatocellular carcinoma, papillary thyroid cancer and follicular thyroid cancer, cervical cancer, hormone receptor-positive breast cancer and hormone receptor-negative breast cancer, ovarian cancer, gastric cancer and angiosarcoma.

The method of use of the present invention relates to the use in vivo, in vitro, and ex vivo, respectively.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease; and (3) slowing down disease progression. The term “treating” also encompasses post-treatment care.

In some embodiments, administration of a compound of the invention, or pharmaceutically acceptable salt thereof, is effective in preventing the disease; for example, preventing a disease, condition or disorder in an individual who may be predisposed to the disease, condition or disorder but does not yet experience or display the pathology or symptomatology of the disease.

The compounds of the invention may be used in human and veterinary medicine, which includes the treatment of companion animals, e.g. horses, dogs, cats, rabbits, guinea pigs, fishes e.g. koi, birds e.g. falcon; and livestock, e.g. cattle, poultry, pig, sheep, goat, donkey, yak and camel.

Pharmaceutical Compositions

The present invention further provides pharmaceutical compositions comprising a compound as described herein or a pharmaceutically acceptable salt thereof for use in medicine, e.g. in human or veterinary medicine. In some embodiments, the composition further comprises a pharmaceutically acceptable carrier.

An effective dose of the compounds according to the invention, or their salts, solvates or prodrugs thereof is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are topical or systemical including enteral, oral, rectal, and parenteral, as infusion and injection, intravenous, intra-arterial, intraperitoneal, intramuscular, intracardial, epidural, intracerebral, intracerebroventricular, intraosseous, intra-articular, intraocular, intravitreal, intrathecal, intravaginal, intracavernous, intravesical, subcutaneous, intradermal, transdermal, transmucosal, inhalative, intranasal, buccal, sublingual and intralesional preparations. Particular preference is given to using oral, parenteral, e.g. intravenous or intramuscular, intranasal preparations, e.g. dry powder or sublingual, of the compounds according to the invention. The customary galenic preparation forms, such as tablets, sugar-coated tablets, capsules, dispersible powders, granulates, aqueous solutions, alcohol-containing aqueous solutions, aqueous or oily suspensions, gels, hydrogels, ointments, creams, lotions, shampoos, lip balms, mouthwashs, foams, pastes, tinctures, dermal patches and tapes, forms in occlusion or in combination with time release drug delivery systems, with electrophoretic dermal delivery systems including implants and devices, and with jet injectors, liposome and transfersome vesicles, vapors, sprays, syrups, juices or drops and eye drops, can be used. Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable, synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na-N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy, as sequence therapy or as simultaneous combination therapy, with further active agents, e.g. therapeutically active compounds useful in the treatment of the above indicated disorders.

These therapeutically active compounds may include but are not limited to chemotherapeutic agents such as nucleoside and nucleobase analogs, e.g. Cytarabin, Gemcitabine, Azathioprine, Mercaptopurine, Fluorouracil, Thioguanine, Azacitidine, Capecitabine, Doxifluridine; such as platinum-based drugs, e.g. Cisplatin, Oxaliplatin, Carboplatin and Nedaplatin; such as anthracyclines, e.g. Doxorubicin, Epirubicin, Valrubicin, Idarubicin, Daunorubicin, Sabarubicin, Pixantrone and Mitoxantrone; such as peptide antibiotics, e.g. Actinomycin and Bleomycin; such as alkylating agents e.g. Mechlorethamine, Chlorambucil, Melphalan, Nitrosoureas, Dacarbazine, Temozolomide and Cyclophosphamide; such as antimitotic agents including taxanes and vinca alkaloids, e.g. Docetaxel, Paclitaxel, Abraxane, Cabazitaxel, Vinblastine, Vindesine, Vinorelbine and Vincristine; such as topoisomerase inhibitors, e.g. Irinotecan, Topotecan, Teniposide and Etoposide; such as other cytostatic agents e.g. Hydroxyurea and Methotrexate; such as proteasome inhibitors, e.g Bortezomib, Ixazomib; and other targeted therapeutic agents such as kinase inhibitors, cell cycle inhibitors, regulators i.e. inhibitors and activators of signaling pathways including growth factor signaling, cytokine signaling, NF-kappaB signaling, AP1 signaling, JAK/STAT signaling, EGFR signaling, TGF-beta signaling, Notch signaling, Wnt signaling, Hedgehog signaling, hormone and nuclear receptor signaling, e.g. Erlotinib, Lapatinib, Dasatinib, Imatinib, Afatinib, Vemurafenib, Dabrafenib, Nilotinib, Cetuximab, Trametinib, Palbociclib, Cobimetinib, Cabozantinib, Pegaptanib, Crizotinib, Olaparib, Panitumumab, Cabozantinib, Ponatinib, Regorafenib, Entrectinib, Ranibizumab, Ibrutinib, Trastuzumab, Rituximab, Alemtuzumab, Gefitinib, Bevacizumab, Lenvatinib, Bosutinib, Axitinib, Pazopanib, Everolimus, Temsirolimus, Ruxolitinib, Tofacitinib, Sorafenib, Sunitinib, Aflibercept, Vandetanib; Vismodegib and Sonidegib; retinoids such as retinol, tretinoin, isotretinoin, alitretinoin, bexarotene, tazarotene, acitretin, adapalene and etretinate; hormone signaling modulators including estrogen receptor modulators, androgen receptor modulators and aromatase inhibitors e.g. Raloxifene, Tamoxifen, Fulvestrant, Lasofoxifene, Toremifene, Bicalutamide, Flutamide, Anastrozole, Letrozole and Exemestane; histone deacetylase inhibitors, e.g. Vorinostat, Romidepsin, Panobinostat, Belinostat and Chidamide; and Ingenol mebutate; Valproic acid, Resveratrol, hesperetin, chrysin, phenethyl isothiocyanate, thiocoraline; N-methylhemeanthidine chloride; and immune response modulating agents including immune checkpoint inhibitors e.g. Imiquimod, Ipilimumab, Atezolizumab, Ofatumumab, Rituximab, Nivolumab and Pembrolizumab; and anti-inflammatory agents including glucocorticoids and non-steroidal anti-inflammatory drugs, e.g. cortisol-based preparations, Dexamethason, Betamethason, Prednisone, Prednisolone, Methylprednisolone, Triamcinolon-hexacetonid, Mometasonfuroat, Clobetasolpropionat, acetylsalicylic acid, salicylic acid and other salicylates, Diflunisal, Ibuprofen, Dexibuprofen, Naproxen, Fenoprofen, Ketoprofen, Dexketoprofen, Loxoprofen, Flurbiprofen, Oxaprozin, Indomethacin, Ketorolac, Tolmetin, Diclofenac, Etodolac, Aceclofenac, Nabumetone, Sulindac, Mefenamic acid, Meclofenamic acid, Flufenamic acid, Tolfenamic acid, Celecoxib, Parecoxib, Etoricoxib and Firocoxib; and ACE inhibitors; and beta-blockers; and myostatin inhibitors; and PDE-5 inhibitors; and antihistamines. For a combination therapy, the active ingredients may be formulated as compositions containing several active ingredients in a single dose form and/or as kits containing individual active ingredients in separate dose forms. The active ingredients used in combination therapy may be co-administered or administered separately.

The compounds of the invention may be administered as antibody-drug conjugates.

The compounds of the invention may be administered in combination with surgery, cryotherapy, electrodessication, radiotherapy, photodynamic therapy, laser therapy, chemotherapy, targeted therapy, immunotherapy, gene therapy, antisense therapy, cell-based transplantation therapy, stem cell therapy, physical therapy and occupational therapy.

Chemical Synthesis

### Abbreviations

Ac Acetyl

aq Aqueous

BRSM Based on Recovered Starting Material (yield)

Bu Butyl

DCE 1,2-dichloroethane

DCM Dichloromethane

DIBAL-H Diisobutylaluminium hydride

DMF N,N-dimethylformamide

DMSO Dimethyl sulfoxide

equiv equivalent

ESI Electron Spray Ionization

Et Ethyl

Me Methyl

Ms Methylsulfonyl

mol % mole percent

NMR Nuclear Magnetic Resonance Spectroscopy

PE Petroleum Ether

PTSA p-Toluenesulfonic acid

sat Saturated

TBAF Tetrabutylammonium Fluoride

THF Tetrahydrofuran

TMS Trimethylsilyl

UV Ultraviolet

### General Considerations

The compounds listed in Table 46 and Table 47 have been identified by TLC using pre-coated silica TLC sheets and common organic solvents such as petroleum ether, ethyl acetate, dichloromethane, methanol, toluene, triethylamine or acetic acid as eluent, preferably as binary or tertiary solvent mixtures thereof. UV light at a wavelength of 254 or 366 nm, and/or common staining solutions such as phosphomolybdic acid, potassium permanganate, or ninhydrin were used to visualize the compounds. Reactions were also monitered for completion this way. Reactions were run under inert atmosphere unless otherwise stated. Dry solvents were used wherever required. All reactions were stirred using a stir plate and magnetic stir bar.

The compounds listed in Table 46 have furthermore been identified by mass spectrometry using formic acid in the mobile phase for detection of positive ions, while no additive was used for negative ions. Ammonium Carbonate was used if the molecule was difficult to ionize in negative mode. Representative compounds and those which showed poor ionization in mass spectrometry were also identified by nuclear magnetic resonance spectroscopy (Table 47). Chemical shifts (6) were reported in parts per million (ppm) relative to residual solvent peaks rounded to the nearest 0.01 ppm for proton and 0.1 ppm for carbon (ref.: CHCl₃ [¹H: 7.26 ppm, ¹³C: 77.2 ppm], DMSO [¹H: 2.50 ppm, ¹³C: 39.5 ppm]). Coupling constants (J) were reported in Hz to the nearest 0.1 Hz. Peak multiplicity was indicated as follows: s (singlet), d (doublet), t (triplet), q (quartet), hept (heptet), m (multiplet), and br (broad).

Synthesis of Described Compounds

The aforementioned compounds of the invention falling under the scope of formula I can be synthesized and purified by those persons skilled in the art and are preferably synthesized according to the general procedures (A to I) mentioned herein as illustrated in Scheme 1.

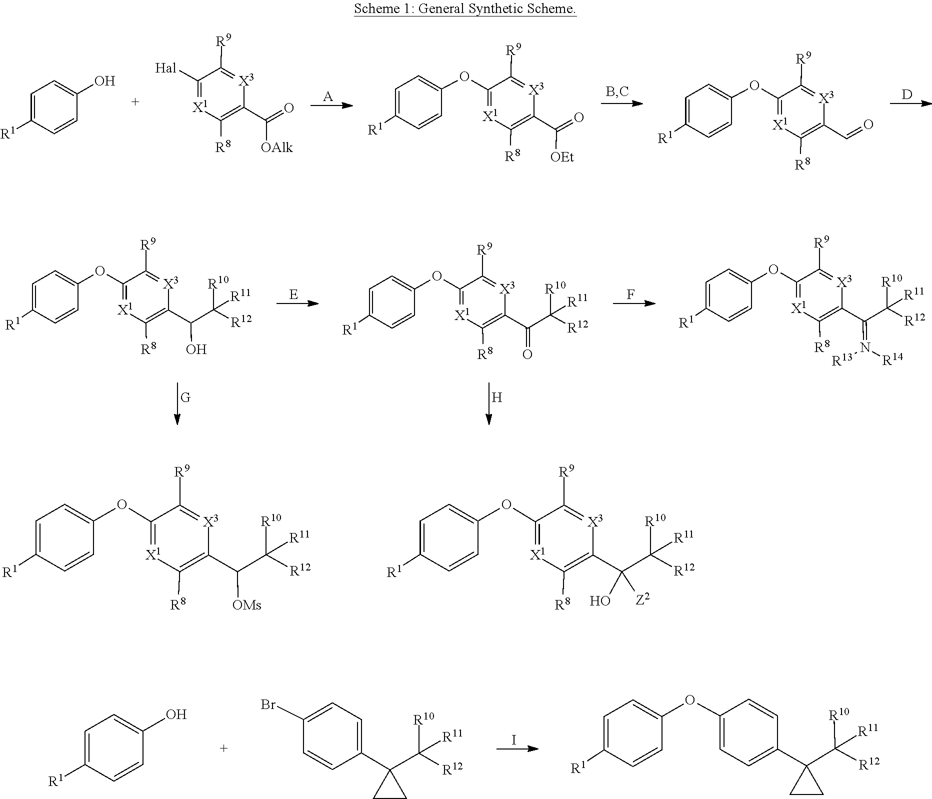

A) To the corresponding mono or bisubstituted phenol (1.0-1.5 equiv) and 4-alkyl ester halo(hetero)aryl (1 equiv), dissolved in DMSO (0.5 M) under argon and stirring, was added K₂CO₃ (1.5 equiv) and the mixture was either stirred at room temperature or heated between 40° C. and 160° C. until full conversion. The mixture was allowed to return to room temperature and was partitioned between an organic solvent, preferably petroleum ether and water. The aqueous layer was extracted twice more and the combined organic phases were then washed with NaOH (aq, 2M) followed by Brine, dried over Na₂SO₄, filtered and concentrated under vacuum. The residue was then purified by flash chromatography (SiO₂, gradient petroleum ether/AcOEt, DCM/MeOH or petroleum ether/AcOEt/NEt₃) to yield the desired bi(hetero)aryl ether ethyl ester.
B) The corresponding bis(hetero)aryl ether alkyl ester (1 equiv) was dissolved in dry THF (0.2 M) under argon and stirring and the resulting solution was cooled to 0° C. with an ice bath. DIBAL-H (2.5 equiv, 1.2 M in toluene) was then added dropwise and the mixture left to stir at that temperature till full conversion. The reaction was quenched via the Fieser method, filtered, concentrated under vacuum and the residue was then purified by flash chromatography (SiO₂, gradient petroleum ether/AcOEt) to yield the desired alcohol.
C) Depending on the scale and substrate, either of these procedures were used.
   To the corresponding alcohol (1 equiv), dissolved in DCM (0.2 M) under vigorous stirring, was added MnO₂ (2-4 equiv). The resulting suspension was stirred at room temperature or 40° C. till full conversion. The reaction was then diluted with AcOEt, filtered over celite and concentrated under vacuum. The residue was then purified by flash chromatography (SiO₂, gradient petroleum ether/AcOEt) to yield the desired aldehyde.
   To the corresponding alcohol (1 equiv), dissolved in DCM or DMSO (0.2 M) under vigorous stirring, was added Dess Martin Periodinane (1.2 equiv). The resulting suspension was stirred at room temperature till full conversion. The solution was diluted in AcOEt and quenched with aq. sat. NaHCO₃ and the phases seperated. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired aldehyde.
   To a solution of oxalyl chloride (2 equiv) in DCM (0.2 M) at −78° C. was added dry DMSO (4 equiv) and the mixture was stirred for 30 min. A solution in DCM (0.2 M) of the corresponding alcohol (1 equiv) was then added followed by freshly distilled NEt₃ (8 equiv). The resulting solution was stirred for 1 hour before being slowly returned to room temperature. The solution was diluted in AcOEt and quenched with aq HCl 1 M and the phases seperated. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired aldehyde.
   In some cases the desired aldehyde proved unstable and was used directly without characterisation in follow-up steps after quick purification using the indicated methods.
D) To the corresponding aldehyde (1 equiv), dissolved in dry THF (0.2 M) at 0° C. under argon and stirring, was added either TMSCF₃ (2 equiv) followed by TBAF (1 mol %) to obtain the corresponding CF₃ bearing secondary alcohol or a Grignard reagent (2 equiv) to obtain the corresponding secondary alkyl alcohol. In both cases, the resulting solution was left to stir at that temperature till full conversion. HCl aq (2.5 M) was then added and the reaction left to stir for a further hour. The reaction was then partitioned between AcOEt and water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired secondary alcohol.
E) To a stirred solution of the corresponding secondary alcohol (1 equiv) in chloroform (0.2 M) at 0° C. was added Dess-Martin Periodinane (1.5 equiv). After completion of the reaction, it was partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired ketone.
F) To a stirred solution of the corresponding ketone (1 equiv) in ethanol or methanol (0.2 M) was added the (hydroxyl)amine (1.2-40 equiv) followed by, either a catalytic amount of PTSA in the case of aliphatic amines, or a base (2.5-40 equiv) in the case of hydroxylamines. The reaction was then refluxed for 24-72 h. After this time, either Celite was added and the volatiles evaporated under vacuum, or the reaction was then partitioned between AcOEt and HCl aq (1 M), the aqueous layer extracted twice more and the combined organic phases washed with Brine, dried over Na₂SO₄, filtered and concentrated under vacuum. In both cases, the residue was then purified by flash chromatography (SiO₂, gradient petroleum ether/AcOEt) to yield the desired imines.
G) To a stirred solution of the corresponding alcohol (1 equiv) in DMF (0.2 M), at 0° C. under argon and stirring, was added trimethylamine (2 equiv) followed by mesyl chloride (1.2 equiv). The reaction was then stirred for 24 h before being partitioned between AcOEt and H₂O. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired mesylate.
H) To the corresponding ketone (1 equiv), dissolved in dry THF (0.2 M) at 0° C. under argon and stirring, was added either TMSCF₃ (1.3 equiv) followed by TBAF (1 mol %) to obtain the corresponding di-CF₃ alcohol or a Grignard reagent (2 equiv) to obtain the corresponding tertiary alcohol. In both cases, the resulting solution was left to stir at that temperature till full conversion. In the first case, after completion, more TBAF (10 mol %) was added followed by water (5.6 equiv) and the reaction left to stir for a further hour. In both cases, the reaction was then partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired tertiary alcohol.
I) To the corresponding 4-substituted phenol (1-2 equiv) and 4-substituted bromoaryl (1-2.5 equiv), dissolved in DMF (0.2 M), was added Cs₂CO₃ (2 equiv), CuI (10 mol %) and tBuXPos (20 mol %). The mixture was degassed using the freeze-pump-thaw method, placed under argon, vigorously stirred and refluxed (165° C.) for 72 h. The mixture was allowed to return to room temperature and was partitioned between petroleum ether and NaOH aq (2 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield the desired bisaryl ether.

Analytical Data

The following compounds were synthetized according to the aforementioned protocols and characterized via mass spectrometry (Table 46) or NMR (Table 47).

TABLE 46

| Compound | | m/z | Ion | m/z | Ion | |
|---|---|---|---|---|---|---|
| No. | Formula | [ESI⁺] | [ESI⁺] | [ESI⁻] | [ESI⁻] | Procedure |
| XPF-0006 | C₂₀H₂₄O₂ | 279.2 | [M − OH]⁺ | | | D |
| XPF-0014 | C₂₄H₂₈O₂ | 331.6 | [M − OH]⁺ | | | D |
| XPF-0042 | C₂₆H₃₀O₂ | 357.3 | [M − OH]⁺ | | | D |
| XPF-0057 | C₁₈H₁₉F₃O₂ | 307.2 | [M − OH]⁺ | | | D |
| XPF-0058 | C₁₈H₁₉F₃O₂ | 307.1 | [M − OH]⁺ | | | D |
| XPF-0062 | C₂₀H₂₁F₃O₂ | 333.2 | [M − OH]⁺ | | | D |
| XPF-0063 | C₁₉H₁₉F₃O₃ | 335.2 | [M − OH]⁺ | | | D |
| XPF-0064 | C₂₀H₂₂F₃NO₂ | 366.3 | [M + H]⁺ | | | D |
| XPF-0065 | C₁₈H₁₈F₃NO₃ | 354.5 | [M + H]⁺ | | | D |
| XPF-0070 | C₂₄H₂₅F₃O₂ | 385.3 | [M − OH]⁺ | | | D |
| XPF-0169 | C₁₉H₂₁F₃O₂ | 321.2 | [M − OH]⁺ | | | H |
| XPF-0170 | C₁₉H₂₁F₃O₂ | 321.1 | [M − OH]⁺ | | | H |
| XPF-0174 | C₂₁H₂₃F₃O₂ | 347.3 | [M − OH]⁺ | | | H |
| XPF-0182 | C₂₅H₂₇F₃O₂ | 399.3 | [M − OH]⁺ | | | H |
| XPF-0202 | C₂₃H₂₅F₃O₂ | 373.3 | [M − OH]⁺ | | | H |
| XPF-0205 | C₂₁H₂₂F₃NO₃ | 394.6 | [M + H]⁺ | | | H |
| XPF-0210 | C₂₇H₂₉F₃O₂ | 425.3 | [M − OH]⁺ | | | H |
| XPF-0230 | C₂₁H₂₀F₆O₂ | | | 417.13 | [M − H]⁻ | H |
| XPF-0258 | C₂₀H₂₂O₂ | 295.2 | [M + H]⁺ | | | E |
| XPF-0266 | C₂₄H₂₆O₂ | 347.6 | [M + H]⁺ | | | E |
| XPF-0421 | C₁₈H₁₇F₃O₂ | | | 321.11 | [M − H]⁻ | E |
| XPF-0422 | C₁₈H₁₇F₃O₂ | | | 321.11 | [M − H]⁻ | E |
| XPF-0426 | C₂₀H₁₉F₃O₂ | | | 374.13 | [M − H]⁻ | E |
| XPF-0429 | C₁₈H₁₆F₃NO₃ | 352.50/370.55 | [M + H]⁺/[M + H₃O]⁺ | | | E |
| XPF-0434 | C₂₄H₂₃F₃O₂ | | | 399.16 | [M − H]⁻ | E |
| XPF-0454 | C₂₁H₂₂F₃NO | 362.3 | [M + H]⁺ | | | F |
| XPF-0469 | C₂₀H₂₀F₃NO₂ | 364.3 | [M + H]⁺ | | | F |
| XPF-0476 | C₂₄H₂₄F₃NO₂ | 416.3 | [M + H]⁺ | | | F |
| XPF-0496 | C₂₁H₂₂F₃NO₂ | 378.3 | [M + H]⁺ | | | F |
| XPF-0518 | C₂₆H₂₇F₃O | | | 411.2 | [M − H]⁻ | 1 |
| XPF-0630 | C₂₄H₂₂₄F₄O₂ | 404.3 | [M − OH]⁺ | | | D |
| XPF-1162 | C₂₅H₂₉NO₂ | 376.4 | [M + H]⁺ | | | D |
| XPF-1178 | C₁₇H₁₈F₃NO₂ | 326.2 | [M + H]⁺ | | | D |
| XPF-1182 | C₁₉H₂₀F₃NO₂ | 352.3 | [M + H]⁺ | 350.32 | [M − H]⁻ | D |
| XPF-1185 | C₁₇H₁₇F₃N₂O₃ | 355.5 | [M + H]⁺ | | | D |
| XPF-1190 | C₂₃H₂₄F₃NO₂ | 404.3 | [M + H]⁺ | | | D |
| XPF-1196 | C₂₀H₂₂F₃NO₄S | 430.2 | [M + H]⁺ | | | G |
| XPF-1322 | C₂₂H₂₄F₃NO₂ | 392.3 | [M + H]⁺ | | | H |
| XPF-1325 | C₂₀H₂₁F₃N₂O₃ | 395.6 | [M + H]⁺ | | | H |
| XPF-1330 | C₂₆H₂₈F₃NO₂ | 444.3 | [M + H]⁺ | | | H |
| XPF-1541 | C₁₇H₁₆F₃NO₂ | 324.17/342.19 | [M + H]⁺/[M + H₃O]⁺ | | | E |
| XPF-1542 | C₁₇H₁₆F₃NO₂ | 342.19/324.16 | [M + H]⁺/[M + H₃O]⁺ | | | E |
| XPF-1546 | C₁₉H₁₈F₃NO₂ | 350.22/368.24 | [M + H]⁺/[M + H₃O]⁺ | | | E |
| XPF-1549 | C₁₇H₁₅F₃N₂O₃ | 353.50/371.56 | [M + H]⁺/[M + H₃O]⁺ | | | E |
| XPF-1554 | C₂₃H₂₂F₃NO₂ | 402.29/420.3 | [M + H]⁺/[M + H₃O]⁺ | | | E |
| XPF-1588 | C₁₉H₁₉F₃N₂O₂ | 365.3 | [M + H]⁺ | 363.33 | [M − H]⁻ | F |
| XPF-1596 | C₂₃H₂₃F₃N₂O₂ | 417.3 | [M + H]⁺ | 415.31 | [M − H]⁻ | F |
| XPF-1602 | C₂₀H₂₁F₃N₂O₂ | 379.3 | [M + H]⁺ | | | F |
| XPF-1616 | C₂₀H₂₁F₃N₂O₂ | 379.3 | [M + H]⁺ | | | F |
| XPF-1624 | C₂₄H₂₅F₃N₂O₂ | 431.3 | [M + H]⁺ | | | F |
| XPF-2248 | C₂₁H₂₃F₃O₂ | 347.5 | [M − OH]⁺ | | | D |
| XPF-2249 | C₂₁H₂₂F₃NO₂ | | | 376.58 | [M − H]⁻ | F |
| XPF-2251 | C₂₂H₂₃F₃N₂O₂ | 405.6 | [M + H]⁺ | | | C |
| XPF-2252 | C₂₄H₂₆F₃NO₂ | 418.7 | [M + H]⁺ | | | C |
| XPF-2253 | C₂₄H₂₄F₃NO₂ | 416.66/434.66 | [M + H]⁺/[M + H₃O]⁺ | | | E |
| XPF-2254 | C₂₂H₂₁F₃N₂O₂ | 421.7 | [M + H₃0]⁺ | | | E |
| XPF-I-0001 | C₁₉H₂₂O₃ | 299.2 | [M + H]⁺ | | | A |
| XPF-I-0002 | C₂₁H₂₄O₃ | 325.3 | [M + H]⁺ | | | A |
| XPF-I-0003 | C₁₉H₂₂O₃ | 299.2 | [M + H]⁺ | | | A |
| XPF-I-0004 | C₂₅H₂₈O₃ | 377.4 | [M + H]⁺ | | | A |
| XPF-I-0005 | C₂₀H₂₂O₄ | 327.2 | [M + H]⁺ | | | A |
| XPF-I-0006 | C₂₁H₂₅NO₃ | 340.4 | [M + H]⁺ | | | A |
| XPF-I-0007 | C₂₅H₂₇FO₃ | 395.3 | [M + H]⁺ | | | A |
| XPF-I-0008 | C₂₀H₂₃NO₃ | 326.3 | [M + H]⁺ | | | A |
| XPF-I-0009 | C₁₈H₂₁NO₃ | 300.2 | [M + H]⁺ | | | A |
| XPF-I-0010 | C₁₈H₂₁NO₃ | 300.2 | [M + H]⁺ | | | A |
| XPF-I-0011 | C₂₄H₂₇NO₃ | 378.3 | [M + H]⁺ | | | A |
| XPF-I-0012 | C₁₉H₂₂O₂ | 265.1 | [M − OH]⁺ | | | B |
| XPF-I-0013 | C₁₈H₂₁NO₂ | 284.1 | [M + H]⁺ | | | B |
| XPF-I-0014 | C₁₇H₂₀O₂ | 239.2 | [M − OH]⁺ | | | B |
| XPF-I-0015 | C₁₇H₂₀O₂ | 239.2 | [M − OH]⁺ | | | B |
| XPF-I-0016 | C₂₃H₂₆O₂ | 317.2 | [M − OH]⁺ | | | B |
| XPF-I-0017 | C₁₆H₁₉NO₂ | 258.1 | [M + H]⁺ | | | B |
| XPF-I-0018 | C₁₆H₁₉NO₂ | 258.1 | [M + H]⁺ | | | B |
| XPF-I-0019 | C₂₂H₂₅NO₂ | 336.3 | [M + H]⁺ | | | B |
| XPF-I-0020 | C₁₈H₂₀O₃ | 267.1 | [M − OH]⁺ | | | B |
| XPF-I-0021 | C₁₉H₂₃NO₂ | 298.2 | [M + H]⁺ | | | B |
| XPF-I-0022 | C₂₃H₂₅FO₂ | 335.2 | [M − OH]⁺ | | | B |
| XPF-I-0023 | C₁₉H₂₀O₂ | 281.2 | [M + H]⁺ | | | C |
| XPF-I-0024 | C₁₈H₁₉NO₂ | 282.1 | [M + H]⁺ | | | C |
| XPF-I-0025 | C₁₇H₁₈O₂ | 255.1 | [M + H]⁺ | | | C |
| XPF-I-0026 | C₁₇H₁₈O₂ | 255.1 | [M + H]⁺ | | | C |
| XPF-I-0027 | C₂₃H₂₄O₂ | 333.3 | [M + H]⁺ | | | C |
| XPF-I-0028 | C₁₆H₁₇NO₂ | 256.1 | [M + H]⁺ | | | C |
| XPF-I-0029 | C₁₆H₁₇NO₂ | 256.1 | [M + H]⁺ | | | C |
| XPF-I-0030 | C₂₂H₂₃NO₂ | 334.3 | [M + H]⁺ | | | C |
| XPF-I-0031 | C₁₈H₁₈O₃ | 283.1 | [M + H]⁺ | | | C |
| XPF-I-0032 | C₁₈H₂₁NO₂ | 296.2 | [M + H]⁺ | | | C |
| XPF-I-0033 | C₂₃H₂₃FO₂ | 351.3 | [M + H]⁺ | | | C |
| XPF-I-0035 | C₂₀H₂₄O₂ | 296.5 | [M + H]⁺ | | | B |
| XPF-I-0037 | C₂₁H₂₃ClO₃ | 359.6 | [M + H]⁺ | | | A |
| XPF-I-0038 | C₂₅H₂₇ClO₃ | 411.6 | [M + H]⁺ | | | A |
| XPF-I-0039 | C₂₁H₂₃BrO₃ | 403.56/405.55 | [M + H]⁺ | | | A |
| XPF-I-0041 | C₁₉H₂₁NO₄ | 328.5 | [M + H]⁺ | | | A |
| XPF-I-0042 | C₁₈H₂₀N₂O₄ | 329.5 | [M + H]⁺ | | | A |
| XPF-I-0043 | C₁₇H₁₉NO₃ | 286.4 | [M + H]⁺ | | | B |
| XPF-I-0044 | C₁₉H₂₁ClO₂ | 299.4 | [M − OH]⁺ | | | B |
| XPF-I-0045 | C₂₃H₂₅ClO₂ | 351.6 | [M − OH]⁺ | | | B |
| XPF-I-0046 | C₁₉H₂₁BrO₂ | 343.45/354.43 | [M − OH]⁺ | | | B |
| XPF-I-0047 | C₂₃H₂₅BrO₂ | 395.56/397.55 | [M − OH]⁺ | | | B |
| XPF-I-0052 | C₂₂H₂₄N₂O₃ | 365.6 | [M + H]⁺ | | | A |
| XPF-I-0053 | C₂₁H₂₄N₂O₂ | 337.6 | [M + H]⁺ | | | B |
| XPF-I-0054 | C₂₃H₂₇NO₂ | 350.6 | [M + H]⁺ | | | B |
| XPF-I-0055 | C₂₄H₂₇NO₃ | 378.6 | [M + H]⁺ | | | A |
| XPF-I-0056 | C₂₁H₂₂N₂O₂ | 335.6 | [M + H]⁺ | | | C |
| XPF-I-0057 | C₂₃H₂₅NO₂ | 348.6 | [M + H]⁺ | | | C |
| XPF-I-0058 | C₁₆H₁₈N₂O₃ | 287.4 | [M + H]⁺ | | | B |

TABLE 47

| Com- | | | Pro- |
|---|---|---|---|
| pound | | | ce- |
| No. | Formula | ¹H-NMR | dure |
| XPF- 0504 | C₂₅H₂₆F₃NO₂ | ¹H NMR (300 MHz, DMSO) δ 7.56- 7.36 (m, 4H), 7.10-7.00 (m, 4H), | F |
| | | 3.33 (s, 3H), 2.07 (s, 3H), 1.88 (d, J = 3.0 Hz, 6H), 1.75 (t, J = 3.1 Hz, | |
| | | 6H). | |
| XPF- 2241 | C₂₀H₂₀ClF₃O₂ | ¹H NMR (400 MHz, CDCl₃) δ 7.35 (d, J = 8.6 Hz, 2H), 7.24 (d, J = 2.2 | D |
| | | Hz, 1H), 7.02 (dd, J = 8.4, 2.1 Hz, 1H), 6.92-6.85 (m, 3H), 4.93 (q, J = | |
| | | 6.7 Hz, 1H), 2.43 (s, 1H), 1.89-1.64 (m, 5H), 1.38-1.12 (m, 5H). | |
| XPF- 2242 | C₂₄H₂₄ClF₃O₂ | ¹H NMR (400 MHz, CDCl₃) δ 7.46- 7.38 (m, 3H), 7.24 (dd, J = 8.6, 2.3 | D |
| | | Hz, 1H), 7.00 (d, J = 8.6 Hz, 1H), 6.98-6.92 (m, 2H), 5.00 (q, J = 6.7 Hz, | |
| | | 1H), 2.50 (s, 1H), 2.12 (s, 3H), 1.90 (d, J = 2.9 Hz, 6H), 1.85-1.69 (m, | |
| | | 6H). | |
| XPF- 2243 | C₂₀H₂₀BrF₃O₂ | ¹H NMR (400 MHz, CDCl₃) δ 7.48 (d, J = 2.1 Hz, 1H), 7.42 (d, J = 8.5 | D |
| | | Hz, 2H), 7.13 (dd, J = 8.3, 2.2 Hz, 1H), 6.98-6.92 (m, 3H), 5.00 (q, J = | |
| | | 6.7 Hz, 1H), 2.49 (s, 1H), 1.93-1.69 (m, 5H), 1.47-1.18 (m, 5H). | |
| XPF- 2244 | C₂₄H₂₄BrF₃O₂ | ¹H NMR (400 MHz, CDCl₃) δ 7.55-7.51 (m, 1H), 7.35 (d, J = 8.5 Hz, | D |
| | | 2H), 7.25-7.17 (m, 1H), 6.90 (dd, J = 8.7, 6.8 Hz, 3H), 4.93 (q, J = 6.8 | |
| | | Hz, 1H), 2.04 (s, 3H), 1.88-1.79 (m, 6H), 1.70 (q, J = 12.4 Hz, 6H). | |
| XPF- 2245 | C₂₄H₂₂ClF₃O₂ | ¹H NMR (400 MHz, CDCl₃) δ 8.08-8.02 (m, 2H), 7.47 (d, J = 2.3 Hz, | E |
| | | 1H), 7.31 (dd, J = 8.5, 2.3 Hz, 1H), 7.10 (d, J = 8.5 Hz, 1H), 7.00-6.94 | |
| | | (m, 2H), 2.13 (s, 3H), 1.92 (d, J = 2.9 Hz, 6H), 1.79 (q, J = 12.5 Hz, 6H). | |
| XPF- 2246 | C₂₀H₁₈BrF₃O₂ | ¹H NMR (400 MHz, CDCl₃) δ 8.05 (d, J = 8.3 Hz, 2H), 7.51 (d, J = 2.1 | E |
| | | Hz, 1H), 7.20 (dd, J = 8.3, 2.1 Hz, 1H), 7.06 (d, J = 8.3 Hz, 1H), 7.01- | |
| | | 6.95 (m, 2H), 2.53 (s, 1H), 1.95-1.72 (m, 5H), 1.48-1.18 (m, 5H). | |
| XPF- 2247 | C₂₄H₂₂BrF₃O₂ | ¹H NMR (400 MHz, CDCl₃) δ 8.10-7.99 (m, 2H), 7.63 (d, J = 2.3 Hz, | E |
| | | 1H), 7.36 (dd, J = 8.5, 2.3 Hz, 1H), 7.08 (d, J = 8.5 Hz, 1H), 7.02-6.94 | |
| | | (m, 2H), 2.13 (s, 3H), 1.92 (d, J = 2.9 Hz, 6H), 1.79 (q, J = 12.7 Hz, 6H). | |
| XPF- 2250 | C₂₁H₂₁F₃O₂ | ¹H NMR (400 MHz, CDCl₃) δ 8.00-7.92 (m, 2H), 7.06 (d, J = 1.8 Hz, | E |
| | | 1H), 7.01 (dd, J = 8.2, 2.3 Hz, 1H), 6.86 (t, J =8.8 Hz, 3H), 2.50-2.36 | |
| | | (m, 1H), 2.07 (s, 3H), 1.88-1.65 (m, 5H), 1.43-1.12 (m, 5H). | |
| XPF-I- 0034 | C₂₂H₂₆O₃ | ¹H NMR (400 MHz, CDCl₃) δ 8.00-7.92 (m, 2H), 7.10 (d, J = 2.2 Hz, | A |
| | | 1H), 7.04 (dd, J = 8.3, 2.3 Hz, 1H), 6.91-6.83 (m, 3H), 4.35 (q, J = 7.1 | |
| | | Hz, 2H), 2.54-2.40 (m, 1H), 2.15 (s, 3H), 1.95-1.70 (m, 5H), 1.46- | |
| | | 1.18 (m, 8H). | |
| XPF-I- 0036 | C₂₀H₂₂O₂ | ¹H NMR (400 MHz, CDCl₃) δ 9.90 (s, 1H), 7.84-7.78 (m, 2H), 7.12 (d, J = | C |
| | | 2.2 Hz, 1H), 7.09-7.04 (m, 1H), 6.98- 6.93 (m, 2H), 6.92 (d, J = 8.2 | |
| | | Hz, 1H), 2.55-2.41 (m, 1H), 2.15 (s, 3H), 1.95-1.70 (m, 5H), 1.50- | |
| | | 1.17 (m, 5H). | |
| XPF-I- 0040 | C₂₅H₂₇BrO₃ | ¹H NMR (400 MHz, CDCl₃) δ 8.05-7.95 (m, 2H), 7.61 (d, J = 2.3 Hz, | A |
| | | 1H), 7.31 (dd, J = 8.5, 2.3 Hz, 1H), 7.02 (d, J = 8.5 Hz, 1H), 6.95-6.87 | |
| | | (m, 2H), 4.35 (q, J = 7.1 Hz, 2H), 2.12 (s, 3H), 1.91 (d, J = 2.9 Hz, 6H), | |
| | | 1.78 (q, J = 12.5 Hz, 6H), 1.38 (d, J = 7.1 Hz, 2H). | |
| XPF-I- 0048 | C₁₉H₁₉ClO₂ | ¹H NMR (400 MHz, CDCl₃) δ 9.85 (s, 1H), 7.81-7.73 (m, 2H), 7.28 (dd, | C |
| | | J = 9.8, 2.2 Hz, 1H), 7.10-7.04 (m, 1H), 6.99 (d, J = 8.3 Hz, 1H), 6.92 | |
| | | (dd, J = 6.8,1.9 Hz, 2H), 2.45 (s, 1H), 1.89-1.64 (m, 6H), 1.37-1.11 | |
| | | (m, 6H). | |
| XPF-I- 0049 | C₂₃H₂₃ClO₂ | ¹H NMR (400 MHz, CDCl₃) δ 9.92 (s, 1H), 7.88-7.80 (m, 2H), 7.46 (d, J = | C |
| | | 2.3 Hz, 1H), 7.30 (dd, J = 8.6, 2.3 Hz, 1H), 7.08 (d, J = 8.5 Hz, 1H), | |
| | | 7.02-6.94 (m, 2H), 2.13 (s, 3H), 1.92 (d, J = 2.9 Hz, 6H), 1.78 (q, J = | |
| | | 12.6 Hz, 6H). | |
| XPF-I- 0050 | C₁₉H₁₉BrO₂ | ¹H NMR (400 MHz, CDCl₃) δ 9.92 (s, 1H), 7.89-7.80 (m, 2H), 7.50 (d, J = | C |
| | | 2.1 Hz, 1H), 7.19 (dd, J = 8.3, 2.1 Hz, 1H), 7.04 (d, J = 8.3 Hz, 1H), | |
| | | 7.01-6.95 (m, 2H), 2.52 (s, 1H), 1.99-1.70 (m, 5H), 1.48-1.17 (m, | |
| | | 5H). | |
| XPF-I- 0051 | C₂₃H₂₃BrO₂ | ¹H NMR (400 MHz, CDCl₃) δ 9.92 (s, 1H), 7.88-7.81 (m, 2H), 7.62 (d, J = | C |
| | | 2.3 Hz, 1H), 7.34 (dd, J = 8.5, 2.3 Hz, 1H), 7.06 (d, J = 8.5 Hz, 1H), | |
| | | 7.02-6.97 (m, 2H), 2.13 (s, 3H), 1.91 (d, J = 2.9 Hz, 6H), 1.78 (q, J = | |
| | | 12.7 Hz, 6H). | |

For illustrative purposes the synthesis and characterisation of the following examples are described in detail.

### XPF-0062: 1-(4-(4-cyclohexylphenoxy)phenyl)-2,2,2-trifluoroethan-1-ol

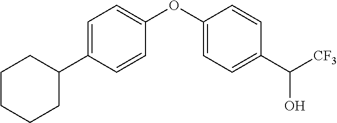 [SMILES: OC(c1ccc(Oc2ccc(C3CCCCC3)cc2)cc1)C(F)(F)F]

To 4-(4-cyclohexylphenoxy)benzaldehyde (1.84 g, 6.55 mmol, 1 equiv), dissolved in dry THF (26.2 mL, 0.2 M) at 0° C. under argon and stirring, was added TMSCF₃ (1.93 mL, 13.1 mmol, 2 equiv) followed by TBAF (65 μL, 66 μmol, 1 mol %). The resulting solution was left to stir at that temperature till full conversion. HCl aq (2.5 M) was then added and the reaction left to stir for a further hour. The reaction was then partitioned between AcOEt and water. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 2.13 g of 1-(4-(4-cyclohexylphenoxy)phenyl)-2,2,2-trifluoroethan-1-ol (93%).

MS: m/z [M-OH]⁺, calc for [C₂₀H₂₀F₃O]⁺=333.14; found 333.19

¹H-NMR (300 MHz, CDCl₃) δ 7.41 (dt, J=9.0, 0.6 Hz, 2H), 7.23-7.16 (m, 2H), 7.04-6.91 (m, 4H), 5.00 (qd, J=6.7, 4.4 Hz, 1H), 2.61-2.37 (m, 2H), 1.99-1.67 (m, 5H), 1.50-1.19 (m, 5H).

¹³C-NMR (75 MHz, CDCl3) δ 159.1, 154.2, 143.9, 128.9, 128.1, 128.0, 124.3 (q, J=282.0 Hz), 119.4, 118.1, 72.47 (q, J=32.2 Hz), 43.9, 34.6, 26.9, 26.1.

### XPF-0434: 1-(4-(4-(adamantan-1-yl)phenoxy)phenyl)-2,2,2-trifluoroethan-1-one

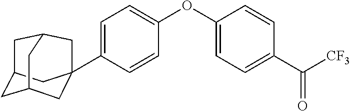 [SMILES: O=C(c1ccc(Oc2ccc(C34CC5CC(CC(C5)C3)C4)cc2)cc1)C(F)(F)F]

To a stirred solution of 1-(4-(4-(adamantan-1-yl)phenoxy)phenyl)-2,2,2-trifluoroethan-1-ol (750 mg, 1.86 mmol, 1 equiv) in chloroform (9.3 mL, 0.2 M) at 0° C. was added Dess-Martin Periodinane (1.03 g, 2.42 mmol, 1.5 equiv). After completion of the reaction, it was partitioned between AcOEt and NaHCO₃ aq sat. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 647 mg of 1-(4-(4-(adamantan-1-yl)phenoxy)phenyl)-2,2,2-trifluoroethan-1-one (87%).

MS: m/z [M+H]⁺, calc for [C₂₄H₂₁F₃O₂]⁺=399.16; found 399.16

¹H-NMR (300 MHz, CDCl₃) δ 8.10-7.89 (m, 2H), 7.44-7.25 (m, 2H), 7.02-6.86 (m, 4H), 2.05 (p, J=3.1 Hz, 3H), 1.86 (d, J=2.9 Hz, 6H), 1.80-1.60 (m, 6H).

¹³C-NMR (75 MHz, CDCl₃) δ 179.1 (q, J=31 Hz), 164.6, 152.0, 148.7, 132.7 (q, J=2.3 Hz), 126.7, 123.9 (q, J=291 Hz), 120.2, 117.1, 43.3, 36.7, 36.1, 28.9.

### XPF-1330: 1-(6-(4-(adamantan-1-yl)phenoxy)pyridin-3-yl)-1-cyclopropyl-2,2,2-trifluoroethan-1-ol

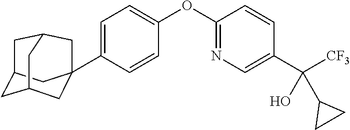 [SMILES: OC(c1ccc(Oc2ccc(C34CC5CC(CC(C5)C3)C4)cc2)nc1)(C1CC1)C(F)(F)F]

To 1-(6-(4-(adamantan-1-yl)phenoxy)pyridin-3-yl)-2,2,2-trifluoroethan-1-one (52 mg, 0.13 mmol, 1 equiv), dissolved in dry THF (0.8 mL, 0.16M) at 0° C. under argon and stirring, was added cyclopropyl magnesium bromide (0.6 mL, 0.26 mmol, 2 equiv, 0.4 M solution in THF). The resulting solution was left to stir at that temperature till full conversion. After completion, the reaction was partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 43 mg of 1-(6-(4-(adamantan-1-yl)phenoxy)pyridin-3-yl)-1-cyclopropyl-2,2,2-trifluoroethan-1-ol (75%).

MS: m/z [M+H]⁺, calc for [C₂₆H₂₉F₃NO₂]⁺₌444.21; found 444.30

¹H-NMR (300 MHz, CDCl₃) δ 8.38 (d, J=2.5 Hz, 1H), 8.05 (dd, J=8.7, 2.6 Hz, 1H), 7.45-7.31 (m, 2H), 7.17-6.96 (m, 3H), 6.23 (s, 1H), 2.08 (q, J=3.1 Hz, 3H), 1.89 (d, J=3.0 Hz, 6H), 1.79-1.62 (m, 7H), 0.87-0.72 (m, 1H), 0.64-0.48 (m, 1H), 0.40 (tdd, J=9.1, 5.9, 4.1 Hz, 1H), 0.27 (dtd, J=9.5, 5.9, 4.2 Hz, 1H).

¹³C-NMR (300 MHz, CDCl₃) δ 163.6, 151.7, 147.7, 146.5, 139.3, 130.4, 126.3, 121.3, 110.7, 73.68 (d, J=27.6 Hz), 43.1, 36.5, 35.9, 28.7, 14.8, 1.6. (one remaining CF₃ group not visible due to relaxation times)

### XPF-2249: 1-(4-(4-cyclohexyl-2-methylphenoxy)phenyl)-2,2,2-trifluoroethan-1-one oxime

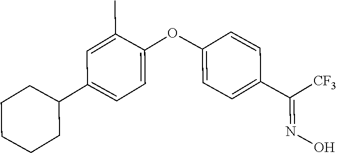 [SMILES: Cc1cc(C2CCCCC2)ccc1Oc1ccc(/C(=N/O)C(F)(F)F)cc1]

To a stirred solution of 1-(4-(4-cyclohexyl-2-methylphenoxy)phenyl)-2,2,2-trifluoroethan-1-one (50 mg, 0.14 mmol, 1 equiv) in methanol (0.7 mL, 0.2 M) was added hydroxylamine hydrochloride (11.5 mg, 0.17 mmol, 1.2 equiv) followed by sodium acetate (34 mg, 0.41 mmol, 3 equiv). The reaction was then refluxed for 24 h before being partitioned between AcOEt and HCl aq (1 M). The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 38 mg of 1-(4-(4-cyclohexyl-2-methylphenoxy)phenyl)-2,2,2-trifluoroethan-1-one oxime (73%).

MS: m/z [M−H]⁻, calc for [C₂₁H₂₁F₃NO₂]⁺=376.15; found 376.58

¹H NMR (400 MHz, CDCl₃) δ 8.56 (brs, 0.3H), 8.54 (s, 0.7H), 7.56-7.47 (m, 1.5H), 7.45-7.38 (m, 0.5H), 7.15-7.06 (m, 1H), 7.07-7.01 (m, 1H), 6.98-6.85 (m, 3H), 2.49 (tt, J=11.5, 3.8 Hz, 1H), 2.18 (s, 2H), 2.17 (s, 1H), 1.96-1.80 (m, 4H), 1.80-1.72 (m, 1H), 1.49-1.33 (m, 4H), 1.33-1.19 (m, 1H).

¹³C NMR (101 MHz, CDCl₃) δ 160.33, 160.22, 151.08, 150.93, 145.01, 144.93, 130.59, 130.07, 130.01, 125.62, 120.71, 120.58, 118.96, 116.36, 116.22, 44.02, 34.63, 26.92, 26.16, 16.22.

### XPF-0518: 1-(4-(4-(1-(trifluoromethyl)cyclopropyl)-phenoxy)phenyl)adamantine

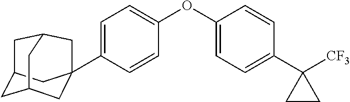 [SMILES: FC(F)(F)C1(c2ccc(Oc3ccc(C45CC6CC(CC(C6)C4)C5)cc3)cc2)CC1]

To 4-(adamantan-1-yl)phenol (137 mg, 0.6 mmol, 1.5 equiv) and 1-bromo-4-(1-(trifluoromethyl)cyclopropyl)benzene (106 mg, 0.4 mmol, 1 equiv), dissolved in DMF (1.6 mL, 0.2 M), was added Cs₂CO₃ (260 mg, 0.8 mmol, 2 equiv), CuI (7.6 mg, 40 μmol, 10 mol %) and tBuXPos (34 mg, 80 μmol, 20 mol %). The mixture was degassed using the freeze, pump, thaw method, placed under argon, vigorously stirred and refluxed (165° C.) for 72 h. The mixture was allowed to return to room temperature and was partitioned between petroleum ether and NaOH aq. 2 M. The aqueous layer was extracted twice more and the combined organic phases were then washed with Brine, dried over Na₂SO₄, filtered and concentrated under vacuum. The residue was then purified by flash chromatography (SiO₂, gradient petroleum ether/AcOEt) to yield 120 mg of 1-(4-(4-(1-(trifluoromethyl)cyclopropyl)-phenoxy)phenyl)adamantine (72%).

MS: calc for [C₂₆H₂₆F₃O]⁺=411.19; found 411.20

¹H-NMR (300 MHz, CDCl₃) δ 7.43-7.36 (m, 2H), 7.36-7.30 (m, 2H), 7.03-6.91 (m, 4H), 2.18-2.04 (m, 3H), 1.95-1.88 (m, 6H), 1.86-1.68 (m, 6H), 1.38-1.30 (m, 2H), 1.05-0.97 (m, 2H).

¹³C-NMR (300 MHz, CDCl3) δ 157.9, 154.2, 146.9, 132.6, 130.3, 126.2, 126.42 (q, J=273.5 Hz) 118.9, 118.0, 43.3, 36.8, 35.9, 27.5 (q, J=33.3 Hz), 9.81 (q, J=2.3 Hz).

A compound according to general formula (I) as defined herein or a salt or solvate thereof:

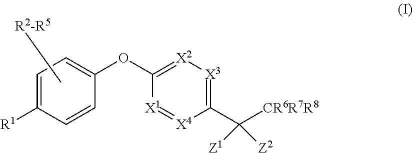

R¹=C₁-C₁₂ preferably C₄-C₁₂ alkyl, C₂-C₁₂ preferably C₄-C₁₂ alkenyl, C₂-C₁₂ preferably C₄-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, —OC₁-C₁₂ preferably —OC₃-C₁₂ alkyl, —OC₂-C₁₂ preferably —OC₃-C₁₂ alkenyl, —OC₂-C₁₂ preferably —OC₃-C₁₂ alkynyl, —OC₃-C₈ cycloalkyl, —OC₅-C₈ cycloalkenyl, —OC₅-C₁₂ bicycloalkyl, —OC₇-C₁₂ bicycloalkenyl, —OC₈-C₁₄ tricycloalkyl, —SC₁-C₁₂ preferably —SC₃-C₁₂ alkyl, —SC₂-C₁₂ preferably —SC₃-C₁₂ alkenyl, —SC₂-C₁₂ preferably —SC₃-C₁₂ alkynyl, —SC₃-C₈ cycloalkyl, —SC₅-C₈ cycloalkenyl, —SC₅-C₁₂ bicycloalkyl, —SC₇-C₁₂ bicycloalkenyl, —SC₈-C₁₄ tricycloalkyl, —NHR⁹ or —NR⁹R¹⁰ wherein R⁹ and R¹⁰ are independently from each other selected from: C₁-C₁₂ preferably C₃-C₁₂ alkyl, C₂-C₁₂ preferably C₃-C₁₂ alkenyl, C₂-C₁₂ preferably C₃-C₁₂ alkynyl, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, or wherein R⁹ can form a ring structure together with R¹⁰ wherein the said ring structure including the N-atom is selected from three to eight membered cyclic structures or five to twelve membered bicyclic structures and wherein all said ring structures can additionally contain one or more heteroatoms independently selected from 0, S and N in replacement of a carbon atom contained in the ring structure, and particularly wherein such a replacement results in residues that contain at least twice the number of C atoms than heteroatoms independently selected from O, S and N;

wherein all alkyl, alkenyl and alkynyl residues contained in the definitions of R¹, R⁹ and R¹⁰ are linear or branched, and are unsubstituted or substituted with one or more substituents independently selected from: —F, —Cl, —Br, —I, —CN, —NCO, —NCS, —OH, —NH₂, —NO₂, ═O, C₃-C₈ cycloalkyl, C₅-C₈ cycloalkenyl, C₅-C₁₂ bicycloalkyl, C₇-C₁₂ bicycloalkenyl, C₈-C₁₄ tricycloalkyl, linear or branched —OC₁-C₅ alkyl such as —OCH₃, —OC₃-C₈ cycloalkyl such as —O(cyclopropyl), linear or branched —NH(C₁-C₅ alkyl), linear or branched —N(C₁-C₅ alkyl)(C₁-C₅ alkyl), —NH(C₃-C₈ cycloalkyl) such as —NH(cyclopropyl), —N(C₃-C₈ cycloalkyl)(C₃-C₈ cycloalkyl), linear or branched —N(C₁-C₅ alkyl)(C₃-C₈ cycloalkyl);

wherein when an alkyl, alkenyl and alkynyl residue contained in the definitions of R¹, R⁹ and R¹⁰ is substituted with one or more substituents being ═O, such substitution with ═O cannot result in one of the groups selected from C═O, S═O and N═O directly bound to an aromatic ring;

wherein all cyclic structures, bicyclic structures and tricyclic structures including cycloalkyl, cycloalkenyl, bicycloalkyl, bicycloalkenyl and tricycloalkyl residues contained in the definitions of R¹, R⁹ and R¹⁰ are unsubstituted or substituted with one or more substituents independently selected from: —F, —Cl, —Br, —I, —CN, —NCO, —NCS, —OH, —NH₂, —NO₂, ═O, linear or branched C₁-C₅ alkyl such as —CH₃, linear or branched —OC₁-C₅ alkyl such as —OCH₃, linear or branched —NH(C₁-C₅ alkyl), linear or branched —N(C₁-C₅ alkyl)(C₁-C₅ alkyl), —NH(C₃-C₈ cycloalkyl) such as —NH(cyclopropyl), —N(C₃-C₈ cycloalkyl)(C₃-C₈ cycloalkyl), linear or branched —N(C₁-C₅ alkyl)(C₃-C₈ cycloalkyl);

wherein all alkyl, alkenyl and alkynyl residues contained in the definitions of R¹, R⁹ and R¹⁰ can contain one or more heteroatoms independently selected from O, S and N in replacement of a carbon atom, and wherein such a replacement results in residues that contain at least twice the number of C atoms than heteroatoms independently selected from O, S and N, and wherein such replacement additionally cannot result in one of the groups selected from C═O, S═O and N═O directly bound to an aromatic ring;

wherein all cycloalkyl, cycloalkenyl, bicycloalkyl, bicycloalkenyl and tricycloalkyl residues contained in the definitions of R¹, R⁹ and R¹⁰ can contain one or more heteroatoms independently selected from O, S and N in replacement of a carbon atom, and wherein such a replacement results in residues that contain at least the same number of C atoms than heteroatoms independently selected from O, S and N;

wherein all alkyl, alkenyl, alkynyl, cycloalkyl, cycloalkenyl, bicycloalkyl, bicycloalkenyl and tricycloalkyl residues contained in the definitions of R¹, R⁹ and R¹⁰ can be partially or fully halogenated, particularly fluorinated, more particularly perfluorinated;

wherein bicyclic and tricyclic residues include fused, bridged and spiro systems; R²-R⁵ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl and cycloalkyl residues contained in the definitions of R²-R⁵ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂; wherein all alkyl, alkenyl, alkynyl and cycloalkyl residues contained in the definitions of R²-R⁵ can contain one or more heteroatoms independently selected from O, S and N in replacement of a carbon atom, and wherein such replacement cannot result in one of the groups selected from C═O and S═O directly bound to an aromatic ring;

X¹-X⁴ are independently from each other selected from N, CR¹¹, CR¹², CR¹³, CR¹⁴;

R¹¹-R¹⁴ are independently from each other selected from —H, —F, —Cl, —Br, —I, —CN, —NCO, —NCS, —OH, —NH₂, —NO₂, linear or branched C₁-C₄ alkyl, linear or branched C₂-C₄ alkenyl, linear or branched C₂-C₄ alkynyl, C₃-C₆ cycloalkyl, —CH₂(C₃-C₆ cycloalkyl), linear or branched —OC₁-C₃ alkyl, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl and cycloalkyl residues contained in the definitions of R¹¹-R¹⁴ are unsubstituted or substituted with one or more substituents independently selected from —F, —Cl, —Br, —I, —CH₃, —CF₃, —OH and —OCH₃, —OCF₃, —NH₂, —NHCH₃, —N(CH₃)₂; wherein all alkyl, alkenyl, alkynyl and cycloalkyl residues contained in the definitions of R¹¹-R¹⁴ can contain one or more heteroatoms independently selected from O, S and N in replacement of a carbon atom, and wherein such replacement cannot result in one of the groups selected from C═O and S═O directly bound to an aromatic ring;

wherein R¹¹-R¹⁴ are preferably selected from —H, —F, —Cl, —Br, —CH₃, —CF₃, —OH, —OCH₃, —OCF₃, cyclopropyl, oxiranyl, —C(CH₃)₃, —N(CH₃)₂, —NH₂, —CN, —CH₂OCH₃, —OCH(CH₃)₂, —CH₂NH₂, —CH₂N(CH₃)₂, —CH₂OH, —NO₂, —CH₂—N-morpholinyl;

R⁶ and R⁷ are independently selected from —H, —F, —CH₃; or R⁶ and R⁷ form together a cyclic residue including the carbon atom to which they are bound and wherein the cyclic residue is C₃ cycloalkyl;

R⁸ is selected from —H, C₁-C₃ alkyl preferably —CH₃, C₂-C₃ alkenyl, C₂-C₃ alkynyl, —F, —CF₃ and aromatic and heteroaromatic residues preferably six-membered aromatic cycles and five to six membered heteroaromatic cycles;

wherein said aromatic and heteroaromatic residues contained in the definition of R⁸ can optionally be linked through a C₁ alkylene or a C₂ alkylene linker to the carbon atom to which R⁸ is bound;

wherein all aromatic and heteroaromatic residues contained in the definition of R⁸ are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all heteroaromatic residues contained in the definition of R⁸ can contain one or more heteroatoms independently selected from O, S and N in replacement of a carbon atom;

wherein all alkyl, alkenyl, alkynyl residues contained in the definition of R⁸ are linear or branched, and are unsubstituted or substituted with one or more substituents independently selected from: —F, —Cl, —Br, —I, —CN, —NCO, —NCS, —OH and —NH₂;

wherein R⁸ is preferably —H, —F, —CH₃, —CH₂CH₃—CF₃, —C₆H₅;

wherein all alkyl, alkenyl, alkynyl, cycloalkyl, cycloalkenyl, bicycloalkyl, bicycloalkenyl, tricycloalkyl, aromatic and heteroaromatic residues contained in the definitions of R²-R⁸ and R¹¹-R¹⁴ can be partially or fully halogenated, particularly fluorinated, more particularly perfluorinated;

Z¹ and Z² are selected from the following groups:

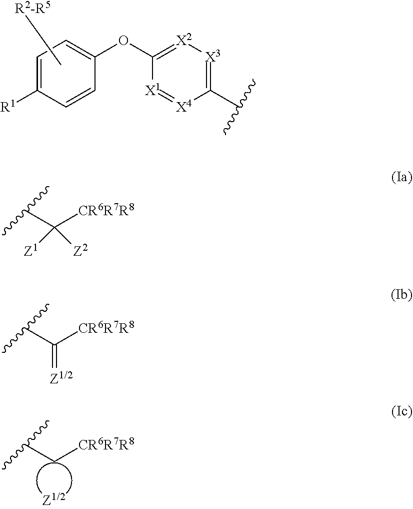

wherein Z¹ is selected from —H, linear or branched C₁-C₃ alkyl preferably —CH₃, cyclopropyl, oxiranyl, N-methyl-aziridinyl, thiiranyl, —N₃, —CF₃, —CF₂CF₃, and wherein Z² is independently selected from linear or branched C₁-C₃ alkyl preferably —CH₃, —CF₃, —CF₂CF₃, —OS(O)₂CH₃, —OS(O)₂CF₃, —OS(O)₂C₆H₄CH₃, —CN and —OR¹⁵ (general formula Ia), wherein R¹⁵ is selected from —H, C₁-C₈ preferably C₁-C₄ alkyl, C₂-C₈ preferably C₂-C₄ alkenyl, C₂-C₈ preferably C₂-C₄ alkynyl, C₃-C₆ cycloalkyl, C₅-C₆ cycloalkenyl, C₅-C₁₂ bicycloalkyl, C₇-C₁₂ bicycloalkenyl, C₈-C₁₄ tricycloalkyl, and aromatic and heteroaromatic residues preferably five- to six-membered aromatic cycles and five to six membered heteroaromatic cycles;

and wherein bicyclic and tricyclic residues include fused, bridged and spiro systems;

wherein said cycloalkyl, cycloalkenyl bicycloalkyl, bicycloalkenyl, tricycloalkyl, aromatic and heteroaromatic residues contained in the definition of R¹⁵ can optionally be linked through a C₁ alkylene or a C₂ alkylene or a C₃ alkylene linker to the O to which R¹⁵ is bound;

wherein all aromatic and heteroaromatic residues contained in the definition of R¹⁵ are unsubstituted or substituted with one or more substituents independently selected from: —F, —Cl, —Br, —I, —CN, —NCO, —NCS, —OH, —NH₂, —NO₂, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl, bicycloalkyl, bicycloalkenyl and tricycloalkyl residues, and alkylene linkers contained in the definition of R¹⁵ are linear or branched, and are unsubstituted or substituted with one or more substituents independently selected from: —F, —Cl, —Br, —I, —CN, —NCO, —NCS, —OH, —NH₂, ═O, linear or branched C₁-C₃ alkyl, C₂-C₃ alkenyl, C₂-C₃ alkynyl, cyclopropyl, linear or branched —OC₁-C₃ alkyl such as —OCH₃, —O(cyclopropyl), linear or branched —NH(C₁-C₃ alkyl), linear or branched —N(C₁-C₃ alkyl)(C₁-C₃ alkyl), —NH(cyclopropyl), —N(cyclopropyl)₂, linear or branched —N(C₁-C₃ alkyl)(cyclopropyl);

wherein all alkyl, alkenyl, alkynyl, cycloalkyl, cycloalkenyl, bicycloalkyl, bicycloalkenyl, tricycloalkyl and heteroaromatic residues, and alkylene linkers contained in the definition of R¹⁵ can contain one or more heteroatoms independently selected from O, S and N in replacement of a carbon atom;

wherein all alkyl, alkenyl, alkynyl, cycloalkyl, cycloalkenyl, bicycloalkyl, bicycloalkenyl, tricycloalkyl, and heteroaromatic residues, and alkylene linkers contained in the definition of R¹⁵ can be partially or fully halogenated, particularly fluorinated, more particularly perfluorinated wherein R¹⁵ is preferably —H, —CH₃, —CH₂CH₃, n-propyl, isopropyl, cyclopropyl, benzyl;

or wherein Z¹ and Z² are together ═O, ═S, ═NR¹⁶, or zwitterionic ═N^{[+]}R¹⁷O^{[-]} (general formula Ib); wherein R¹⁶ is selected from —H, —OH, —OCH₃, —CN, —S(O)CH₃, —S(O)CF₃, —S(O)C(CH₃)₃, —S(O)₂CH₃, —S(O)₂CF₃, linear or branched C₁-C₃ alkyl preferably —CH₃, cyclopropyl, —CF₃, —CF₂CF₃, —CH₂CF₃, —C₆H₅ and —CH₂C₆H₅; wherein R¹⁷ is selected from linear or branched C₁-C₃ alkyl, preferably —CH₃, cyclopropyl, —C₆H₅ and —CH₂C₆H₅;

or wherein Z¹ and Z² form together a cyclic residue including the carbon atom to which they are bound (general formula Ic); wherein the cyclic residue is selected from three-membered rings, four-membered rings, five-membered rings and six-membered rings, wherein all rings optionally can contain one or more heteroatoms independently selected from O, S and N in replacement of a carbon atom; wherein all rings are unsubstituted or substituted with one or more substituents independently selected from: —F, —Cl, —Br, —I, —CN, —NCO, —NCS, —OH, —OCH₃, —NH₂, —NHCH₃, —N(CH₃)₂, ═O, —CH₃ and —CF₃;

wherein all alkyl and cyclic residues contained in the definitions of Z¹ and Z² can be partially or fully halogenated, particularly fluorinated, more particularly perfluorinated.

1. The compound of claim 1 according to general formula (Ia) or a salt or solvate thereof.

2. The compound of claim 1 according to general formula (Ib) or a salt or solvate thereof.

3. The compound of claim 1 according to general formula (Ic) or a salt or solvate thereof.

4. The compound of claim 1 with the proviso that
(i) compounds as indicated in Table 1 are excluded,
(ii) compounds as indicated in Table 2 are excluded and/or
(iii) the compound as indicated in Table 3 are excluded.

5. The compound of claim 1
wherein R¹ is selected from methyl, ethyl, n-propyl, n-butyl, n-pentyl, n-hexyl, iso-propyl, sec-butyl, tert-butyl, tert-pentyl, tert-octyl, 3-pentyl, —CF₃, —CF₂CF₃, —(CF₂)₂CF₃, —CH(CF₃)₂, —CH₂SCH₃, —CH₂CH₂SCH₃, —CH₂SCH₂CH₃, —CH₂CH₂SCH₂CH₃, methoxymethyl, methoxyethyl, methoxypropyl, ethoxymethyl, ethoxyethyl, propoxymethyl, dimethyl-aminomethyl, dimethyl-aminoethyl, diethyl-aminomethyl, ethyl-methyl-aminomethyl, cyclopropyl, methyl-cyclopropyl, ethyl-cyclopropyl, trifluoromethyl-cyclopropyl, perfluoroethyl-cyclopropyl, cyclobutyl, cyclopentyl, cyclohexyl, cycloheptyl, bicyclopentyl, bicyclohexyl, bicycloheptyl preferably norbornyl, bicyclooctyl, bicyclooctenyl, bicyclononyl, methylbicyclononyl, adamantyl, tricyclodecyl, oxiranyl, oxetanyl, tetrahydrofuranyl, methyltetrahydrofuranyl, trimethyltetrahydrofuranyl, tetrahydropyranyl, aziridinyl, N-methylaziridinyl, azetidinyl, N-methylazetidinyl, difluoroazetidinyl, pyrrolidinyl, N-methylpyrrolidinyl, piperidinyl, N-methylpiperidinyl, difluoropiperidinyl, thiiranyl, thietanyl, tetrahydrothiophenyl, tetrahydrothiopyranyl, dioxanyl, piperazinyl, dimethylpiperazinyl, dithianly, morpholinyl, N-methylmorpholinyl, thiomorpholinyl, N-methylthiomorpholinyl, oxa-azaspiroheptyl, N-methyloxa-azaspiroheptyl, azaspiroheptyl, N-methylazaspiroheptyl, thia-azaspiroheptyl, N-methylthia-azaspiroheptyl, difluorothia-azaspiroheptyl, azaspirooctyl, N-methylazaspirooctyl, oxa-azaspirooctyl, N-methyloxa-azaspirooctyl, oxa-azaspirononyl, N-methyloxa-azaspirononyl, azaspirononyl, N-methylazaspirononyl, oxa-azaspirodecyl, N-methyloxa-azaspirodecyl, azaspirodecyl, N-methylazaspirodecyl, dihydro-oxazinyl, N-methyldihydro-oxazinyl, oxazolidinyl, N-methyloxazolidinyl, dioxolanyl, imidazolidinyl, N-methylimidazolidinyl, N,N-dimethylimidazolidinyl, azepanyl, N-methylazepanyl, azaspirohexyl, N-methylazaspirohexyl, oxa-azadispirodecyl, N-methyloxa-azadispirodecyl, azadispirodecyl, N-methylazadispirodecyl, oxa-azabicyclooctyl, N-methyloxa-azabicyclooctyl, azabicyclooctyl, N-methylazabicyclooctyl, azabicycloheptyl, N-methylazabicycloheptyl, azabicyclononyl, N-methylazabicyclononyl, azaadamantyl, —O(adamantyl), oxa-azabicyclononyl, N-methyloxa-azabicyclononyl, oxa-azabicycloheptyl, N-methyloxa-azabicycloheptyl, diazabicyclooctyl, N-methyldiazabicyclooctyl, N,N-dimethyldiazabicyclooctyl, diazabicycloheptyl, N-methyldiazabicycloheptyl, N,N-dimethyldiazabicycloheptyl; 4-oxocyclohexyl; 3-oxocyclopentyl; 2-oxocyclobutyl, 4-oxobicyclo[4.1.0]heptan-1-yl.

7. The compound of claim 1,
wherein R¹ is selected from the group consisting of C₄-C₁₂ alkyl, C₄-C₁₂ alkenyl, C₄-C₁₂ alkynyl, cyclic, bicyclic and tricyclic residues, wherein the alkyl, alkenyl and alkynyl residues are preferably branched, including:

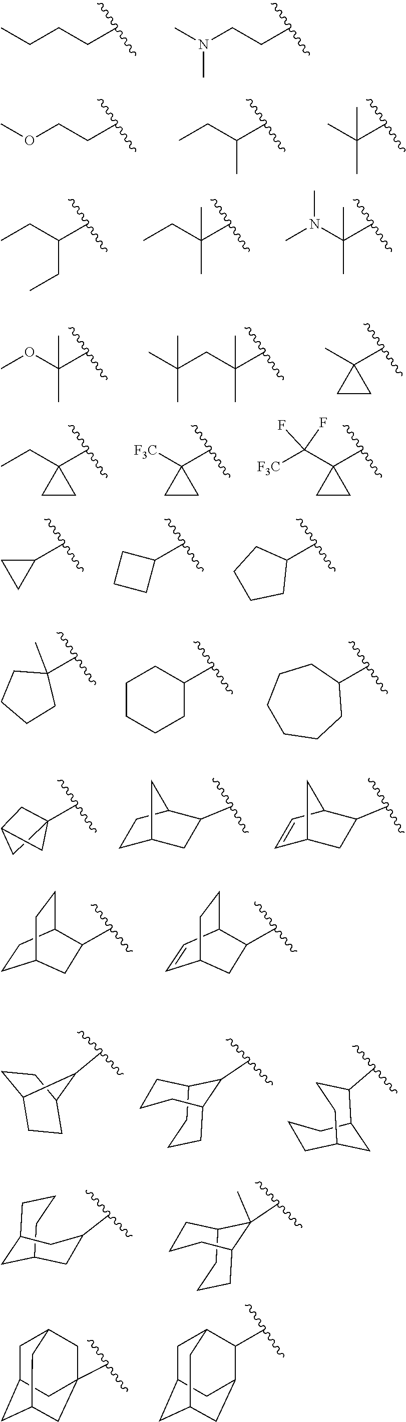

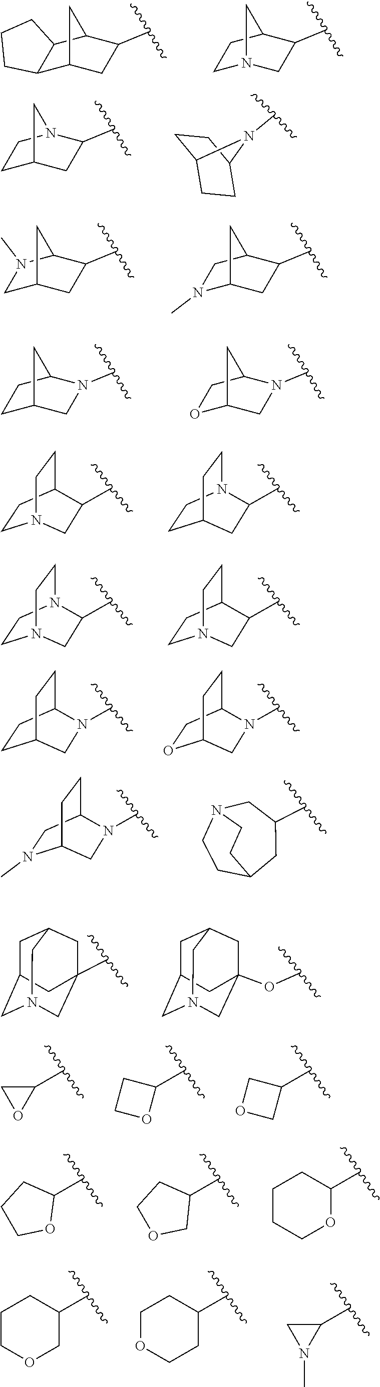

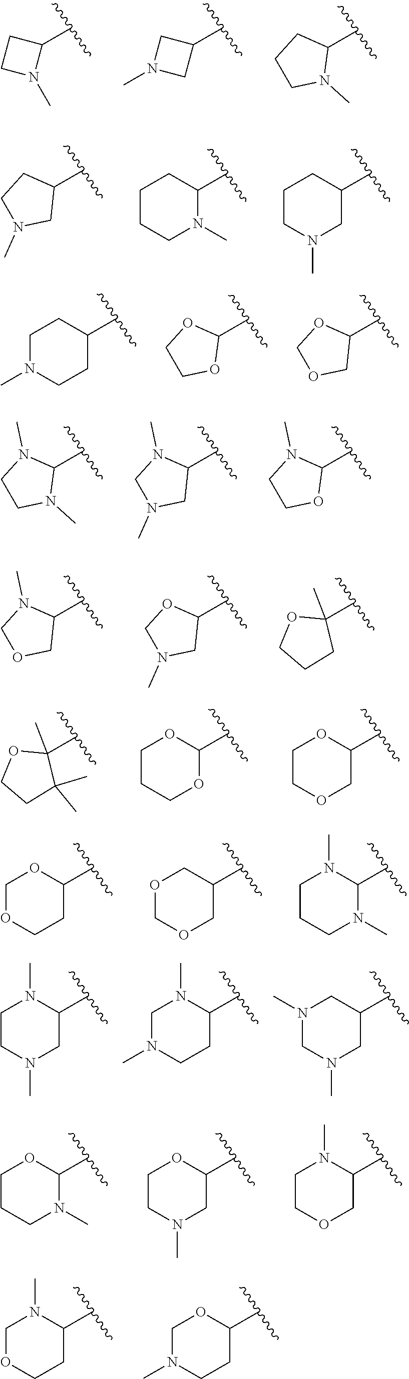

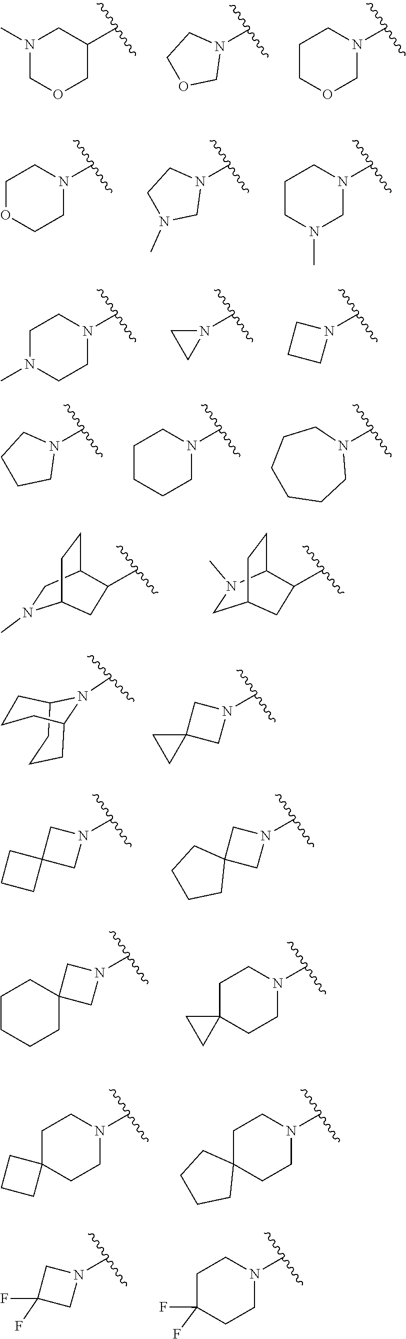

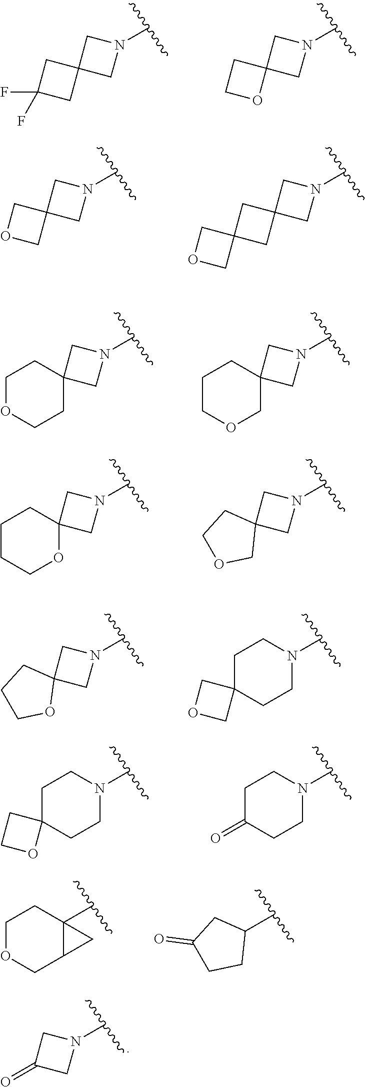

8. The compound of claim 1
wherein R²-R³ each are —H, R⁴ is preferably —H or —F, and/or R⁵ is —H, —F, —Cl, —Br, —CH₃, —CF₃, —CH═CH₂, —C—CH, —CH₂OH, —CH₂NHCH₃, —OH, —OCH₃, —OCF₃, cyclopropyl, oxiranyl, —CH₂—N— morpholinyl, —C(CH₃)₃, —CH₂OCH₃, —NO₂, —CN, —NH₂, —N(CH₃)₂, —OCH(CH₃)₂, —CH₂NH₂, —CH₂N(CH₃)₂.

9. The compound of claim 1
wherein the six-membered aromatic ring to which substituents R¹ to R⁵ are bound as defined in general formula (I) is selected from the group consisting of

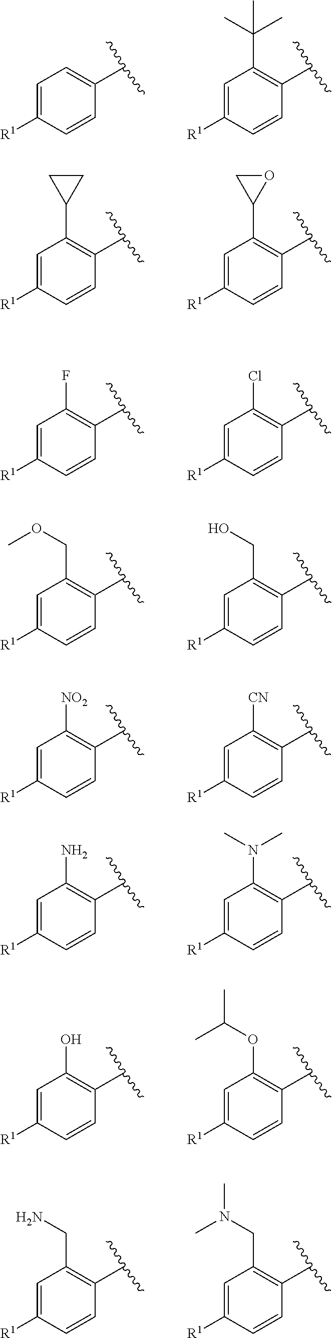

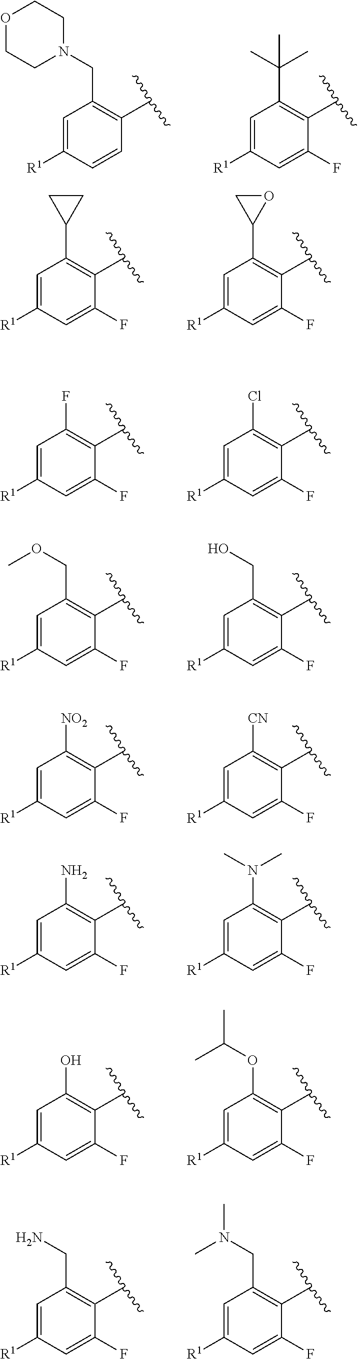

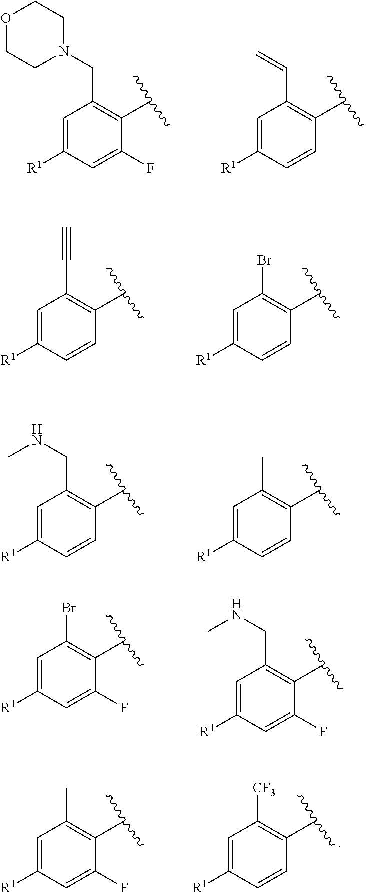

10. The compound of claim 1
wherein the six-membered aromatic ring containing X¹-X⁴ as defined in general formula (I) is selected from the group consisting of:

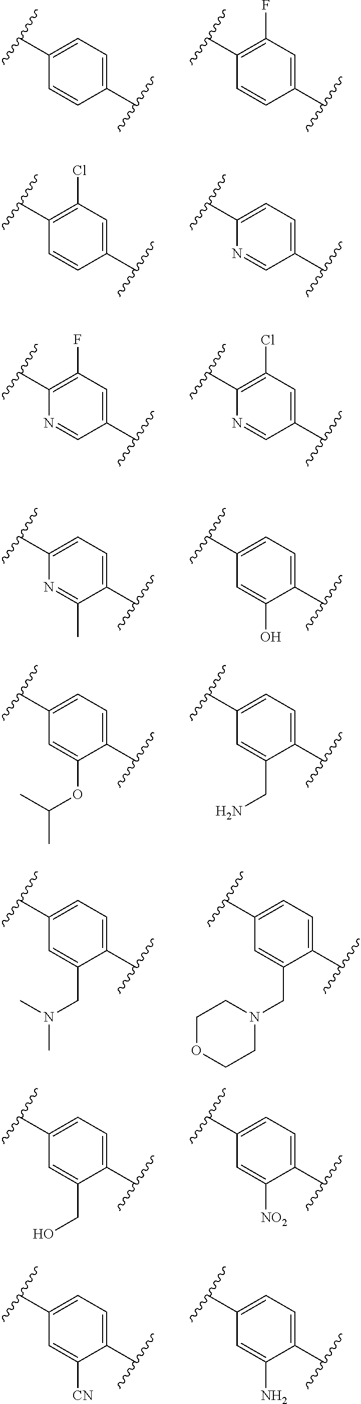

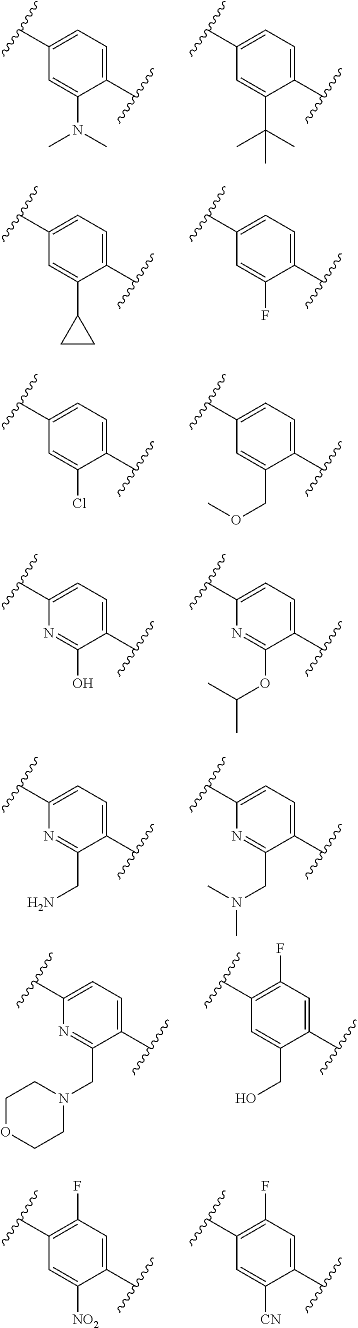

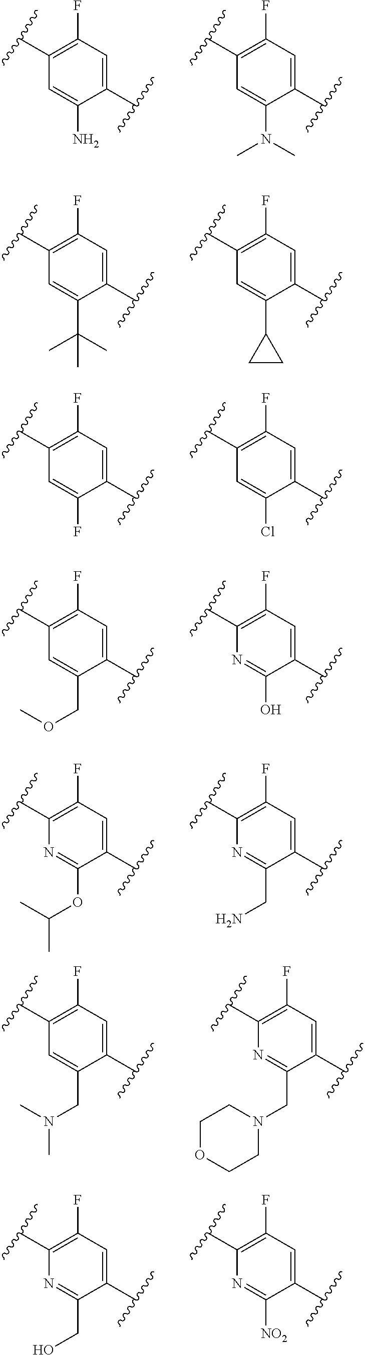

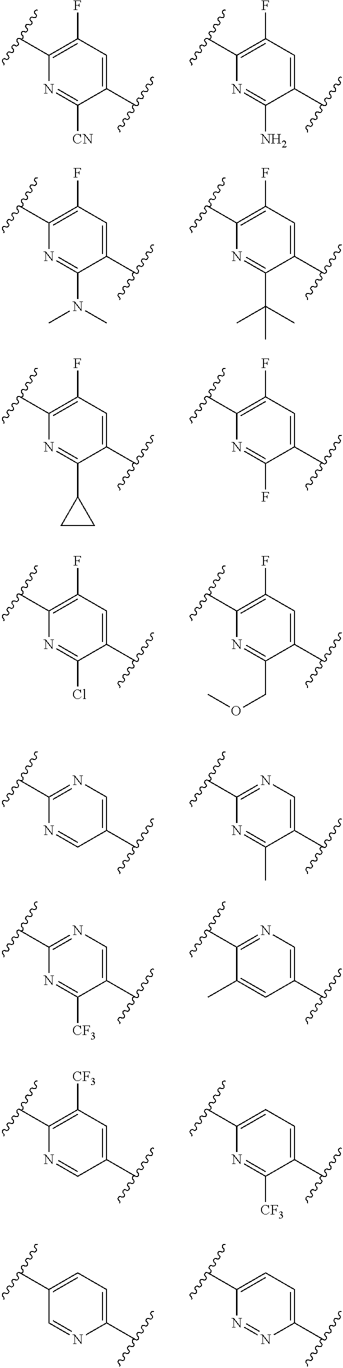

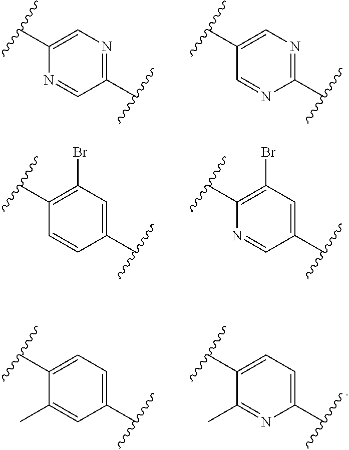

11. The compound of claim 1
wherein Z¹ is —H, —CH₃, —CF₃ or cyclopropyl; and/or wherein Z² is —OH, —OS(O)₂CH₃ and —CN; e.g.:

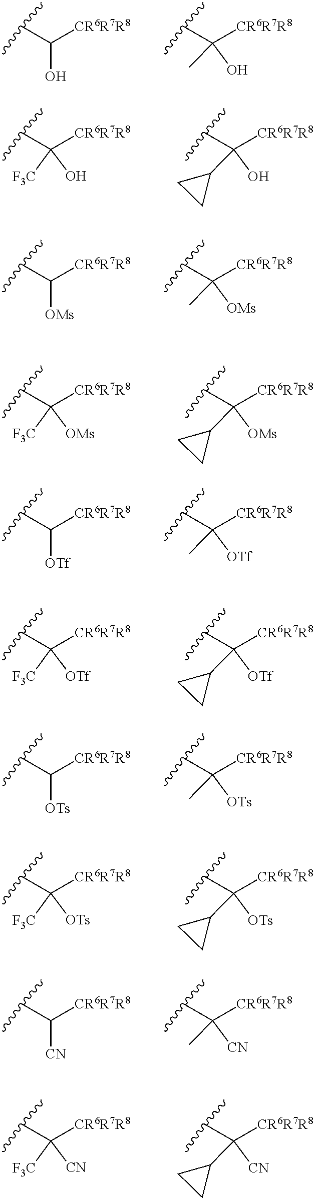

12. The compound of claim 1
wherein Z¹ and Z² are together ═O, ═NR¹⁶ or zwitterionic ═N^{[+]}R¹⁷O^{[-]}; wherein R¹⁶ is preferably selected from —H, —OH, —OCH₃, —CH₃, cyclopropyl, and —CH₂C₆H₅; wherein R¹⁷ is preferably —CH₃, —C(CH₃)₃ and —CH₂C₆H₅:

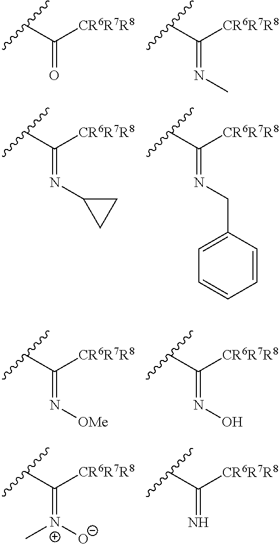

13. The compound of claim 1
wherein Z¹ and Z² form together a three membered or four membered or five membered cyclic residue including the carbon atom to which they are bound; wherein this cyclic residue is preferably selected from cyclopropyl, cyclobutyl, oxiranyl, oxetanyl, aziridinyl, azetidinyl, thietanyl, thiazolidinyl, methylthiazolidinyl, thiazolidine-dionyl, methylthiazolidine-dionyl and oxazolidinyl, methyloxazolidinyl, oxazolidine-dionyl and methyloxazolidine-dionyl; and
wherein this cyclic residue is optionally substituted preferably with —F, —OH, —OCH₃, —NH₂, —NHCH₃, —N(CH₃)₂, ═O, —CH₃ and —CF₃;
and wherein this cyclic residue is even more preferably selected from:

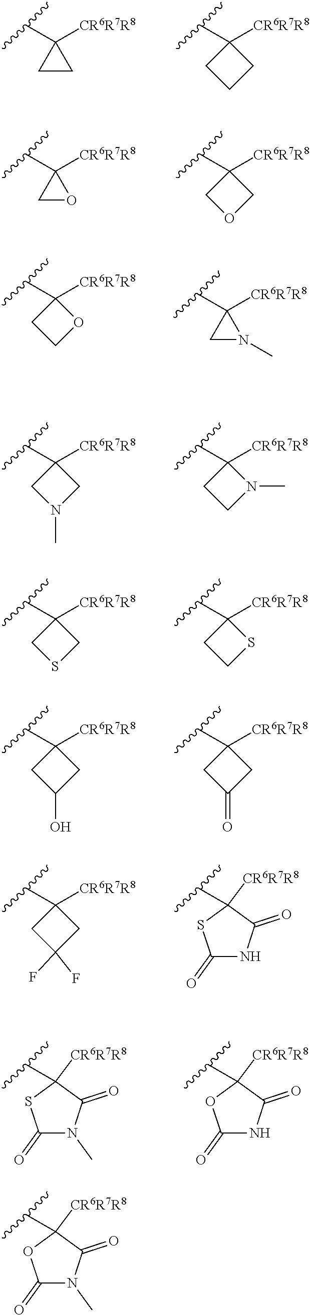

14. The compound of claim 1
wherein R⁶, R⁷ and R⁸ are each —F.

15. The compound of claim 1
wherein R⁶ and R⁷ form together a cyclic residue including the carbon atom to which they are bound and wherein the cyclic residue is cyclopropyl.

16. The compound of claim 1
wherein R¹ contains no heteroatom.

17. The compound of claim 16
wherein R¹ is selected from cyclic, bicyclic and tricyclic structures.

18. The compound of claim 16
wherein R¹ is selected from the group consisting of cyclohexyl, norbornyl, bicyclooctyl, bicyclononyl, methylbicyclononyl, tricyclodecyl and adamantyl.

19. The compound of claim 18
wherein R¹ is adamantyl.

20. The compound of claim 1
wherein R¹ is selected from residues, which contain four or more, preferably six or more and even more preferably seven or more carbon atoms.

21. The compound of claim 1 wherein R¹ contains one or more, preferably one to two heteroatoms independently selected from O, S and N in replacement of a carbon atom contained in R₁.

22. The compound of claim 21
wherein R¹ is selected from cyclic, bicyclic and tricyclic structures, or wherein R¹ is selected from residues containing cyclic, bicyclic and tricyclic structures.

23. The compound of claim 21
wherein R¹ is selected from tetrahydropyranyl, N-methylpiperidinyl, morpholinyl, 4-oxocyclohexyl, azabicycloheptyl, N-methylazabicycloheptyl, oxa-azabicycloheptyl, N-methyldiazabicycloheptyl, azabicyclooctyl, diazabicyclooctyl, N-methyldiazabicyclooctyl, oxa-azabicyclooctyl, azabicyclononyl, aza-adamantyl and —O(adamantyl).

24. The compound of claim 23
wherein R¹ is aza-adamantyl and —O(adamantyl).

25. The compound of claim 1 which has the structure I-1: 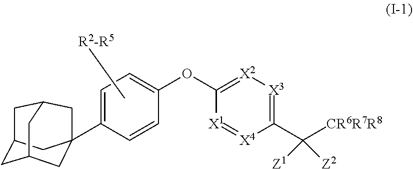
wherein Z¹ and Z² are defined as in general formula (I), including general formula (Ia), general formula (Ib) and general formula (Ic), including the substitutions and preferred definitions,
and wherein R¹⁵ is defined as in general formula (Ia) including the substitutions and preferred definitions, and wherein R¹⁶ and R¹⁷ are defined as in general formula (Ib) including the substitutions and preferred definitions,
and wherein R²-R⁸, R¹¹-R¹⁴ and X¹-X⁴ are defined as in general formula (I) including the substitutions and preferred definitions.

26. The compound of claim 1 which has the structure I-2: 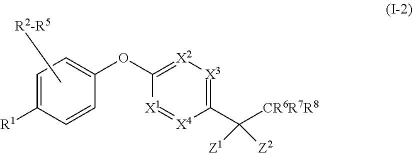
wherein R¹ is defined as in general formula (I) including the substitutions and preferred definitions, wherein R¹ is selected from cyclic, bicyclic and tricyclic structures, and wherein R¹ contains six or more carbon atoms, which are optionally independently replaced by a heteroatom selected from O, S and N as defined in general formula (I),
wherein R⁶ is defined as in general formula (I) including the substitutions and preferred definitions, wherein R⁶ is different from —H, optionally with the additional proviso that R⁶ is different from —CH₃,
and wherein Z¹ and Z² are defined as in general formula (I), including general formula (Ia), general formula (Ib) and general formula (Ic), including the substitutions and preferred definitions,
and wherein R¹⁵ is defined as in general formula (Ia) including the substitutions and preferred definitions, and wherein R¹⁶ and R¹⁷ are defined as in general formula (Ib) including the substitutions and preferred definitions,
and wherein R²-R⁵, R⁷-R¹⁴ and X¹-X⁴ are defined as in general formula (I) including the substitutions and preferred definitions.

27. The compound of claim 1
which has the structure I-3: 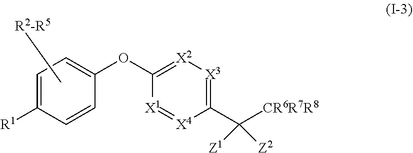
wherein R¹ is selected from cyclic, bicyclic and tricyclic structures, and wherein R¹ contains six or more carbon atoms, which are optionally independently replaced by a heteroatom selected from O, S and N as defined in general formula (I),
wherein R⁸ is defined as in general formula (I) including the substitutions and preferred definitions, wherein R⁸ is different from —H, optionally with the additional proviso that R⁸ is different from —CH₃,
and wherein Z¹ and Z² are defined as in general formula (I), including general formula (Ia), general formula (Ib) and general formula (Ic), including the substitutions and preferred definitions,
and wherein R¹⁵ is defined as in general formula (Ia) including the substitutions and preferred definitions, and wherein R¹⁶ and R¹⁷ are defined as in general formula (Ib) including the substitutions and preferred definitions.

28. A compound as shown in any one of Table 4 to Table 28 or a salt or solvate thereof.

29. A pharmaceutical composition comprising the compound of claim 1 in combination with a pharmaceutical carrier suitable for human medicine or veterinary medicine.

30. (canceled)

31. A method for treating diseases and malignant, non-malignant and hyperproliferative disorders of the skin, mucosa, skin and mucosal appendages, cornea, and epithelial tissues, including cancer such as non-melanoma skin cancer including squamous and basal cell carcinoma and precancerous lesions including actinic keratosis, skin and/or mucosal disorders with cornification defects and/or abnormal keratinocyte proliferation, skin and/or mucosal diseases associated with, accompanied by and/or caused by viral infections, atopic dermatitis and acne and in the promotion of wound healing of the skin and mucosa, comprising administering a compound according to claim 1 to a patient in need of such treatment.

32. A method for treating hyperproliferative disorders, cancers or precancerous lesions of the skin, oral mucosa, tongue, lung, stomach, breast, cancer of the neuroendocrine system, such as medullary thyroid cancer, brain, pancreas, liver, thyroid, and genitourinary tract including cancer of the cervix and ovaries, comprising administering the compound according to claim 1 to a patient in need of such treatment.

33. A method for treating malignant and non-malignant muscular diseases including muscular dystrophies, or in muscle regeneration, or in hyperproliferative disorders of the muscle, such as muscle hyperplasia and muscle hypertrophy, comprising administering the compound according to claim 1 to a patient in need of such treatment.

34. A method for treating immune system-related disorders, including disorders of the haematopoietic system including the haematologic system, such as cancer of the haematopoietic and haematologic system such as leukemias and lymphomas, such as malignancies of the myeloid lineage e.g. acute and chronic myeloid leukemia and acute and chronic promyelocytic leukemia, and malignancies of the lymphoid lineage, e.g. acute and chronic T-cell leukemia and acute and chronic B-cell leukemia, and cutaneous T-cell lymphoma, comprising administering a compound according to claim 1 to a patient in need of such treatment.

35. A method for improving therapeutic immune system-related applications including immunotherapy and other immunotherapy methods, comprising administering an immunologic adjuvant or vaccine adjuvant comprising a compound according to claim 1.

36. A method of treating a hyperproliferative disorder comprising administering a subject in need thereof, particularly a human subject, a therapeutically effective amount of a compound according to claim 1.
